(19) United States
(12) Patent Application Publication
(10) Pub. No.: US 2013/0303876 A1
(43) Pub. Date: Nov. 14, 2013

(54) CAROTID BODY MODULATION PLANNING AND ASSESSMENT

(71) Applicants: Mark GELFAND, New York, NY (US); Howard LEVIN, Teaneck, NJ (US); Zoar Jacob ENGELMAN, Salt Lake City, UT (US); Marat FUDIM, Duesseldorf (DE)

(72) Inventors: Mark GELFAND, New York, NY (US); Howard LEVIN, Teaneck, NJ (US); Zoar Jacob ENGELMAN, Salt Lake City, UT (US); Marat FUDIM, Duesseldorf (DE)

(21) Appl. No.: 13/852,895

(22) Filed: Mar. 28, 2013

Related U.S. Application Data
(60) Provisional application No. 61/616,897, filed on Mar. 28, 2012.
(60) Provisional application No. 61/667,996, filed on Jul. 4, 2012.
(60) Provisional application No. 61/791,420, filed on Mar. 15, 2013.

(51) Int. Cl. A61B 18/00; A61B 5/02

(52) U.S. Cl. CPC A61B 18/00; A61B 5/02
USPC 600/407

(57) ABSTRACT

Planning for and/or assessment of an ablation procedure on one or both carotid bodies or carotid body chemoreceptors or carotid body nerves to treat patients having a sympathetically mediated cardiac, metabolic, and pulmonary disease (e.g. hypertension, CHF, diabetes, sleep disordered breathing) resulting from peripheral chemoreceptor hypersensitivity, carotid body hyperactivity, high carotid body afferent nerve signaling or heightened sympathetic activation.

### CROSS REFERENCE TO RELATED APPLICATIONS

This application claims the priority of U.S. Provisional Application No. 61/616,897, filed Mar. 28, 2012; U.S. Provisional Application No. 61/667,996, filed Jul. 4, 2012; and U.S. Provisional Application No. 61/791,420, filed Mar. 15, 2013, the disclosures of which are incorporated herein by reference in their entireties.

This application is related to U.S. Pub. No. 2012/0172680, published Jul. 5, 2012, the disclosure of which is incorporated by reference herein in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

It is known that an imbalance of the autonomic nervous system is associated with several disease states. Restoration of autonomic balance has been a target of several medical treatments including modalities such as pharmacological, device-based, and electrical stimulation.

Sympathetically mediated diseases such as hypertension, heart failure, type II diabetes, chronic kidney disease and others represent significant and growing global health issues. The rates of control of blood pressure and the therapeutic efforts to prevent progression of heart failure, chronic kidney disease, diabetes and their sequelae remain unsatisfactory. Recent introduction of medical procedures, such as renal denervation (Gelfand and Levin U.S. Pat. No. 7,162,303), and devices such as deep brain stimulation, baroreceptor stimulation (Kieval, Burns and Serdar U.S. Pat. No. 8,060,206), implantable neurostimulation of carotid afferent nerves (Hlavka and Elliott US 2010/0070004), direct vagal stimulation and concepts such as restoring autonomic balance by increasing parasympathetic activity to treat disease associated with parasympathetic attrition (Yun and Yuarn-Bor U.S. Pat. No. 7,149,574; U.S. Pat. No. 7,363,076; U.S. Pat. No. 7,738,952) begin to address these gaps in selective patients. So far these interventions have not resulted in significant reduction of numbers of patients depending on multiple drugs to control their blood pressure.

Proper planning for and/or assessment of these and other interventions will allow for safe and effective therapy.

### SUMMARY OF THE DISCLOSURE

One aspect of the disclosure is a method of planning for a procedure that reduces afferent nerve activity of a carotid body, comprising providing at least one image of a subject's carotid vasculature region; determining a characteristic of at least one of a vagus nerve, hypoglossal nerve, and sympathetic nerve; and making a determination about the afferent nerve activity reducing procedure based on the characteristic of at least one of the vagus nerve, hypoglossal nerve, and sympathetic nerve.

In some embodiments determining a characteristic of at least one of a vagus nerve, hypoglossal nerve, and sympathetic nerve comprises determining a position of a carotid body relative to at least one of a vagus nerve, hypoglossal nerve, and sympathetic nerve, and making a determination about the afferent nerve activity reducing procedure comprises making a determination about the afferent nerve activity reducing procedure based on the position of the carotid body relative to at least one of the vagus nerve, hypoglossal nerve, and sympathetic nerve.

In some embodiments determining a characteristic of at least one of a vagus nerve, hypoglossal nerve, and sympathetic nerve comprises delivering a stimulator in the area of the carotid vasculature and monitoring for a response indicative of stimulation of at least one of the vagus nerve, hypoglossal nerve, and sympathetic nerve. Delivering a stimulator can comprise delivering at least one of thermal and electrical energy in the area of the carotid vasculature, and it can comprise delivering a chemical agent in the area of the carotid vasculature. Monitoring for a response indicative of stimulation of at least one of the vagus nerve, hypoglossal nerve, and sympathetic nerve can comprise monitoring for a motor response, or monitoring for at least one of a cardiovascular response and a respiratory response.

One aspect of the disclosure is a method of planning for a procedure that reduces afferent nerve activity of a carotid body, comprising providing at least one image of a subject's carotid vasculature; determining if an atherosclerosis is present in the carotid vasculature; and making a determination about the afferent nerve activity reducing procedure based on the presence or absence of an atherosclerosis in the at least one image.

In some embodiments the investigating step investigates an image that shows a common carotid artery, and the determining step determines if an atherosclerosis is present in the common carotid artery.

In some embodiments the investigating step investigates an image that shows at least a portion of an aortic arch, and the determining step determines if an atherosclerosis is present in the aortic arch.

In some embodiments the investigating step investigates an image that shows an external carotid artery, and the determining step determines if an atherosclerosis is present in the external carotid artery.

In some embodiments making a determination about the afferent nerve activity reducing procedure comprises making a determination about whether or not to perform the procedure.

In some embodiments making a determination about the afferent nerve activity reducing procedure comprises making a determination about a particular vascular approach to a treatment site for the procedure.

One aspect of the disclosure is a method of planning for a procedure that reduces afferent nerve activity of a carotid body, comprising providing at least one image of a subject's carotid vasculature region; assessing a distance from a carotid body to at least one anatomical structure using the at least one image; and making a determination about the afferent nerve activity reducing procedure based on the assessed distance from the carotid body to the at least one anatomical structure.

In some embodiments assessing a distance from a carotid body to at least one anatomical structure using the at least one image comprises assessing a distance from the carotid body to an internal carotid artery.

In some embodiments assessing a distance from a carotid body to at least one anatomical structure using the at least one image comprises assessing a distance from the carotid body to an external carotid artery.

In some embodiments assessing a distance from a carotid body to at least one anatomical structure using the at least one image comprises assessing a distance from the carotid body to a carotid artery bifurcation.

In some embodiments making a determination about the afferent nerve activity reducing procedure based on the assessed distance from the carotid body to the at least one anatomical structure comprises deciding where to position a treatment device within the vasculature to perform the procedure.

In some embodiments the assessing step is performed automatically by an algorithm.

In some embodiments the assessing step is performed manually.

One aspect of the disclosure is a method of planning for a procedure that reduces afferent nerve activity of a carotid body, comprising determining that an ablation procedure on one of a right carotid body and a left carotid body will better treat a disease associated with heightened carotid body activation than an ablation procedure on the other of the right carotid body and the left carotid body; and performing the procedure on the right or the left carotid body based on the determining step.

In some embodiments the method further comprises assessing whether a disease associated with heightened carotid body activation has been satisfactorily treated.

In some embodiments the method further comprises performing the procedure on the other of the left and right carotid body if it is determined that the disease has not been satisfactorily treated.

In some embodiments determining that an ablation procedure on one of a right carotid body and a left carotid body will better treat a disease associated with heightened carotid body activation comprises testing chemosensitivity of at least one of a left carotid body and a right carotid body.

In some embodiments determining that an ablation procedure on one of a right carotid body and a left carotid body will better treat a disease associated with heightened carotid body activation comprises assessing the size of the right carotid body.

In some embodiments determining that an ablation procedure on one of a right carotid body and a left carotid body will better treat a disease associated with heightened carotid body activation comprises selectively stimulating at least one of the left and right carotid bodies, and measuring a response to the selective stimulation of the at least one left and right carotid bodies. Selectively stimulating can comprise exposing the left or right carotid body to a stimulant.

One aspect of the disclosure is a method of planning for a procedure that reduces afferent nerve activity of a carotid body, comprising providing at least one image of a subject's carotid vasculature region; determining if a carotid body is located at least partially within a carotid septum using the at least one image; and making a determination about the afferent nerve activity reducing procedure based on the determination if the carotid body is located at least partially within the carotid septum.

In some embodiments making a determination about the afferent nerve activity reducing procedure comprises making a determination about whether to perform the procedure or not based on whether the carotid body is located at least partially within a carotid septum or not.

In some embodiments making a determination step comprises making a determination not to perform the procedure if the carotid body is not substantially located within the carotid septum.

In some embodiments making a determination about the afferent nerve activity reducing procedure comprises making a determination to at least partially ablate the carotid septum.

One aspect of the disclosure a method of planning for a procedure that reduces afferent nerve activity of a carotid body, comprising providing at least one image of a subject's carotid vasculature; and making a determination about an aspect of energy delivery for the afferent nerve activity reducing procedure based on the image of the subject's carotid vasculature.

In some embodiments making a determination about an aspect of energy delivery comprises selecting one of a plurality of different energy modalities for the procedure based on the at least one image.

In some embodiments making a determination comprises selecting RF energy as the energy modality for the procedure.

In some embodiments making a determination about an aspect of energy delivery comprises selecting one of a plurality of different energy parameters for the procedure, such as power or duration, based on the at least one image.

One aspect of the disclosure is a method of planning for a procedure that reduces afferent nerve activity of a carotid body, comprising providing at least one image of a subject's carotid vasculature; and making a determination about a vascular approach for a treatment device for the afferent nerve activity reducing procedure based on the at least one image of the subject's carotid vasculature.

In some embodiments making a determination about a vascular approach comprises determining an access point for a treatment device for the afferent nerve activity reducing procedure.

In some embodiments making a determination about a vascular approach comprises determining a navigation route to a treatment site for the treatment device.

In some embodiments making a determination about a vascular approach for a treatment device for the afferent nerve activity reducing procedure based on the image of the subject's carotid vasculature comprises recognizing the presence or absence of an atherosclerosis in the subject's vasculature.

In some embodiments making a determination about a vascular approach for a treatment device comprises selecting one or a plurality of treatment devices based on the at least one image of the subject's carotid vasculature.

In some embodiments making a determination about a vascular approach for a treatment device comprises determining whether or not to access an external carotid artery.

In some embodiments making a determination about a vascular approach for a treatment device is performed by an algorithm.

In some embodiments making a determination about a vascular approach for a treatment device comprises manually making a determination about a vascular approach for a treatment device.

One aspect of the disclosure is a method of performing a procedure on a subject that reduces afferent nerve activity of a carotid body, comprising providing an image of the subject's carotid vasculature; measuring a distance of about 15 mm from a carotid artery bifurcation along a lumen of an external carotid artery to estimate a first position range; positioning an energy delivery device in an external carotid artery in the first position range; and activating the energy delivery device to ablate tissue within the carotid septum. Activating the energy delivery device to ablate tissue within the carotid septum can comprise ablating at least a part of a carotid body.

### BRIEF DESCRIPTION OF THE DRAWINGS

FIG. 1 is a cutaway illustration of vasculature and neural structures of a right side of a patient's neck.

FIGS. 2 to 4 are illustrations of surgical access to a patient's left carotid body.

FIG. 5 is an illustration of a patient's right carotid arteries and bifurcation with a schematic view of an endovascular catheter inserted into the vasculature to ablate a carotid body.

FIG. 6 is a schematic view of an endovascular ablation device ablating a carotid body along with a thermal protection catheter placed proximate a carotid sinus.

FIG. 7 is a schematic view of an endovascular radiofrequency catheter ablating a carotid body.

FIG. 8 is a schematic view of an endovascular bi-polar radiofrequency catheter ablating a carotid body.

FIG. 9 is a schematic view of an endovascular cooled-radiofrequency catheter ablating a carotid body.

FIG. 10 is a schematic view of an endovascular catheter used for transvascular ablation of a carotid body.

FIG. 11 is a schematic view of an endovascular catheter used for cryogenic ablation of a carotid body.

FIG. 12 is a schematic view of an occlusion device used to embolize blood supply to a carotid body.

FIG. 13 is a schematic view of a segregation catheter used to deliver an agent that may be used to visualize, chemically ablate, or embolize a carotid body.

FIG. 14 is a schematic view of a percutaneous ablation device ablating a carotid body.

FIG. 15 is a schematic view of a percutaneous ablation device ablating a carotid body along with a fiduciary endovascular catheter.

FIG. 16 is a schematic view of a percutaneous ablation device ablating a carotid body along with a fiduciary endovascular catheter.

FIG. 17 is a schematic view of a percutaneous ablation device ablating a carotid body aided with ultrasound imaging.

FIG. 18 is a schematic view of a percutaneous ablation device ablating a carotid body aided with ultrasound imaging.

FIG. 19 is a schematic view of a High Intensity Focused Ultrasound (HIFU) device ablating a carotid body.

FIG. 20 is a computer tomography image of a patient's carotid artery showing a carotid body.

FIG. 21 is a flow chart of a method for treating a patient involving assessing the patient's chemosensitivity as a selection criterion for a carotid body ablation procedure.

FIG. 22 is a flow chart of a method for treating a patient involving assessing the patient's chemosensitivity and response to a temporary carotid body block as a selection criterion for a carotid body ablation procedure.

FIG. 23 is a schematic illustration of a patient's carotid arteries showing a carotid body.

FIG. 24 is a schematic illustration of physiological connections between carotid chemoreceptors, the central nervous system, and various organs and effects.

FIGS. 25A, 25B, and 25C are graphs from a study on rats: Abdala N M, A. Gourine, J. Paton. Peripheral chemoreceptor inputs contribute to the development of high blood pressure in spontaneously hypertensive rats, Annual Meeting of Physiological Society July 12 in England. 2011.

FIG. 26 is a diagram illustrating a connection of carotid body hyperactivity with hypertension.

FIG. 27 is a schematic illustration showing carotid body hyperactivity implicated in a cycle of sympathetically mediated disease progression.

FIG. 28 is a schematic illustration showing a relationship between carotid body activity and ventilatory effects.

FIG. 29 is a schematic illustration showing a relationship between carotid body activity and insulin resistance.

FIG. 30 is a schematic illustration showing a relationship between carotid body activity and sodium and fluid retention in CHF.

FIG. 31 is a schematic illustration showing a relationship between carotid body activity and Chronic Renal Disease and End Stage Renal Disease.

FIG. 32 is a schematic illustration showing a relationship between carotid body activity and congestion in decompensated heart failure.

FIG. 33 is a schematic illustration showing a relationship between carotid body activity and baroreflex and its effect on organs.

FIGS. 34A and 34B are schematic illustrations depicting an intercarotid septum.

FIG. 35 is an illustration of an energy delivery device adapted for positioning on the bifurcation of carotid artery to ablate carotid septum.

FIGS. 36A and 36B are schematic views showing suitable placement of ablation elements on an intercarotid septum for safe and effective carotid body modulation.

FIGS. 37A and 37B are illustrations of a method of testing individual response of right and left carotid bodies by injecting a drug.

FIGS. 38A and 38B show reduction of blood pressure in patients with unilateral surgical ablation

FIGS. 39A and 39B illustrate relative size of right and left CB in patients with sympathetically mediated diseases

FIG. 40 illustrates reduction of insulin resistance after unilateral surgical ablation of CB.

FIGS. 41A-D and 42A-D include graphical illustrations of transverse cross sections of the carotid body and the internal and external carotid arteries in the vicinity of the carotid body, with presentations of dimensional data from an anatomical analysis of 50 people.

FIGS. 43A-C include graphical illustrations of a sagittal cross section of a carotid body and common, internal, and external carotid arteries in the vicinity of the carotid body, with presentations of dimensional data from an anatomical analysis of 50 people.

### DETAILED DESCRIPTION

The disclosure is related to methods, devices, and systems for planning for, and optionally assessment of, the effective and safe full or partial ablation of one or both carotid bodies, carotid body (“CB”) chemoreceptors, or carotid body nerves to treat patients having a sympathetically mediated cardiac, metabolic, and pulmonary disease (e.g. hypertension, CHF, diabetes, sleep disordered breathing) resulting from peripheral chemoreceptor hypersensitivity, carotid body hyperactivity, high carotid body afferent nerve signaling or heightened sympathetic activation.

The disclosure includes integral assessment for an ablation procedure (e.g., carotid body ablations, and other procedures for treating sympathetically mediated disease), which can include at least one of pre-procedural assessment, analysis and planning, intra-operative measures of technical success, and post-procedural follow-up. The pre-procedural assessment and analysis may include methods for: patient screening, procedural planning, and intra-operative guidance.

Carotid Body Ablation (“CBA”) as used herein refers generally to ablation of a target tissue wherein the desired effect is to reduce or remove the afferent neural signaling from a chemosensor (e.g. carotid body) or reducing a chemoreflex. To inhibit or suppress a peripheral chemoreflex, anatomical targets (also referred to as targeted tissue, target ablation sites, or target sites) may include at least a portion of at least one carotid body, nerves associated with a peripheral chemoreceptor (e.g. carotid body nerves, carotid sinus nerve, carotid plexus), small blood vessels feeding a peripheral chemoreceptor, carotid body parenchyma, chemosensitive cells (e.g. glomus cells), tissue in a location where a carotid body is suspected to reside (e.g. a location based on pre-operative imaging or anatomical likelihood), an intercarotid septum, a substantial part of an intercarotid septum or a combination thereof. As used herein, ablation of a carotid body may refer to ablation of any of these target ablation sites. Exemplary methods, devices, and systems for performing CBA procedures are described below.

With respect to CBA, pre-procedural testing and planning includes identification of patients likely to benefit from CB-targeted therapy and can also include the exclusion of patients that are contraindicated or who demonstrate unacceptable procedural risks. A CBA procedure can involve unilateral CB ablation (i.e., ablating only one of a patient's carotid bodies) or bilateral CB ablation (i.e., ablating two of a patient's carotid bodies). Pre-procedural testing may be performed with respect to a patient's left or right carotid bodies and may be used to determine which CB is suitable, or preferred, for an ablation procedure in the case of a unilateral CB ablation. Patient selection and procedural planning are intended to ensure the highest risk benefit ratio for each individual patient. Pre-procedural screening, based on the results of combined medical tests, may be applied to select patients most likely to benefit from the CBA treatment procedure. Thus, the pre-procedural screening improves the efficacy and safety of a CBA procedure.

Effective pre-procedural screening of patients and/or CBA planning is expected to enhance the success and safety of a CBA procedure. The patient selecting can include selecting patients that are one or more of the following: likely to benefit from the CBA procedure, such as having elevated sympathetic tone, high chemosensitivity, and other clinical indicators that the patient is suitable for CBA; suitable for an endovascular CBA procedure, such as having an identifiable carotid body in a favorable anatomic position; may incur acceptable procedural risks, such as vascular anatomy that allows access to the CB without significant atherosclerotic or arteriosclerotic disease in positions vulnerable to endovascular access.

Further, the pre-procedural planning may give a physician an indication of probability of success or failure of a proposed CBA procedure(s).

The pre-procedural assessment and analysis (which may also herein referred to herein as testing, process, or any such similar term) may include medical tests to improve efficacy and safety of an endovascular CBA procedure to treat sympathetically mediated diseases (e.g. hypertension, congestive heart failure (CHF), diabetes, and insulin resistance renal failure). The tests used in the pre-procedural may be unfamiliar to a typical interventional cardiologist, who is generally expected to perform a CBA procedure. For example, measuring respiration in response to inhaled or injected stimulus is not conventionally associated with treatment of sympathetically mediated diseases or the selection of such patients for treatment of such diseases. While tests to determine the anatomy of the carotid artery are familiar to cardiologists in relation to carotid stenting they are not familiar with respect to ablation procedures. Further, it would have been counterintuitive to a cardiologist performing carotid stenting to select patients with lesser atherosclerosis and arteriosclerosis of the carotid artery.

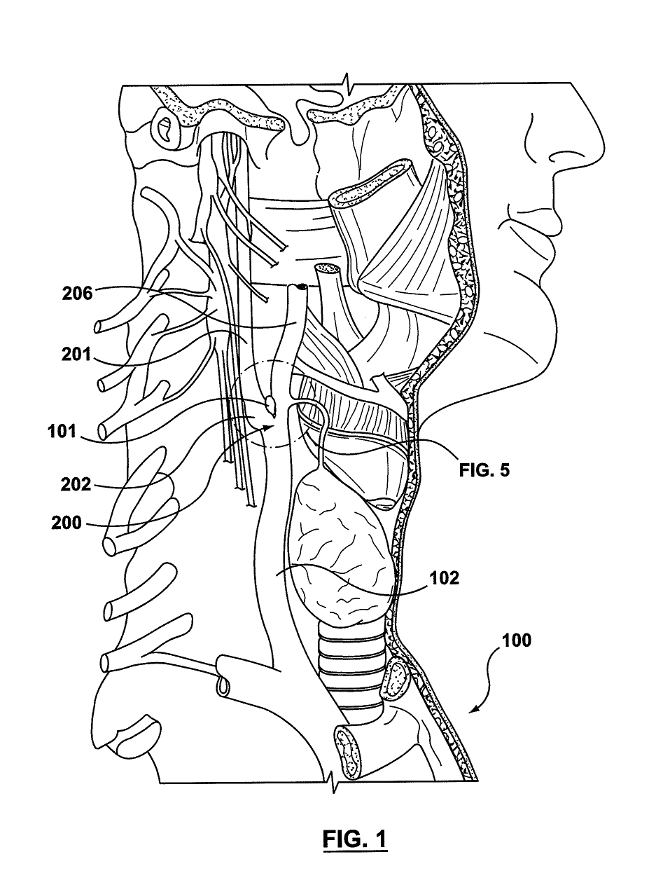

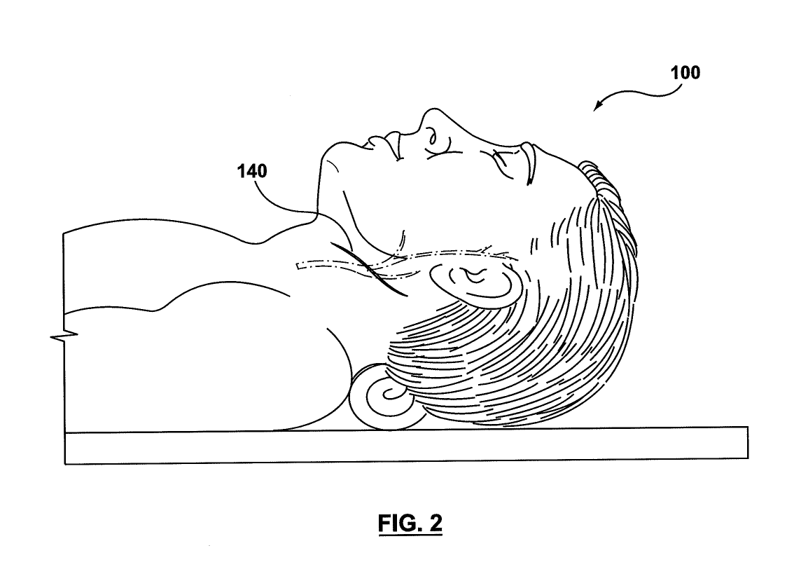

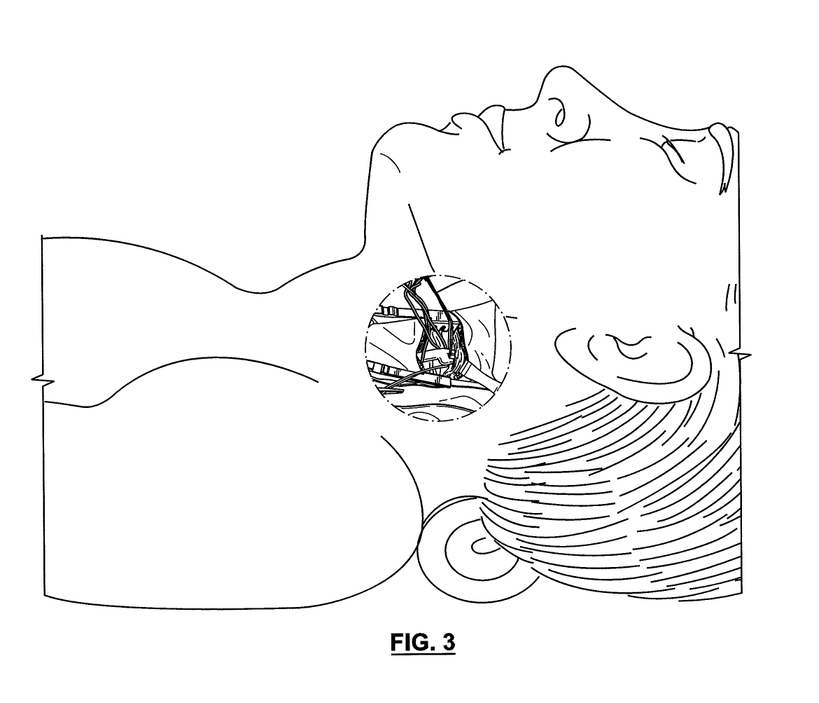

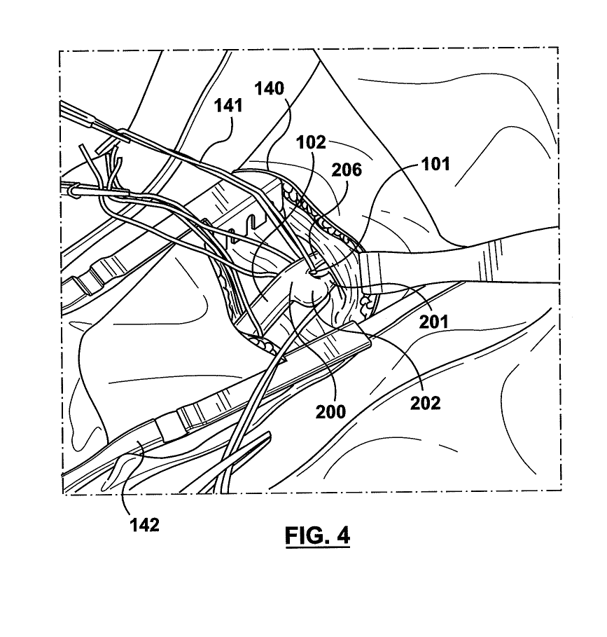

The exemplary methods, and FIGS. 1-6, described in U.S. Provisional Application No. 61/616,897, filed Mar. 28, 2012 are incorporated by reference herein. Any of the steps of the methods herein can be performed with a processing component, and can be included in software algorithm.

Additionally, procedure planning can include imaging of the CB. For example, CB size may be used to identify patients with elevated chemosensitivity. Table C shows data obtained by the authors through a retrospective computed tomography angiography (CTA) study. The results show that a CB is significantly enlarged in patients with sympathetically mediated diseases.

TABLE C
CB size
Normal vs. CHF (P < 0.05)

| | CHF | Normal |
|---|---|---|
| | (N = 36) | (N = 237) |
| Mean (mm) | 2.58 | 2.26 |
| Variance (mm) | 0.61 | 0.33 |

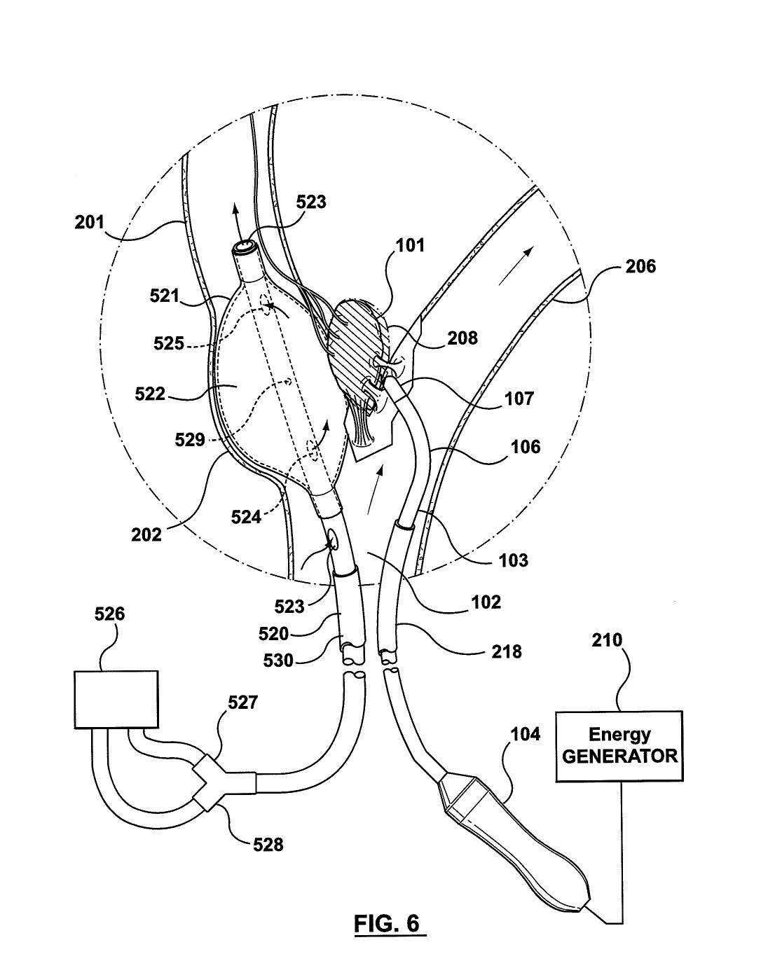

FIG. 6 in U.S. Prov. Application No. 61/616,897, filed Mar. 28, 2012, which is incorporated by reference, illustrates an exemplary flow chart for a process (e.g., an automated process) to capture images of a carotid body or related vasculature, analyze the images and generate assessments as to the suitability of the patient for CBA, procedural suggestions (e.g. medical device configuration such as diameter, length, number of bends and bend radii, torque, flexibility and electrode position; energy delivery parameters or algorithm, approach such as percutaneous, endovascular, or surgical), or risk analysis. Analyzing CB images may be performed using a CB imaging and procedural planning package. The planning package may be an automated process, e.g., a data analysis engine or module, executed by a computer.

A detailed characterization of the patient anatomy can be performed and particularly of the carotid body, related vasculature (e.g. common carotid artery, internal and external carotid arteries, and/or aortic arch), relative anatomical position (e.g., proximity to jaw line which may indicate proximity to a hypoglossal nerve), location of nerves (e.g., vagus or hypoglossal nerve), or pathological conditions of anatomy (e.g. presence, location, and composition of plaque in vessels). The detailed characterization of anatomy of a carotid body is used to determine suitability, from technical and procedural perspectives, of the proposed CBA therapy. Patient suitability can be assessed based on a combination of imaging techniques and analysis.

The imaging techniques used to obtain imaging data on the patient and particularly the carotid body may include electrical mapping, magnetic resonance imaging (MRI), angiography, ultrasound, or computer tomography angiography (CTA). Using a combination of different imaging modalities increases sensitivity and specificity of our proposed method and system to visualize the CB and its surrounding tissues and gather information to plan the procedure. Each of the imaging modality has its distinct strength and is used to image or analyze a particular part or tissue of the target region for its own. A specialized method of CTA, devised by the authors to enhance identification and visualization of a CB, may involve acquiring data during an arterial phase of contrast material passage. Optimizing CTA parameters may include adjustment of contrast injection volume, injection rate, and delay to scan. MRI uses specific scan sequences to highlight relevant anatomy. A specialized method of MRI may be used to enhance identification and visualization of a CB. For example, a CB may be enhanced by highlighting a tissue type or local perfusion. Further, resolution and contrast may be enhanced by using a local coil (carotid coil), using a more powerful magnet, and averaging from multiple scans. Even small motion artifacts due to throat movements can distort MR imaging. Tracking movement of the laryngeal prominence (that is, Adam's Apple) can be used to exclude images taken when throat is moving. MR sequences that may be optimized to visualize CB include: T1 weighted scan, T2 weighted scan, velocity encoded scan (CINE), diffusion weighted imaging, or contrast enhanced imaging (e.g., gadolinium). Pre-procedural imaging may be performed with use of a neck brace that aligns the patients head in a stable and defined direction (e.g. straight, rotated to one side or another by a defined angle). The use of a brace during pre-procedural imaging may allow the patient to be placed in the same configuration during a CBA procedure so pre-procedural images accurately represent anatomical positions of important structures (e.g. CB, carotid arteries, nerves, aortic arch). Additionally, the neck brace may comprise fiducial markers visible on pre-procedural imaging, which may be seen on imaging during a procedure (e.g. with fluoroscopy, or CT).

The planning package can facilitate exclusion of patients from CBA based on an unfavorable anatomy in the working region, such as CB location and the aortic and carotid artery anatomy. Similarly, the planning package may suggest technical parameters, e.g., device, energy to be applied for ablation and vascular approach, for the CBA procedure. Further, the planning package may generate estimated risks associated with the CBA procedure to be performed on the patient, which may exclude a patient from CBA or suggest an appropriate type of CBA procedure to avoid or mitigate the risk.

The planning package can analyze the image data to identify the carotid body and its location in a patient. The analysis can be partially automated and partially performed manually, or it some embodiments it can be performed manually.

The planning package can generate a recommendation as to whether a patient is suitable for CBA based, in part, on whether the imaging data clearly shows the carotid body and its location. A clear image of the carotid body and accurate position information may assist a physician in performing a CBA procedure. CTA, MRI and electrical mapping guided imaging may aid the physician to: (i) localize the CB in the neck region of the patient; (ii) measure distances and angles from a vessel lumen to a target area, such as the CB or CB nerves, and (iii) create a digital map, e.g. 3-dimensional map, of the area proximate to the CB or CB nerves where an ablation catheter is to be positioned.

A combination of CTA, MRI and electrical mapping may provide information to determine the exact location of a CB in the area of a patient's carotid arteries. The planning package may provide essential information on CB size, distances of CB to vessel lumen of adjacent arteries, identify a feeding artery of a CB and characterize the anatomy of the neck region including arteries, veins and nerves. This information can be integrated.

The planning package may use data from two, three or more imaging sources, e.g., electrical mapping, MRI, CTA, ultrasound and angiography, to map and characterize the CB region of a patient. The mapping and characterization of a CB region may be performed partially or fully by the automation of the package. By using data from multiple imaging, the planning package can achieve high precision in mapping and characterizing the CB region. This high precision enables the physician to more accurately position the device, e.g., ablation catheter, in the CB region and thereby improve the safety and efficacy of a CBA procedure.

The planning package evaluates the imaging data to determine if the patient should be a candidate for CBA and, for suitable patients, generates a plan for the CBA process and a navigation map for the device to perform the ablation. Once a patient has been identified as a potential candidate, planning package may be used to define the type of CBA treatment suitable for each patient.

The planning package may analyze image data from several imaging modalities such as: CTA, fluoroscopy, ultrasound, electrical mapping and MRI. This aggregate of imaging data is used, for example, to decide whether endovascular, extravascular or surgical CBA is appropriate. Once chosen, the package can be used to plan and guide the CBA procedure.

In some embodiments the planning package determines the suitability of a patient for an endovascular CBA based one or more of the following exemplary parameters: characteristics of atherosclerosis and arteriosclerosis of a vessel involved in the procedure, (e.g. composition of plaque near the CB or in carotid arteries); localization of atherosclerosis and arteriosclerosis and analysis of its implication for the procedure; risk of complications (e.g., patients with an unacceptably high risk of embolic events, vessel trauma, and the like may be excluded from an endovascular CBA treatment but may be suggested for a surgical or percutaneous CBA treatment); cerebrovascular reserve, e.g., patients with limited cerebrovascular reserve may be excluded from CBA treatment; analysis of adjacent anatomical structures which may need to be preserved (e.g., hypoglossal nerve and/or vagal nerve). Prior to or during a CBA procedure imaging of such structures may be complimented by stimulation to ensure safety. For example, hypoglossal response may be monitored by motoric activation of tongue muscles and vagal activation may be monitored by cardiovascular or respiratory responses or coughing.

Based on the pre-treatment planning and assessments described herein, patients that are determined to not be suitable for endovascular CBA can be considered for a surgical approach to carotid body treatment or a percutaneous approach to CBA.

In addition to making an initial determination that a patient is suitable for CBA treatment, the planning package can generate a recommended plan for CBA treatment or a navigational map for the treatment. During the process of planning and mapping, the package can determine that the CB is not clearly imaged or that the plaque or anatomy of the carotid body are not suited for CBA. In these situations, the process can recommend another treatment approach. As part of the planning process, the planning package can recommend exclusion of patients with high procedural risks or potential technical difficulties. By recommending exclusion of patients while attempting to plan and map the CBA treatment, the planning package decreases the rate of complications and increase the rate of safe, effective, and expedient CBA treatments.

The integrated imaging modalities provide the planning package with imaging data to generate a detailed analysis of the relevant CB anatomy. Based on the imaging data, the planning package can determine whether the carotid body is sufficiently clear in the image data, or a manual determination can be made as to the clarity of the image of the carotid body.

The planning package, which may be embodied as software technology, performs automated or semi-automated image registration of data from several imaging modalities including: CTA, fluoroscopy, MRI, ultrasound and electrical mapping. This creates a reconstructed roadmap of the relevant anatomy, such as a three-dimensional image modeling the carotid body and the external and internal carotid arteries proximate to the carotid body. The image data may be used by the planning package to suggest to the physician a procedural plan and may also allow the physician to explore the target region and prepare the navigation of the upcoming procedure. The integrated plan and map can devise the easiest way to guide a physician manipulating an endovascular catheter to the target region in a manner that minimizes potential complications.

The planning package extracts morphological and compositional indices of atherosclerotic plaque severity, progression, and vulnerability. Precise anatomical measurements (e.g. sizes of relevant arteries, location and characteristics of plaque, angle of bifurcation, size and location of CB, blood flow in relevant vessels) allow for the optimal positioning of an ablation catheter to a target region (e.g., close to a CB with correct angulation of an energy delivery element or ablation element).

Integration of this information allows the planning process to suggest an appropriate type of energy delivery system (e.g. RF, ultrasound, laser, chemical ablation), or energy delivery parameters (e.g. power, duration, slope, temperature) of an ablation catheter.

The planning package may perform comprehensive integration of analysis from multiple imaging modalities, and results from computational modeling, in vitro, in vivo and in situ experimentation may be used algorithmically to define optimal ablation parameters for CBA tailored for each CB in every patient. This can be based on, for example, size of CB and the influence of cooling due to blood flow. Similarly, the planning package may automatically generate a recommendation for energy delivery modality and energy delivery parameters.

As part of procedural planning the planning package may involve the application of ultrasound, CTA, MRI, electrical mapping and angiogram. In addition to functions already described, the planning package may perform the following functions: (i) identify aortic and carotid anatomy (e.g. height of carotid bifurcation, vascular tortuosity, angles of branching vessels, distances);(ii) assess whether the patient is suited for endovascular CBA treatment; (iii) exclude patients which show complicated anatomy or problematic plaque conditions, and thus impose an increased procedural risk; (iv) guide the physician in a procedural approach and tool selection in order reach the target region; (v) quantify blood flow in a target region and target vessel (e.g. Sonography, MRI, Angiography); (vi) analyze tissue composition and distances from a vascular lumen to a CB and adjacent structures; (vii) help the physician choose necessary procedural equipment with the appropriate parameters of energy delivery to the tissue; and (viii) identify baroreflex, vagus nerve and hypoglossal nerve such as based on the electrical mapping or ultrasound from the image data.

In some embodiments the planning package generates a plan and navigation map for the CBA treatment. The physician then performs the planned CBA treatment. After the CBA treatment, the patient may be tested to evaluate chemoreflex response (e.g. chemosensitivity) or other condition to determine if the treatment was successful.

The navigation map that can be generated by the planning package provides data to the physician such as, for example without limitation, the position of the carotid body with respect to the bifurcation of the internal and external carotid arteries, the distance of the carotid body to the walls of the arteries, the angle between the carotid body and a center of each of the arteries, and the diameter of the vessels of the internal and external carotid arteries.

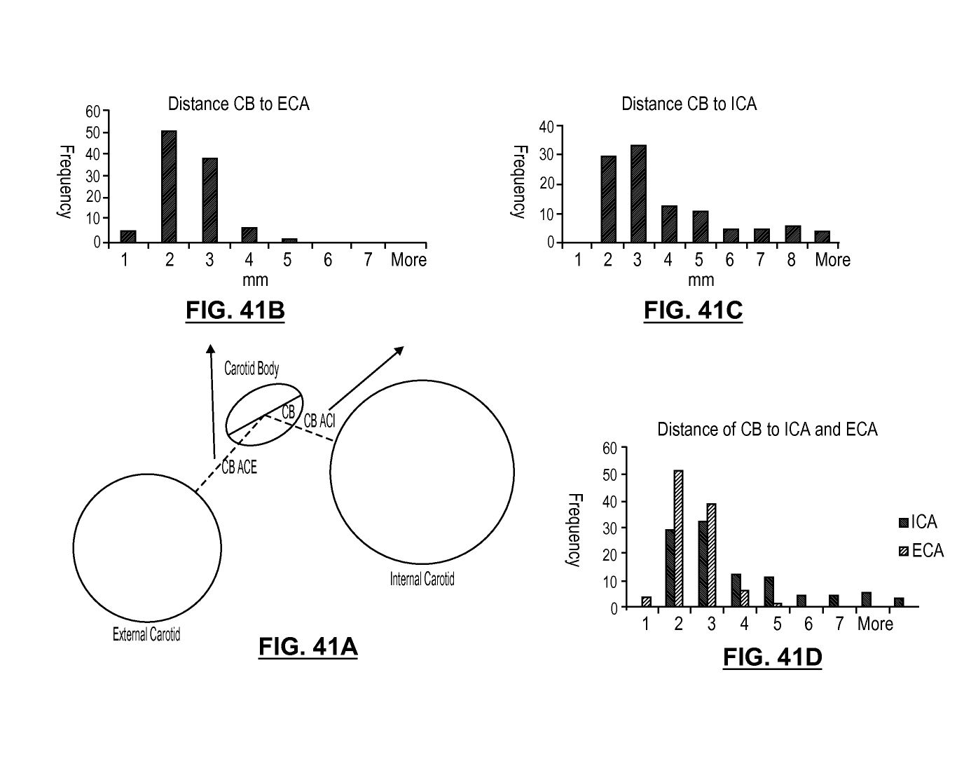

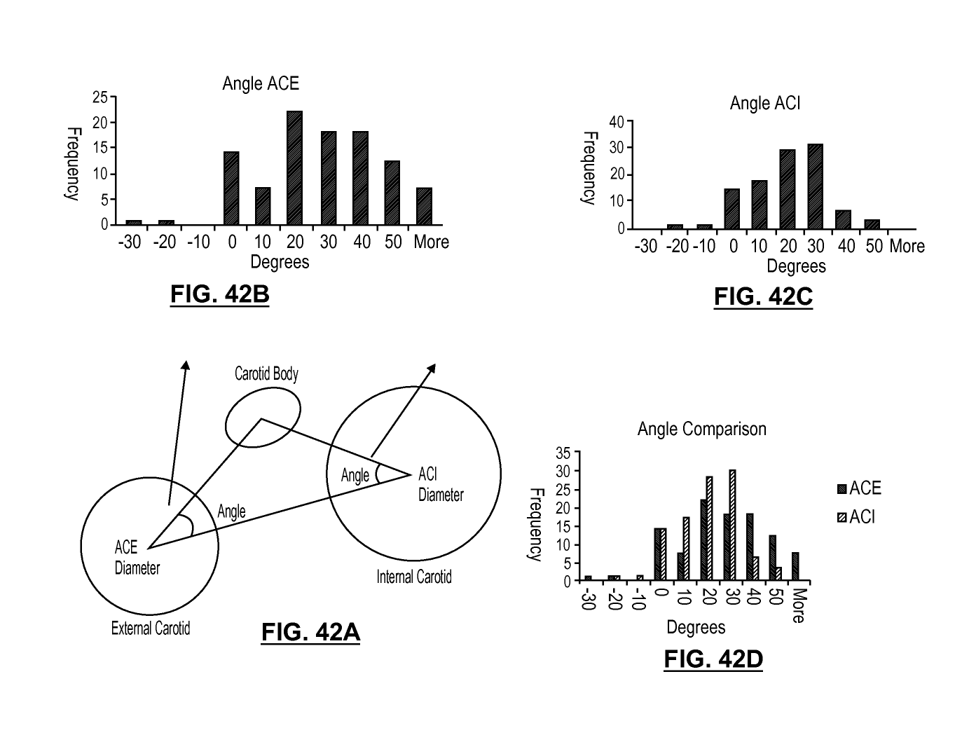

Examples of anatomical analysis of anatomy of a target region including a distribution of significant anatomical parameters for fifty subjects are illustrated in FIGS. 41-43.

FIGS. 41A-41D and 42A-42D include graphical illustrations of geometrical dimensions taken from transverse cross sectional CTA views of carotid bodies and internal and external carotid arteries in the vicinity of the carotid bodies for 50 subjects in a retrospective study along with histograms showing the distribution of data for the study. FIG. 41A illustrates and identifies geometrical dimensions including cross sectional ovoid length of CB, distance from a center of a CB to a nearest edge of an internal lumen of an external carotid artery (illustrated in FIG. 41A as CB-ACE, and shown in FIG. 41B), and distance from the center of a CB to a nearest edge of an internal lumen of an internal carotid artery (illustrated in FIG. 41A as CB-ACI, and shown in FIG. 41C).

FIGS. 42A-42D show geometrical dimensions including angles between lines drawn from center points of internal and external carotid arteries and the CB. The illustrations help to show the distribution of data in a population of the distance and position of carotid bodies relative to internal and external carotid arteries. Automated or manual imaging analysis can thus incorporate this information to identify a carotid body and internal and external carotid arteries.

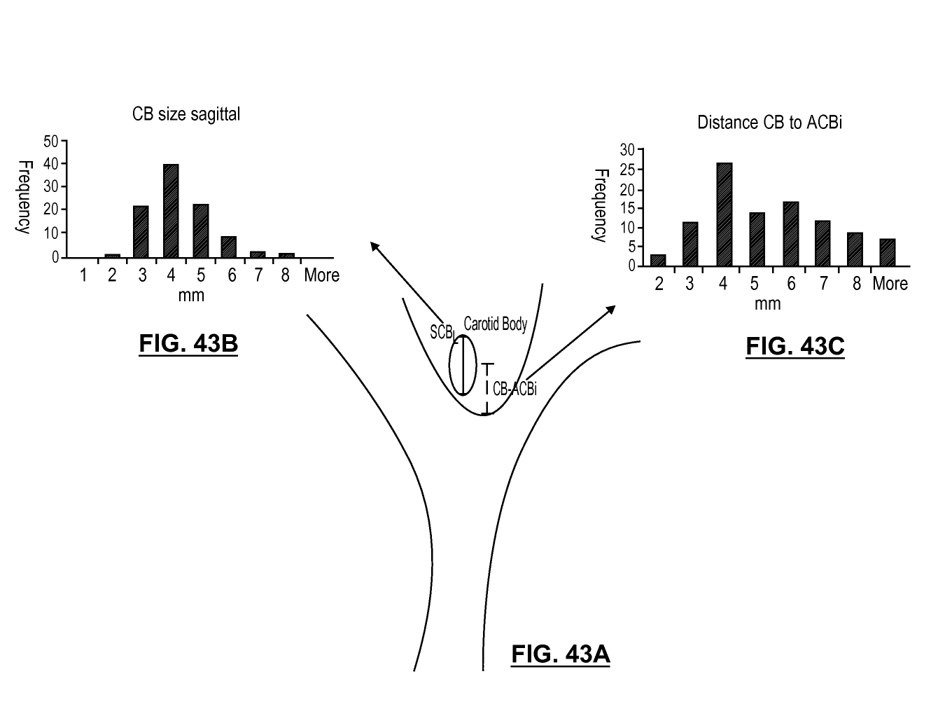

FIGS. 43A-43C illustrate geometrical dimensions including sagittal cross sectional ovoid length of CB (SCBL) and a vertical distance from a center point of the CB to the inner luminal surface of the carotid bifurcation (ACBi) for 50 subjects in a retrospective study along with histograms in FIGS. 43B and 43C showing the distribution of data for the study.

The pre-treatment planning and screening process can also include assessing the size of the carotid body. There is a considerable amount of scientific evidence to demonstrate that hypertrophy of a carotid body often accompanies disease. A hypertrophied or enlarged carotid body may further contribute to the disease. Thus identification of patients with enlarged carotid bodies may be instrumental in determining candidates for therapy.

Further, it is possible that although patients do not meet a preselected clinical or physiological definition of high peripheral chemosensitivity (e.g. greater than or equal to about two standard deviations above normal), administration of a substance that suppresses peripheral chemosensitivity may be an alternative method of identifying a patient who is a candidate for the proposed therapy. These patients may have a different physiology or co-morbid disease state that, in concert with a higher than normal peripheral chemosensitivity (e.g. greater than or equal to normal and less than or equal to about 2 standard deviations above normal), may still allow the patient to benefit from carotid body ablation. The proposed therapy may be at least in part based on an objective that carotid body ablation will result in a clinically significant or clinically beneficial change in the patient's physiological or clinical course. It is reasonable to believe that if the desired clinical or physiological changes occur even in the absence of meeting the predefined screening criteria, then therapy could be performed.

In general, the main pathway of ablation treatment described herein is a reduction of peripheral chemosensitivity, carotid body hyperactivity, or reduction of afferent nerve signaling from the carotid body (CB) resulting in a reduction of central sympathetic tone. Other therapeutic benefits such as reduction of dyspnea, hyperventilation, respiratory alkalosis, periodic breathing and breathing rate may be achieved in some patients. Specifically patients with asthma, COPD and CHF that suffer from dyspnea may benefit from carotid body ablation through the reduction of debilitating symptoms and increase of ability to exercise independently from their sympathetic nerve activity. Benefits of exercise are well known in CHF and COPD patients and dyspnea at exertion or (in severe cases) at rest is a major impediment to healthy lifestyle.

The terms “ablate” and “ablation” may refer to the act of altering a tissue to suppress or inhibit its biological function or ability to respond to stimulation. For example, ablation may involve, but is not limited to, thermal necrosis (e.g. using energy such as thermal energy, radiofrequency electrical current, direct current, microwave, ultrasound, high intensity focused or not focused ultrasound, and laser), cryogenic ablation, electroporation, selective denervation (e.g. destruction of active, sensing and conducting tissues inside the carotid septum, chemosensitive cells, afferent nerves from the carotid body while preserving nerves from the carotid sinus which conduct baroreceptor signals) and other adjacent nerves and important anatomic structures proximate to the carotid septum. The term “Ablation” may refer to the act of altering a tissue to suppress or inhibit its biological function or ability to respond to stimulation permanently or for an extended period of time (e.g. greater than 3 weeks, greater than 6 months, greater than a year, for several years, or for the remainder of the patient's life). Ablation can mean selective denervation and may involve, for example, interruption of afferent nerves from a carotid body while substantially preserving non-target nerves such as sympathetic trunk nerves, vagus, glossopharyngeal, or hypoglossal nerves. Another example of selective denervation may involve interruption of a carotid sinus nerve, or intercarotid plexus which is in communication with both a carotid body and some baroreceptors wherein chemoreflex from the carotid body is reduced permanently or for an extended period of time (e.g. years) and baroreflex is substantially restored in a short period of time (e.g. days or weeks).

Carotid body ablation may at least in part be due to alteration of vascular or peri-vascular structures (e.g. arteries, arterioles, capillaries or veins), which perfuse the carotid body and neural fibers surrounding and innervating the carotid body (e.g. nerves that transmit afferent information from carotid body chemoreceptors to the brain). Additionally or alternatively ablation may include tissue disruption due to a healing process, fibrosis, or scarring of tissue following injury, particularly when prevention of regrowth and regeneration of active tissue is desired.

Ablation may be induced by delivering ablation energy such as thermal energy (heat or cold), chemical ablation, mechanical energy, or electrical energy. Examples of ablation energy modalities include radiofrequency, cryogenic, microwave, ultrasound, high intensity ultrasound, low frequency ultrasound, surgical resection, or minimally invasive surgical resection. Approaches for ablation may include endovascular, percutaneous, or extracorporeal.

The treatment may involve inserting a catheter in the patient's vascular system, positioning an energy delivery element at the distal end of the catheter proximate to chemoreceptors and delivering ablative energy to the chemoreceptors in order to ablate them. Other methods and devices for chemoreceptor ablation are described. Positioning an energy delivery element at the distal end of the catheter proximate to chemoreceptors may involve positioning the energy delivery element at the bifurcation of carotid artery and ablating tissue contained within the carotid septum.

The devices, systems, and methods for carrying out a full or partial ablation therapy herein are merely exemplary. The disclosure includes methods and devices for planning and preparing for an ablation therapy, and is not limited to the specific devices and methods for carrying out the therapy described herein.

As shown in FIG. 1, the carotid body 101, a small, ovoid-shaped (often described as a grain of rice), and highly vascularized organ is situated in or near the carotid bifurcation 200, where the common carotid artery 102 branches in to an internal carotid artery (IC) 201 and external carotid artery (EC) 206. The central chemoreceptors are sensitive to hypercapnia (high PCO₂), and the peripheral chemoreceptors are sensitive to hypercapnia and hypoxia (low blood PO₂). Under normal conditions activation of the sensors by their respective stimuli results in quick ventilatory responses aimed at the restoration of cellular homeostasis.

To inhibit or suppress a peripheral chemoreflex, anatomical targets for ablation (e.g. targeted tissue or target sites) may include at least one carotid body, an aortic body, nerves associated with a peripheral chemoreceptor, small blood vessels feeding a peripheral chemoreceptor, carotid body parenchyma, chemosensitive cells (historically called glomus cells), or a combination thereof.

Ablation is to be focused on targeted tissue, or be focused on the targeted tissue while safely ablating tissue proximate to the targeted tissue (e.g. to ensure the targeted tissue is ablated or as an approach to gain access to the targeted tissue). The targeted tissue for ablation may be as big as the peripheral chemoreceptor (e.g. carotid body or aortic body) itself, somewhat smaller, or bigger and can include tissue surrounding the chemoreceptor such as blood vessels, adventitia, fascia, small blood vessels perfusing the chemoreceptor, or nerves connected to and innervating the chemosensitive cells.

Methods and devices have been conceived by the inventors comprising, in a selected patient, advancing an endovascular catheter into a common carotid artery proximate a carotid septum, positioning ablative energy delivery elements on the carotid septum, and applying ablative energy such that contents of the carotid septum are substantially ablated while tissues outside of carotid septum are substantially preserved, wherein endovascular ablation of the carotid septum (e.g. the right, left, or both carotid septa) results in reduced cumulative afferent nerve signals to the brain and reduction or normalization of blood pressure.

Tissue may be ablated to inhibit or suppress a chemoreflex of only one of a patient's two carotid bodies. Another embodiment involves ablating tissue to inhibit or suppress a chemoreflex of both of a patient's carotid bodies. For example a therapeutic method may include sequential ablation of one carotid body and not the other, measurement of resulting chemosensitivity and ablation of the second carotid body if needed to further reduce chemosensitivity following unilateral ablation.

Investigators in Wroclaw and Gdansk hospitals in Poland performed surgical ablation of right carotid bodies in patients with CHF, HTN, Insulin Resistance and Diabetes under direction of the Inventors. Observed improvements in exercise tolerance, glucose metabolism and particularly in blood pressure exceeded expectations of investigators and Inventors at one month and up to six months after surgery in some patients. In all of the surgical cases the right side carotid body was ablated (by surgical resection and carotid septum excision). The choice of right side carotid body was justified by the potentially less severe complications in the event of accidental embolization of the brain by debris. In addition, the right carotid body in most humans tends to be larger. For these reasons Inventors propose that endovascular trans-catheter carotid body ablation is also performed on the right side. If the ablation of right carotid body is insufficient to reach the desired clinical goals (e.g. normalization of blood pressure), the left carotid body may also be ablated.

The ablation procedure is targeted on the carotid body to substantially reduce chemoreflex without substantially reducing the baroreflex of the patient or damaging adjacent nerves that control facial or throat muscles such as for example pharyngeal and laryngeal nerves. The proposed ablation procedure may be targeted to substantially spare the carotid sinus, baroreceptors distributed in the walls of carotid arteries (specifically internal carotid artery), and at least some of the carotid sinus nerves that conduct signals from said baroreceptors. For example, the baroreflex may be substantially spared by targeting a limited volume of ablated tissue referred to herein as the carotid septum which encloses the carotid body, tissues containing a substantial number of carotid body nerves, tissues located in adventitia of a medial segment of a carotid bifurcation, tissue located at the attachment of a carotid body to an artery, or extending to tissues located on the medial side of a carotid artery bifurcation saddle and avoiding damage to the lateral side. Targeting the tissue to be ablated may be enabled by visualization of the tissue area or of the carotid body itself, for example by CT, ultrasound sonography, fluoroscopy, blood flow visualization, or injection of contrast. The visualization of the tissue area may be used to aid the positioning of a distal region of a catheter in the carotid body or in close proximity while avoiding excessive damage (e.g. perforation, stenosis, thrombosis) to carotid arteries, baroreceptors or carotid sinus nerves. Imaging a carotid body before ablation may be instrumental in (a) selecting candidates if CB is present, large enough and identified and (b) guiding therapy by providing a landmark map for an operator to guide an ablation instrument to carotid body nerves, the area of a blood vessel proximate to a carotid body, or to an area where carotid body nerves may be anticipated.

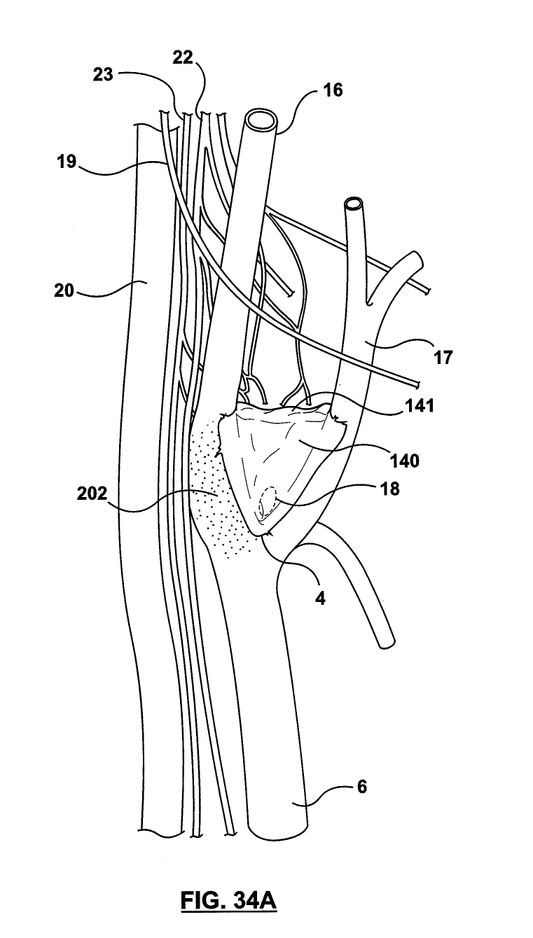

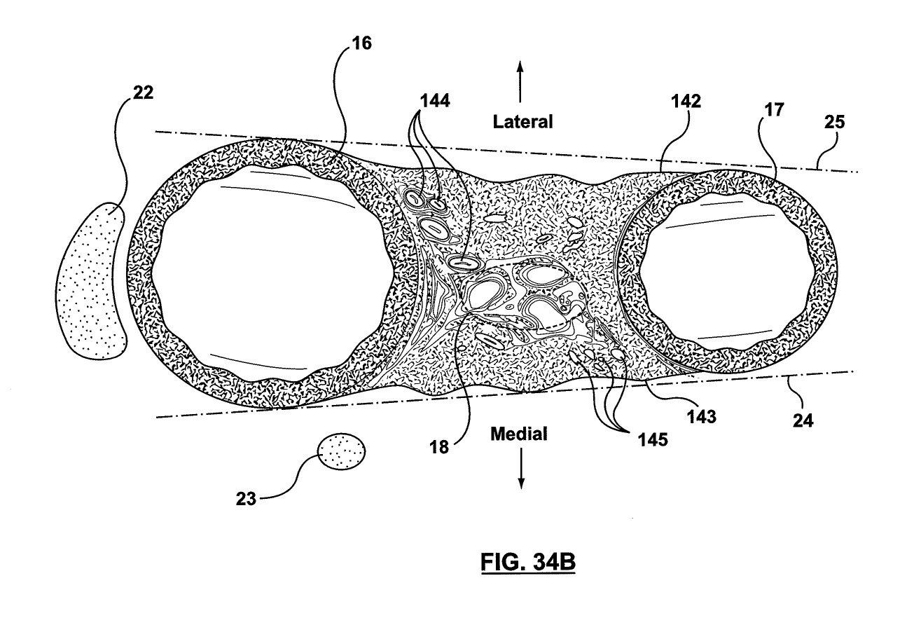

An intercarotid septum 140 (also referred to as carotid septum) shown in FIGS. 34A and 34B is herein defined as a wedge or triangular segment of tissue with the following boundaries: A saddle of a carotid bifurcation 4 defines a caudal aspect (an apex) of a carotid septum 140; Facing walls of internal 16 and external 17 carotid arteries define two sides of a carotid septum; A cranial boundary 141 of a carotid septum extends between these arteries and may be defined as cranial to a carotid body but caudal to any vital nerve structures (e.g. hypoglossal nerve) that might be in the region, for example a cranial boundary may be about 10 mm (possibly 15 mm) from the saddle of the carotid bifurcation 4; Medial 142 and lateral 143 walls of the carotid septum 140 are generally defined by planes approximately tangent to the internal and external carotid arteries; One of the planes is tango the lateral wall of the internal and external carotid arteries and the other plane is tangent to the medial walls of these arteries. An intercarotid septum is between medial and lateral walls. An intercarotid septum 140 may contain a carotid body 18 and may be absent of vital structures such as a vagus nerve 22 or vital sympathetic nerves 23 or a hypoglossal nerve 19. An intercarotid septum may include some baroreceptors 202 or baroreceptor nerves. An intercarotid septum may also include various nerves of intercarotid plexus, small blood vessels 144 and fat 145.

Carotid body nerves are anatomically defined herein as carotid plexus nerves 144 and carotid sinus nerves. Carotid body nerves are functionally defined herein as nerves that conduct information from a carotid body to a central nervous system.

Ablation may be focused exclusively on targeted tissue, or be focused on the targeted tissue while safely ablating tissue proximate to the targeted tissue (e.g. to ensure the targeted tissue is ablated or as an approach to gain access to the targeted tissue). An ablation may be as big as the peripheral chemoreceptor (e.g. carotid body or aortic body) itself, somewhat smaller, or bigger and can include tissue surrounding the chemoreceptor such as blood vessels, adventitia, fascia, small blood vessels perfusing the chemoreceptor, or nerves connected to and innervating the chemosensitive cells. An intercarotid plexus or carotid sinus nerve maybe a target of ablation with an understanding that some baroreceptor nerves will be ablated together with carotid body nerves. Baroreceptors are distributed in the human arteries and have high degree of redundancy.

Tissue may be ablated to inhibit or suppress a chemoreflex of only one of a patient's two carotid bodies. Another embodiment involves ablating tissue to inhibit or suppress a chemoreflex of both of a patient's carotid bodies. For example a therapeutic method may include ablation of one carotid body, measurement of resulting chemosensitivity, sympathetic activity, respiration or other parameter related to carotid body hyperactivity and ablation of the second carotid body if needed to further reduce chemosensitivity following unilateral ablation.

Limiting the ablation to the carotid septum should substantially reduce chemoreflex without excessively reducing the baroreflex of the patient. The ablation procedure is targeted to substantially spare the carotid sinus, baroreceptors distributed in the walls of carotid arteries (specifically internal carotid artery), and at least some of the carotid sinus nerves that conduct signals from said baroreceptors. For example, the baroreflex may be substantially spared by targeting the carotid septum to ablate tissue enclosing the carotid body, tissues containing a substantial number of carotid body nerves, tissues located in periadventitial space of a medial segment of a carotid bifurcation, or tissue located at the attachment of a carotid body to an artery.

The tissue to be targeted for ablation may be visualized by CT, CT angiography, MRI, ultrasound sonography, fluoroscopy, blood flow visualization, or injection of contrast, and positioning of an instrument in the carotid body or in close proximity while avoiding excessive damage (e.g. perforation, stenosis, thrombosis) to carotid arteries, baroreceptors, carotid sinus nerves or other vital nerves such as vagus nerve or sympathetic nerves located primarily outside of the carotid septum. Imaging a carotid body before ablation may assist in (a) selecting patients having a carotid body that is large enough and identified, and (b) guiding therapy by providing a landmark map for an operator to guide an ablation instrument to the carotid septum, center of the carotid septum, carotid body nerves, the area of a blood vessel proximate to a carotid body, or to an area where carotid body itself or carotid body nerves may be anticipated. It may also help exclude patients in whom the carotid body is located substantially outside of the carotid septum in a position close to a vagus nerve, hypoglossal nerve, jugular vein or some other structure that can be endangered by ablation. In one embodiment only patients with carotid body substantially located within the intercarotid septum are selected for ablation therapy.

Once a carotid body is ablated, removed or denervated, the carotid body function (e.g. carotid body chemoreflex) does not substantially return in humans (in humans aortic chemoreceptors are considered undeveloped). In contrast, once a carotid sinus baroreflex is removed it is generally compensated, after weeks or months, by the aortic or other arterial baroreceptor baroreflex. Thus, if both the carotid chemoreflex and baroreflex are removed or substantially reduced, for example by interruption of the carotid sinus nerve or intercarotid plexus nerves, baroreflex may eventually be restored while the chemoreflex may not. The consequences of temporary removal or reduction of the baroreflex can be in some cases relatively severe and require hospitalization and management with drugs, but they generally are not life threatening, terminal or permanent. Thus, it is understood that while selective removal of carotid body chemoreflex with baroreflex preservation may be desired, it may not be absolutely necessary and the risk that the baroreflex is removed or reduced need not be a reason to exclude a patient from carotid body ablation.

A carotid body ablation procedure may also comprise a method for assessing a target ablation site, for example to confirm if an ablation device is placed appropriately for safe and effective carotid body ablation. Assessing a target ablation site may comprise temporarily stunning or blocking nerve conduction by cooling to non-ablative temperatures or at non-ablative cooling rates, or via electrical neural blockade. A temporary nerve block may be used to confirm position of an ablation element prior to ablation. For example, a temporary nerve block may block nerves associated with a carotid body, which may result in a physiological effect to confirm the position may be effective for ablation. Furthermore, a temporary nerve block may block vital nerves such as vagal, hypoglossal or sympathetic nerves that are preferably avoided, resulting in a physiological effect (e.g. physiological effects may be noted by observing the patient's eyes, tongue, throat or facial muscles or by monitoring patient's heart rate and respiration). This may alert a user that the position is not in a safe location. Likewise absence of a physiological effect indicating a temporary nerve block of such vital nerves in combination with a physiological effect indicating a temporary nerve block of carotid body nerves may indicate that the position is in a safe and effective location for carotid body ablation. Ablation is a function of time as well as temperature. Thus cooling may be applied to an ablation target site (e.g. carotid body, carotid body nerves or carotid septum) and neural effects may be observed. If undesired neural effects are observed immediately after cooling, ablation can be interrupted while the process of ablation is still in the reversible phase. If only desired effects are observed, cooling can continue maintaining low temperature for a duration long enough to ensure irreversible ablation of affected tissues.

Important nerves may be located in proximity of the carotid septum target site and may be inadvertently and unintentionally injured. These nerves may include the following:

Vagus Nerve Bundle—The vagus is a bundle of nerves that carry separate functions, for example: a) branchial motor neurons (efferent special visceral) which are responsible for swallowing and phonation and are distributed to pharyngeal branches, superior and inferior laryngeal nerves; b) visceral motor (efferent general visceral) which are responsible for involuntary muscle and gland control and are distributed to cardiac, pulmonary, esophageal, gastric, celiac plexuses, and muscles, and glands of the digestive tract; c) visceral sensory (afferent general visceral) which are responsible for visceral sensibility and are distributed to cervical, thoracic, abdominal fibers, and carotid and aortic bodies; d) visceral sensory (afferent special visceral) which are responsible for taste and are distributed to epiglottis and taste buds; e) general sensory (afferent general somatic) which are responsible for cutaneous sensibility and are distributed to auricular branch to external ear, meatus, and tympanic membrane. Dysfunction of the vagus may be detected by a) vocal changes caused by nerve damage (damage to the vagus nerve can result in trouble with moving the tongue while speaking, or hoarseness of the voice if the branch leading to the larynx is damaged); b) dysphagia due to nerve damage (the vagus nerve controls many muscles in the palate and tongue which, if damaged, can cause difficulty with swallowing); c) changes in gag reflex (the gag reflex is controlled by the vagus nerve and damage may cause this reflex to be lost, which can increase the risk of choking on saliva or food); d) hearing loss due to nerve damage (hearing loss may result from damage to the branch of the vagus nerve that innervates the concha of the ear): e) cardiovascular problems due to nerve damage (damage to the vagus nerve can cause cardiovascular side effects including irregular heartbeat and arrhythmia); or f) digestive problems due to nerve damage (damage to the vagus nerve may cause problems with contractions of the stomach and intestines, which can lead to constipation).

Superior Laryngeal Nerve—the superior laryngeal nerve is a branch of the vagus nerve bundle. Functionally, the superior laryngeal nerve function can be divided into sensory and motor components. The sensory function provides a variety of afferent signals from the supraglottic larynx. Motor function involves motor supply to the ipsilateral cricothyroid muscle. Contraction of the cricothyroid muscle tilts the cricoid lamina backward at the cricothyroid joint causing lengthening, tensing and adduction of vocal folds causing an increase in the pitch of the voice generated. Dysfunction of the superior laryngeal nerve may change the pitch of the voice and causes an inability to make explosive sounds. A bilateral palsy presents as a tiring and hoarse voice.

Cervical Sympathetic Nerve—The cervical sympathetic nerve provides efferent fibers to the internal carotid nerve, external carotid nerve, and superior cervical cardiac nerve. It provides sympathetic innervation of the head, neck and heart. Organs that are innervated by the sympathetic nerves include eyes, lacrimal gland and salivary glands. Dysfunction of the cervical sympathetic nerve includes Horner's syndrome, which is very identifiable and may include the following reactions: a) partial ptosis (drooping of the upper eyelid from loss of sympathetic innervation to the superior tarsal muscle, also known as Miller's muscle); b) upside-down ptosis (slight elevation of the lower lid); c) anhidrosis (decreased sweating on the affected side of the face); d) miosis (small pupils, for example small relative to what would be expected by the amount of light the pupil receives or constriction of the pupil to a diameter of less than two millimeters, or asymmetric, one-sided constriction of pupils); e) enophthalmos (an impression that an eye is sunken in); f) loss of ciliospinal reflex (the ciliospinal reflex, or pupillary-skin reflex, consists of dilation of the ipsilateral pupil in response to pain applied to the neck, face, and upper trunk. If the right side of the neck is subjected to a painful stimulus, the right pupil dilates about 1-2 mm from baseline. This reflex is absent in Horner's syndrome and lesions involving the cervical sympathetic fibers)

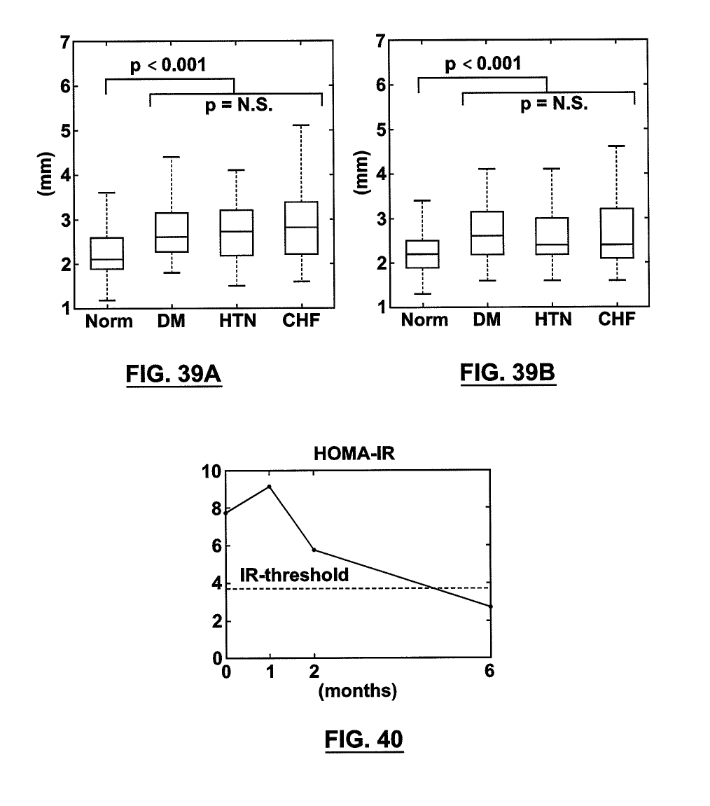

FIGS. 39A and 39B illustrate relative size of right and left carotid bodies in humans with and without sympathetically mediated diseases as observed in a study of approximately 250 CTAs of human neck images performed at the University of Utah at the request of the Inventors. The study measured carotid body size in patients with and without sympathetically mediated diseases. It was found that in patients with diabetes (DM), HTN and CHF the carotid bodies are larger vs. normal patients. Both left and right carotid bodies were found to be larger. The study analyzed CTA images of 76 patients with diabetes (DM), 50 with CHF, 134 with hypertension (HTN) and 124 healthy controls. Results demonstrated that patients with sympathetically mediated diseases have larger carotid bodies than in normal patients and that in both normal and diseased patients the right side carotid body tends to be larger than the left side carotid body.

| Right CB Larger | CBs equal in size | Left CB Larger |
|---|---|---|
| 48.4% | 14.5% | 37.1% |

Based on the knowledge of the carotid body size the following improvements in patient selection and procedure planning can be made. Patients with larger carotid bodies as visualized on CTA can be selected as likely to benefit from carotid body modulation. For example, patients with a carotid body larger than 2.5 mm diameter may be selected. If a patient has a larger right carotid body it may be the first choice to do the right side carotid procedure. If carotid body size is not available, then a default right side procedure may be chosen based on statistics.

Embodiments of the present disclosure may include methods and systems for the transvascular thermal ablation of tissue for the complete or partial ablation of a carotid body via thermal heating or cooling mechanisms to achieve a reduction of carotid body chemoreflex. Several methods disclosed herein form lesions at, or proximate to, the carotid body, which permanently (or for at least an extended period) suppresses or inhibits natural chemoreceptor functioning, which is in contrast to neuromodulating or reversibly deactivating and reactivating chemoreceptor functioning.

Thermally-induced ablation may be achieved via an apparatus positioned proximate to targeted tissue that may include chemoreceptor cells, afferent nerves, or nerve endings (e.g. neural fibers). The apparatus may be, for example, positioned within a carotid artery vasculature (e.g., positioned intravascularly for example in an external carotid artery), positioned extravascularly, positioned intra-to-extravascularly, positioned percutaneously, positioned surgically via incision, or a combination thereof. Thermal destruction of tissue (e.g. thermal ablation) can be achieved by either heating or cooling (e.g. cryo-ablation) and may be due to a direct effect on tissues and structures that are induced by the thermal stress. Additionally or alternatively, the thermal disruption may at least in part be due to alteration of vascular or peri-vascular structures (e.g. arteries, arterioles, capillaries or veins), which perfuse the carotid body and neural fibers surrounding the carotid body (e.g. nerves that transmit afferent information from carotid body chemoreceptors to the brain). Additionally or alternatively thermal disruption may be due to a healing process, fibrosis, or scarring of tissue following thermal injury, particularly when prevention of regrowth and regeneration of active tissue is desired. Common to these thermally-induced ablation procedures the afferent neural signaling from the carotid body is reduced or removed and the chemoreflex sensitivity is reduced, as is generally indicated by a reduction of an increase of ventilation and ventilation effort per unit of blood gas change and by a reduction of central sympathetic nerve activity that can be measured indirectly. Sympathetic nerve activity can be assessed by measuring activity of peripheral nerves leading to muscles (MSNA), heart rate (HR), heart rate variability (HRV), production of hormones such as renin, epinephrine and angiotensin, and peripheral vascular resistance. All these parameters are measurable and can lead directly to the health improvements. In the case of CHF patients blood pH, blood PCO2, degree of hyperventilation and metabolic exercise test parameters such as peak VO2, and VE/VCO2 slope are equally important. It is believed that patients with heightened chemoreflex have low VO2 and high VE/VCO2 slope (index of respiratory efficiency) as a result of tachypnea and low blood CO2. These parameters are also firmly related exercise limitations that further speed up patient's status deterioration towards morbidity and death.

Thermal disruption includes inducement of any mechanism that results in an inability or reduction of ability of the carotid body to transduce or transmit information to the brain (specifically to the brain medulla via the nerve of Hering) or other organs/sensors that result in the negative physiological and clinical events previous noted. This reduction of carotid body ability to transmit information to the brain may be the reduction in tonic nerve activity or response to acute hypoxia or hypoxemia. It is accepted that the carotid body responds primarily to hypoxia but also responds to carbon dioxide, hydrogen ion, blood pH and glucose concentration. It can also manifest as a reduction of response to intermittent, for example nocturnal, hypoxia.

Nerve of Hering is a branch of the cranial nerve IX (glossopharyngeal nerve). The glossopharyngeal nerve synapses in the nucleus tractus solitarius (NTS) located in the medulla of the brainstem. Anatomically the nerve of Hering is a branch of the glossopharyngeal nerve to the carotid sinus and the carotid body. It is the nerve that runs downwards anterior to the internal carotid artery and communicates with the vagus and sympathetic chain and then divides in the angle of bifurcation of the common carotid artery to supply the carotid body and carotid sinus. It carries impulses from the baroreceptors in the carotid sinus, to help maintain a more consistent blood pressure, and from chemoreceptors in the carotid body via separate nerve fibers. It is also known as “Hering's nerve”.

As used herein, thermal heating mechanisms for ablation include both thermal ablation and non-ablative thermal injury or damage (e.g., via sustained heating or resistive heating). Thermal heating mechanisms may include raising the temperature of target neural fibers above a desired threshold, for example, above a body temperature of about 37° C. e.g., to achieve non-ablative thermal injury, or above a temperature of about 45° C. (e.g. above about 60° C.) to achieve ablative thermal injury.

As used herein, thermal-cooling mechanisms for ablation may include reducing the temperature of target neural fibers below a desired threshold (e.g. to achieve freezing thermal injury). It is generally accepted that temperatures below −40° C. applied over a minute or two results in irreversible necrosis of tissue and scar formation.

In addition to monitoring or controlling the temperature during thermal ablation, a length of exposure to thermal stimuli may be specified to affect an extent or degree of efficacy of the thermal ablation. For example, the length of exposure to thermal stimuli may be longer than instantaneous exposure (e.g. longer than about 30 seconds, or even longer than 2 minutes). Furthermore, the length of exposure can be less than 10 minutes, though this should not be construed as the upper limit of the exposure period.

When conducting ablation via thermal mechanisms, the temperature threshold discussed previously may be determined as a function of the duration of exposure to thermal stimuli. Additionally or alternatively, the length of exposure may be determined as a function of the desired temperature threshold. These and other parameters may be specified or calculated to achieve and control desired thermal ablation.

In some embodiments, thermally-induced ablation of glomus cells may be achieved via direct application of thermal cooling or heating energy to the target neural fibers. For example, a chilled or heated fluid can be applied at least proximate to the target, or heated or cooled elements (e.g., thermoelectric element, resistive heating element, cryogenic tip or balloon) can be placed in the vicinity of the glomus. In other embodiments, thermally-induced ablation may be achieved via indirect generation or application of thermal energy to the target neural fibers, such as through application of an electric field (e.g. radiofrequency, alternating current, and direct current), high-intensity focused ultrasound (HIFU), laser irradiation, or microwave radiation, to the target neural fibers. For example, thermally induced ablation may be achieved via delivery of a pulsed or continuous thermal electric field to the target tissue, the electric field being of sufficient magnitude or duration to thermally induce ablation of the target tissue (e.g., to heat or thermally ablate or cause necrosis of the glomus). Additional and alternative methods and apparatuses may be utilized to achieve thermally induced ablation, as described hereinafter.

An embodiment of the present disclosure comprises a surgical technique (e.g. open surgery, laparoscopic, endoscopic, robotically assisted) to gain access to a carotid body or carotid body area. For example, a surgical approach may comprise steps known in the art for surgically accessing a carotid body for glomectomy or tumor removal. As shown in FIGS. 2 to 4, a carotid bifurcation 200 can be exposed through an incision 140 in a patient's skin revealing a common carotid artery 102, an internal carotid artery 201, a carotid sinus 202, an external carotid artery 206, and a carotid body 101. The structures may be identified and restrained with string or clamps to visually expose and stabilize a carotid body 101.

Optionally, prior to obtaining surgical access to a carotid body, non-invasive imaging (e.g. CT, MRI, sonography) of a patient's carotid area may be performed to assess the location and morphology of the carotid body and surrounding structures. The imaging may be used to ascertain risk assessment of surgical access and ablation. It may be used in combination with other patient assessment results (e.g. chemosensitivity) to guide a decision to proceed with a surgical carotid body ablation procedure or not. The imaging may further be used to guide a surgical procedure. For example, an understanding of relative position and morphology of anatomical structures as seen in images may assist a surgeon to obtain surgical access to a carotid body. Furthermore, if a carotid body is difficult to find or is not visible during surgery, images may be used to determine a most probable location of a target site and reduce unnecessary discretion.

Optionally, when the carotid bifurcation is visually exposed a stimulation electrode may be placed in contact with a structure suspected to be a carotid body. The stimulation electrode may be located on the distal tip of a stimulation probe. Alternatively, the stimulation electrode may be located on an ablation device such as a radiofrequency electrode. A stimulation electrode may be an electrically conductive surface (e.g. stainless steel) that delivers stimulation current to tissue in contact. The stimulation electrode may be electrically connected with a wire to a stimulation signal generator. A return electrode (e.g. dispersive electrode patch) may be placed on the patient's skin to complete the electrical circuit. Optionally, the stimulation signal generator may communicate with physiological monitors (e.g. equipment that monitors heart rate, ventilation, blood pressure, blood flow) so a correlation may be made between stimulation and physiological effect. Electrical stimulation may be used to confirm contact with or sufficient proximity to a carotid body and non-contact with or sufficient distance from a baroreceptor prior to ablation, as will be discussed in more detail later.

The carotid body can be ablated using a surgical tool. In one embodiment of a surgical technique a carotid body is tied off tightly and cut off using a scalpel or a harmonic scalpel. An RF heated snare can be also used to reduce bleeding. In another embodiment a carotid body is crushed using a tool such as clamp forceps or cryogenic forceps. In another embodiment a carotid body is ablated using electrocautery forceps. A benefit of electrocautery is control of bleeding and barrier to potential re-innervation and re-growth of the carotid body chemosensitive cells. In another embodiment a carotid body is ablated with RF. An RF electrode on the tip of a probe may be held in contact with, or inserted into the carotid body (or tissue where a carotid body is expected to reside if it is not entirely visible and obscured by fibrous or nerve tissues) while RF energy is applied. In another embodiment a carotid body is ablated using a cryogenic probe. In another embodiment a carotid body may be ablated by applying a cryogen such as liquid nitrogen directly to the carotid body.

Optionally, if there is any doubt that the carotid body was ablated, electrical, mechanical, or chemical stimulation may be used during or following the ablation procedure to confirm ablation. To confirm that baroreflex is intact mechanical, electrical, chemical stimulation of the carotid sinus can be used during the procedure.

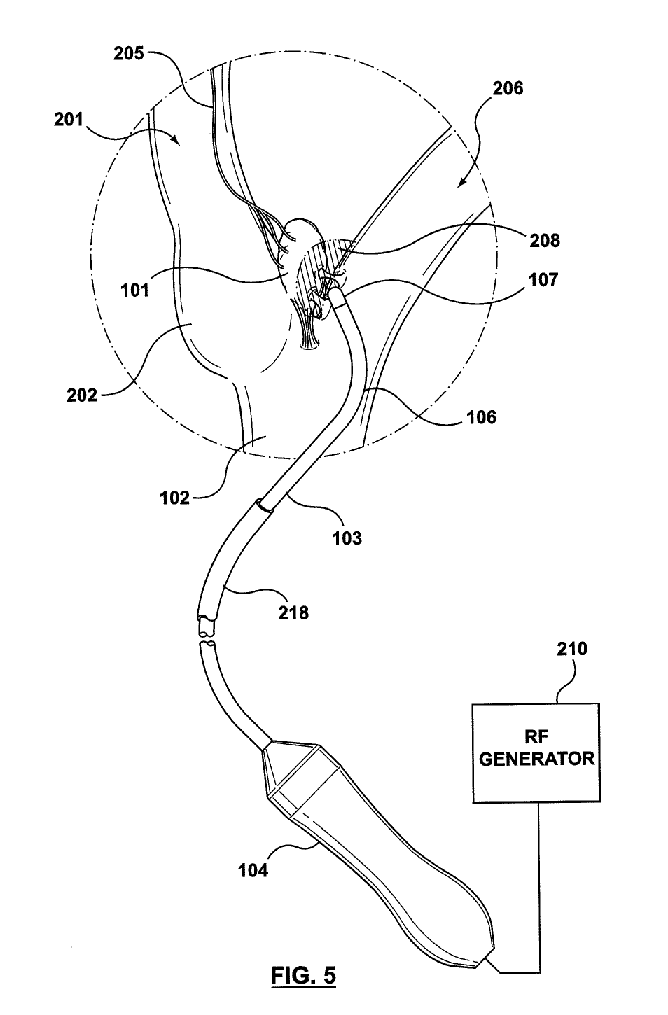

An embodiment of the present disclosure is shown in FIG. 5. A patient who is suffering from a cardiac, metabolic or pulmonary disease involving heightened SNS (e.g. dyspnea, hypertension, COPD or CHF), may be treated with a carotid body ablation catheter 103. The carotid body ablation catheter 103 may be inserted through a patient's vasculature to a common carotid artery 102 and applied to ablate a carotid body 101 associated with the common carotid artery 102. A carotid body ablation catheter 103 may ablate a carotid body or associated tissues (e.g. nerve supply, blood supply) through transvascular access (e.g. accessing a target across the wall of a blood vessel). For example, ablative energy may be applied to or through a blood vessel wall to reach the target tissue, or an element in association with a carotid body ablation catheter may puncture through a vessel wall to reach the target tissue. Transvascular access may be applied from an external carotid artery 206, an internal carotid artery 201, a common carotid artery 102, a carotid bifurcation 202, or a combination thereof. The carotid body ablation catheter 103 may comprise an energy delivery element 107 located at the distal tip. The energy delivery element 107 at the distal tip of the catheter 103 is shown in contact with the EC 206 vessel wall proximate to carotid body 101 in the process of creating a lesion 208. Optionally, multiple small lesions may be created to reduce trauma from a single larger lesion. For example, carotid body location can be identified using a selected imaging modality (e.g. CT, sonography, MRI, fluoroscopy) and the endovascular area proximate to the carotid body may be treated with multiple ablations. Tissue contact is generally needed for energy delivery that is dependent on thermal or electrical conduction (e.g. radiofrequency, thermal conduction, and cryogenic ablation) but may be less necessary for energies such as focused ultrasound or microwave ablation. Different modalities of delivering thermal energy to ablate tissue have distinct advantages and disadvantages. The proximal end of the catheter 103 may have a handle 104, which remains outside of the body. The energy delivery element 107 of the carotid body ablation device 103 applies energy to the carotid body to, for example, ablate the carotid body. Alternatively, a carotid body ablation catheter 103 may deliver a substance to ablate the carotid body, such as an ablative chemical or deliver material to embolize the carotid body.

An endovascular catheter may be delivered into a patient's vasculature via common approaches including femoral, radial, brachial artery or vein access, or even via a cervical approach directly into a carotid artery. An endovascular procedure may involve the use of a guidewire, delivery sheath, guide catheter, or introducer.

There may be danger of creating a brain embolism while performing an endovascular procedure in a patient's carotid artery, for example, a thrombus may be created by delivering ablation energy such as on a radiofrequency electrode, a piece of atheromatous plaque may be dislodged by catheter movement, or a cryogenic catheter could release a piece of frozen blood. In one embodiment of the present disclosure, in addition to a carotid body ablation catheter, an endovascular catheter is used to place a brain embolism protection device in a patient's internal carotid artery during a carotid body ablation procedure. An embodiment of this disclosure comprises occluding a patient's internal carotid artery. Blood flowing from a common carotid artery 102 would not flow through a connecting internal carotid artery 201, which feeds the brain, but instead would flow through the external carotid artery, which feeds other structures of the head that are much more capable of safely receiving an embolism. For example, as shown in FIG. 6 a brain embolism protection device in the form of an inflatable balloon 521 is placed in an internal carotid artery 201. An expandable device such as this may also facilitate endovascular ablation of a carotid body from an external carotid artery by pushing the carotid body closer to the wall of the external carotid artery and thus a lesion formed through the vessel wall of the external carotid artery may ablate the carotid body more effectively. The balloon 521 may be made from a soft, stretchable, compliant balloon material such as silicone and may be inflated with a fluid (e.g. saline or contrast agent) through an inflation lumen 524. Inflation fluid may be injected into an inlet port 527 by a syringe or by a computer controlled pump system 526. The balloon 521 may be placed, using a delivery sheath 530, at an ostium of the internal carotid artery, in an internal carotid artery a short distance (e.g. up to about 3 cm) from its ostium, or in a carotid sinus as shown in FIG. 6.

Contrast solution may be injected into the common carotid artery 102, for example through the delivery sheath 530 to allow radiographic visualization of the common 102, internal 201 and external 206 carotid arteries, which may assist a physician to position the occlusion balloon 521 or confirm occlusion. An endovascular ablation catheter 103 may place an energy delivery element 107 proximate a carotid body, for example at an inner wall of a medial segment of a carotid bifurcation or of an external carotid artery. It is expected that blood flow would carry any debris into the external carotid artery where it is harmless. Occlusion of an internal carotid artery may be done for a period of time that allows an ablation procedure and that is safe for the brain (e.g. less than or equal to about 3 minutes, or between about 1 to 2 minutes). Optionally, an occlusion catheter 520 may comprise a blood flow lumen 523 providing fluid communication from a vessel space proximal to an occluding balloon 521 to a vessel space distal to the occluding balloon 521. The proximal opening of lumen 523 may be spaced a sufficient distance from the occluding balloon such that blood entering the lumen is upstream from an ablation and clean of debris that could be caused by the ablation. The blood flow lumen 523 could optionally be used to deliver an occlusion catheter 520 over a guide wire (not shown), such as a rapid exchange guide wire or conventional guide wire. After the carotid body is ablated the brain embolism protection device may be deployed and removed from the patient or positioned on the patient's contralateral side in the event of ablating the contralateral carotid body.

When used in conjunction with a carotid body ablation catheter 103 that delivers a heat generating energy such as radiofrequency energy, microwave or ultrasound, an occlusive brain embolism protection device, as shown in FIG. 6 may provide additional benefit of increasing blood flow over an energy delivery element 107 and the vessel wall in contact with it. Increased blood flow may increase convective removal of heat from the energy delivery element and vessel wall allowing more energy to be delivered (e.g. greater power) to create a deeper ablation without adversely overheating the tissue or energy delivery element and avoiding unwanted tissue desiccation, tissue impedance rise, or thrombus formation.

It may be desirable to preserve a patent's baroreceptor located at the patient's internal carotid artery during a carotid body ablation procedure. An occlusion balloon may optionally be configured to cool surrounding tissue and provide thermal protection to the surrounding tissue during a thermal ablation procedure (e.g. RF, microwave, ultrasound ablation). As shown in FIG. 6, balloon 521 is inflated with a circulating fluid. Alternatively, an occluding balloon may be cooled by a non-circulating chilled fluid or be cooled by an endothermic phase change of a fluid such as Nitrous Oxide N₂O. Tissue surrounding the thermal protection, occluding balloon may be cooled to a temperature insufficient to cause cryogenic injury but sufficient to maintain a non-ablative temperature when subjected to ablative energy of a carotid body ablation catheter. For example, tissue temperature may be maintained at a temperature in a range below about 42° C. and above about −20° C. (e.g. in a range of about 5° C. to 37° C. or in a range of about 25° C. to 35° C.).

In another embodiment a brain embolism protection device may be a blood-permeable filter deployed in a patient's internal carotid artery. A filter may be a fine mesh or net connected to a deployable frame that expands to envelop a cross-section of an internal carotid artery distal to a bifurcation. Other embodiments of a blood-permeable filter may include wire-type expandable devices such as baskets or umbrellas. Such a filter may allow antegrade blood flow to continue to the brain while trapping and retrieving debris in the blood, preventing a brain embolism. Such a device may be deployed in an internal carotid artery prior to the placement of ablation catheter and retrieved following ablation.

An energy field generator 210 may be located external to the patient. Various types of energy generators or supplies, such as electrical frequency generators, ultrasonic generators, and heating or cryogenic fluid supplies, may be used to provide energy to the energy delivery element at the distal tip of the catheter. An electrode or other energy applicator at the distal tip of the catheter should conform to the type of energy generator coupled to the catheter. The generator may include computer controls to automatically or manually adjust frequency and strength of the energy applied to the catheter, timing and period during which energy is applied, and safety limits to the application of energy. It should be understood that embodiments of energy delivery electrodes described hereinafter may be electrically connected to the generator even though the generator is not explicitly shown or described with each embodiment.

An ablated tissue lesion at or near the carotid body may be created by the application of thermal energy from the energy delivery element 107 proximate to the distal end of the carotid body ablation device 103. The ablated tissue lesion may disable the carotid body 101 or may suppress the activity of the carotid body. The disabling or suppression of the carotid body reduces the responsiveness of the chemosensitive cells to changes of blood gas composition and effectively reduces the chemoreflex gain of the patient 100.

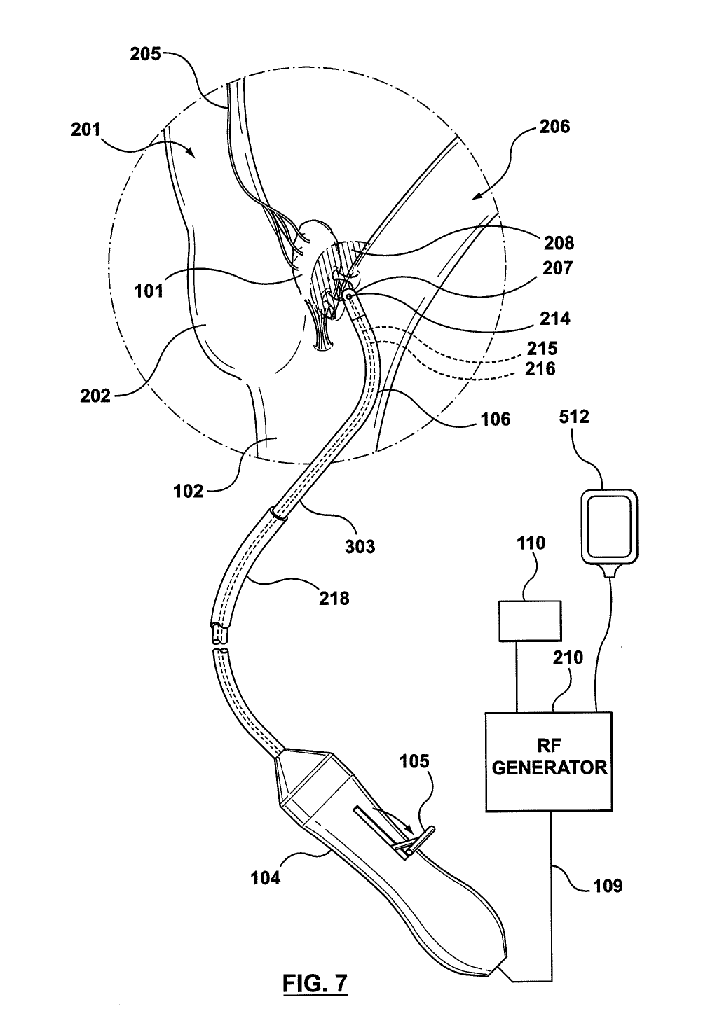

A carotid body ablation catheter may be configured to deliver radiofrequency electrical current (RF) and as such, the energy delivery element 107 may be a radiofrequency electrode 207, as shown in FIG. 7. RF is a rapidly alternating current that ablates tissue by generating heat in the tissue through ionic agitation, which is typically proportional to current density. Other factors that influence temperature generated in tissue include heat sinks (e.g. thermal convection due to blood flow) and tissue impedance. The volume of heated tissue is dependent on factors such as electrode size, RF power, duration of RF delivery, and waveform characteristics such as pulsing. In the embodiment shown in FIG. 7, the carotid body ablation catheter 303 is connected by wires 109 to an RF energy generator 210. The generator 210 may be included with a computer controller 110 that controls the application of energy to the electrode 207.

In this illustration a reference electrode 212 is placed on the surface of the body of the patient 100 such as on the skin of the chest or the back of the patient. The reference electrode 212 establishes a current return path to the RF generator 210 for current flowing from the electrode 207, through the body of the patient and to the reference electrode 212. The arrangement in which current flows through a reference electrode 212 and an active electrode 207 is generally referred to as a monopolar arrangement. The reference electrode 212 has a relatively large surface area to minimize current density and avoid skin burns.

A RF electrode 207 may be made from an electrically conductive material (e.g. stainless steel, gold, platinum-iridium) and may be less than or equal to about 3 mm in diameter (e.g. between 1 to 2.5 mm) and less than or equal to about 5 mm in length (e.g. between 1 and 4 mm). A temperature sensor 214 (e.g. thermocouple, thermistor, fluoroptic thermometry sensor) may be located in, near, or at the surface of the RF electrode 207. In FIG. 7 a thermocouple 214 is located in the RF electrode 207 and is connected to two conductors 215 and 216. The conductors travel through the catheter body (e.g. through a lumen) from the distal to proximal end and are connected to wires 109 allowing the thermocouple to communicate with the RF generator 210. Conductors 215 and 216 may be, for example a copper and constantan conductor, respectively, such that joining the conductors 215 and 216 via solder, laser welding or the like creates a thermocouple junction. Copper conductor 215 may be used to carry both a thermocouple signal and deliver RF energy to the electrode 207 as shown in FIG. 7. Alternatively, a separate conductor (not shown) may deliver RF energy to the electrode. The catheter 303 may further comprise deflectable section 106 near the distal end, for example within about 3 cm from the distal end of the catheter 303. Deflectable section 106 may be controllably deflected by a physician by initiating an actuator 105 integrated in to a handle 104. For example, the actuator may be connected to a control wire (not shown) that travels the length of the catheter (e.g. through a lumen) to the deflectable section 106. The control wire may be connected to a structure that is biased to compression or tension of the control wire such that the structure deforms in its biased configuration. Other alternative embodiments for controlling deformation of the deflectable section may be used for example, electrically or thermally activating a shape memory alloy structure, or electrically activating an electroactive polymer structure.

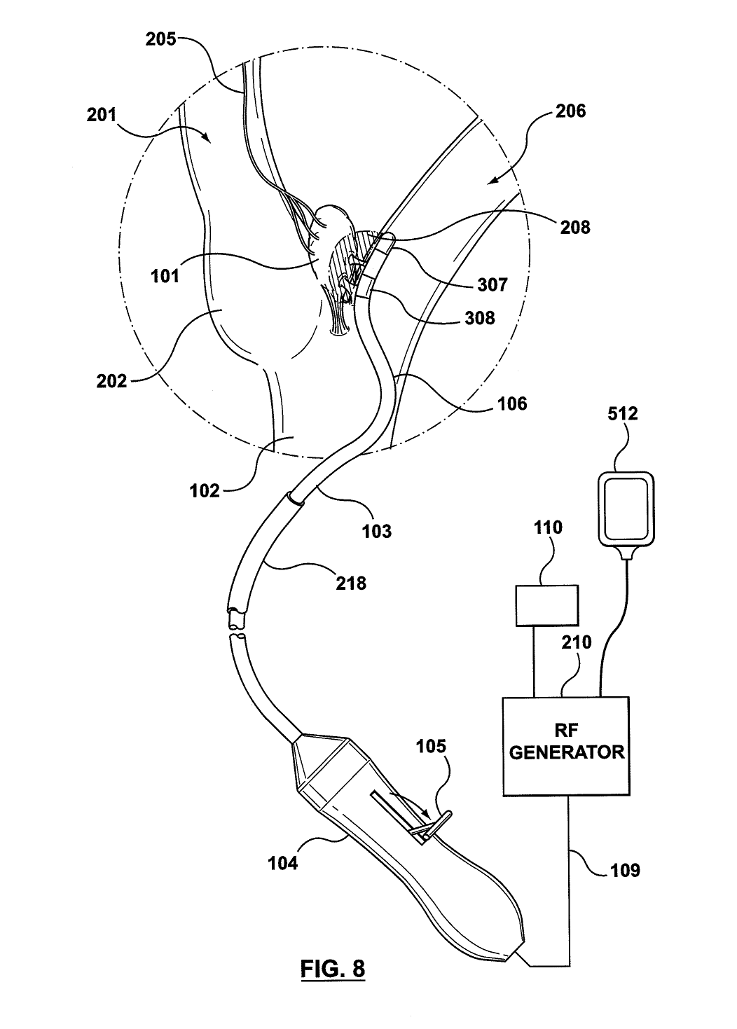

Alternatively, as shown in FIG. 8, multiple electrodes may be arranged at or near the distal tip region of a carotid body ablation catheter 103 such that current flows from an active electrode 307 to a return electrode 308 to create an energy field, (e.g. an electric field) in the region adjacent the electrodes 307 and 308 that ablates tissue. Such an arrangement is generally referred to as a bipolar configuration. Active and return electrodes may be located on the same shaft of a catheter as shown in FIG. 8. For example, the electrodes may be about the same size and shape and be distanced between about 0.5 mm and 10 mm (e.g. between about 1 and 4 mm) apart from one another. Alternatively, the electrodes may be different sizes so current density is greater around the smaller electrode creating a greater thermal effect. Another embodiment of a bipolar arrangement involves having an active electrode and a return electrode placed on separate shafts (not shown).

Bipolar and monopolar electrosurgery techniques for general use and for cardiac ablation are well known in the field of catheters for RF ablation of tissue. Bipolar and monopolar catheters for RF ablation adapted for placement, fixation upon and ablation of carotid septum are novel and serve specific purpose of protecting vulnerable structures proximate to the carotid septum from unintended damage from heating and irreversible damage by RF energy.

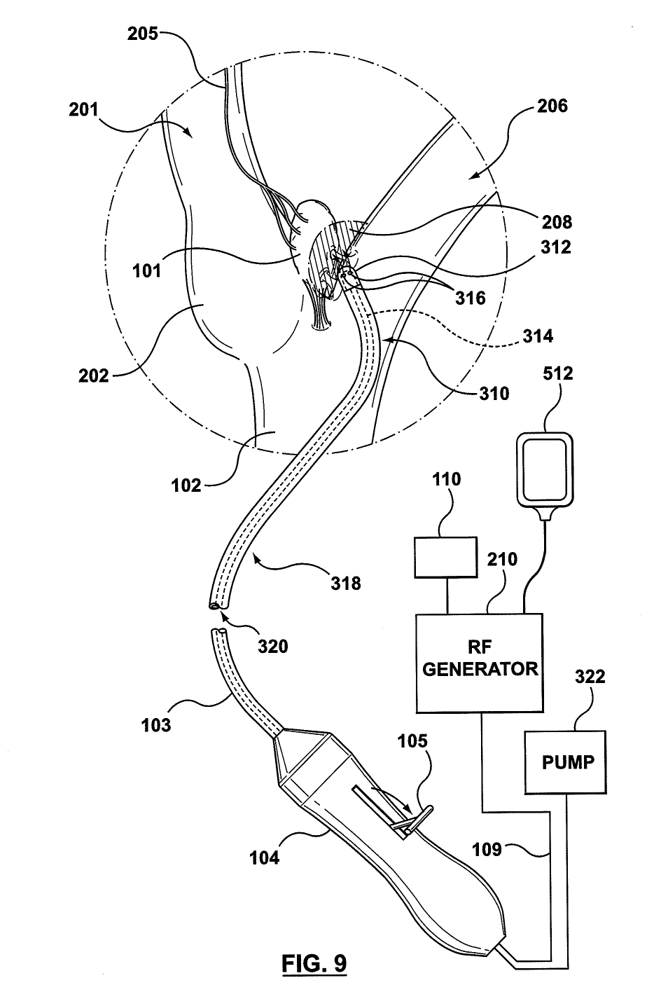

As shown in FIG. 9, a RF ablation catheter may additionally be configured to provide cooled RF energy delivery. For example, a catheter 318 may contain a lumen 314 in fluid communication with one or more RF electrodes 312 to irrigate a cooling fluid 320 (e.g. room temperature or chilled saline) to the RF electrode. The cooling fluid may exit the RF electrode through irrigation ports 316 and enter the blood stream. Alternatively, cooling fluid may be circulated through a cavity or lumen in a cooled RF electrode and then circulate back through a lumen in the catheter shaft to be deposited elsewhere in the patient's vasculature or outside the body. A cooled RF system may additionally comprise a cooling fluid source and pump 322. The benefit of cooling a RF electrode may be reduction of the risk of heating blood, which may create a clot or emboli. Furthermore, cooled RF may produce ablations deeper in the tissue or may heat the surface layers of the tissue less. These benefits may be particularly advantageous in a carotid vasculature since the internal carotid feeds the brain and the targeted tissue may be beyond the vessel's surface (e.g. 2 to 5 mm deep from the inner surface of a vessel wall).

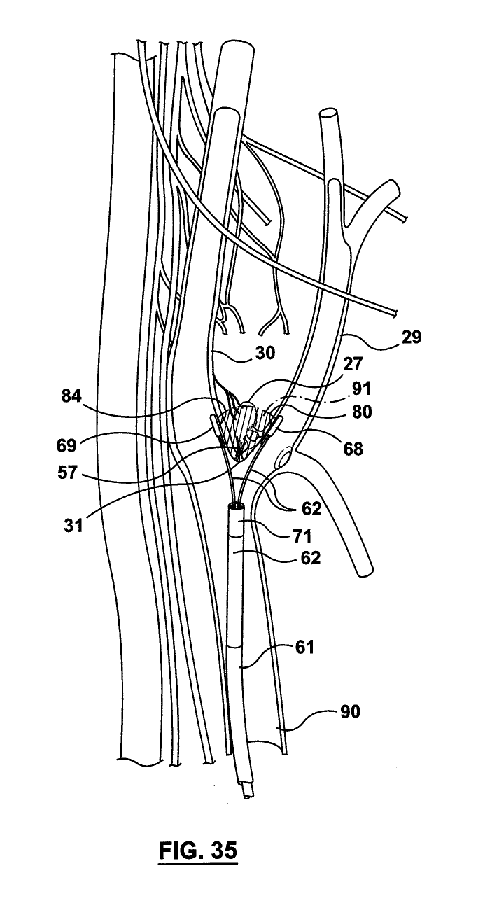

FIG. 35 illustrates a distal section of a carotid body ablation catheter configured to couple with the form of a carotid bifurcation or carotid septum wherein said coupling facilitates placement of ablation elements in an ideal positioning range for safe and effective carotid body modulation. Carotid septum coupling may be achieved, for example by a catheter having two arms such that one arm is placed on one side of a septum in an external carotid artery and the other arm is placed on an opposing side of the septum in an internal carotid artery. The catheter may comprise a means to open and close the arms to facilitate placement of the arms on both sided of a septum and application of electrode contact force with tissue. A means to open and close the arms may be comprise an active means, for example user controlled actuation of a pull wire that opens or closes the arms. Alternatively, a means to open and close the arms may comprise a passive means, for example, preformed elastic members that apply a closing force. A means to open and close the arms may comprise a combination of active and passive means.

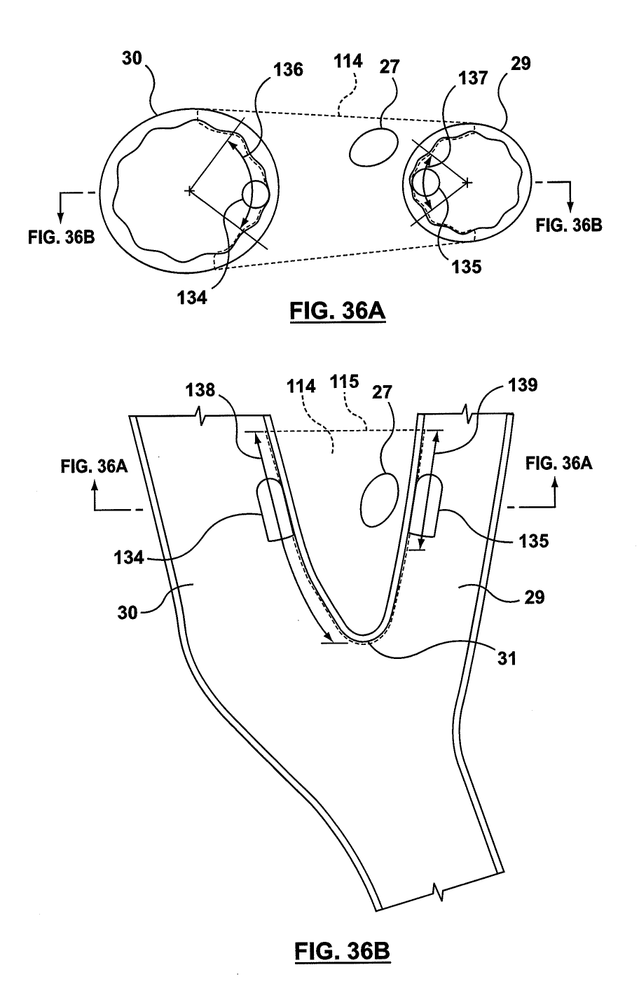

FIGS. 36A and 36B illustrate an example of ablation element positioning that may effectively and safely ablate a carotid body 27. FIG. 36A shows, outlined with a dashed line, a transverse cross-section of an intercarotid septum 114 bordered by an internal carotid artery 30 and an external carotid artery 29. In this embodiment, a first ablation element 134 is placed in the internal carotid artery 30 in contact with the vessel wall within a vessel wall arc 136 directed toward the external carotid artery; a second ablation element 135 is placed in the external carotid artery 29 in contact with the vessel wall within a vessel wall arc 137 directed toward the internal carotid artery. Each vessel wall arc 136 and 137 is contained within limits of the intercarotid septum 114 and comprises an arc length no greater than about 25% (e.g. about 15 to 25%) of the circumference of the respective vessel. In this example, the ablation elements 134 and 135 may be bipolar radiofrequency electrodes or irreversible electroporation electrodes wherein electrical current is passed from one electrode to the other electrode through the intercarotid septum. Placement of ablation elements as described may facilitate targeted deposition of energy and the creation of an ablation lesion that is contained within the intercarotid septum, thus avoiding injury of non-target nerves that reside outside the septum, and an ablation that is sufficiently large (e.g. extending approximately from the internal carotid artery to the external carotid artery) to effectively modulate a carotid body or its associated nerves. Specifically this configuration facilitates deposition of energy along the line between the electrodes and inhibits it in the medial direction (towards the spine).

FIG. 36B shows, outlined with a dashed line, a longitudinal cross-section of an intercarotid septum 114 bordered by an internal carotid artery 30, an external carotid artery 29, a saddle of a carotid bifurcation 31 and a cranial (towards the head) boundary 115 that is between about 10 to 15 mm cranial from the saddle 31. In this example, the first ablation element 134 is placed in the internal carotid artery 30 in contact with the vessel wall within a first range 138; a second ablation element 135 is placed in the external carotid artery 29 in contact with the vessel wall within a second range 139. The first range 138 may extend from the inferior apex of the bifurcation saddle 31 to the cranial boundary 115 of the septum (e.g. about 10 to 15 mm from the bifurcation saddle). The second range 139 may extend from a position about 4 mm superior from the bifurcation saddle 31 to the cranial boundary 115 of the septum (e.g. about 10 or 15 mm from the bifurcation saddle). As an example, an ETAP catheter may be configured to place a distal tip of a 4 mm long electrode in an internal carotid artery about 10 mm from a carotid bifurcation and a distal tip of a second 4 mm long electrode in a corresponding external carotid artery at about 10 mm from the carotid bifurcation. The electrodes 134 and 135 may be equidistant from the saddle 31 or they may be unequal distances from the saddle.

The catheter shown in FIG. 35 is equipped with energy delivery elements (e.g. RF electrodes) placed on two sides of the carotid septum with an intention of delivering and containing deposition of energy within the septum. Energy delivery elements may be communicating elements of a bipolar circuit, such as a bipolar radiofrequency configuration, in which ablative energy (e.g. radiofrequency electrical current) is passed from one energy delivery element through a carotid septum to the other energy delivery element. A bipolar configuration may create an ablation that is large enough to substantially affect contents of a carotid septum and sufficiently ablate a target (e.g. carotid body, nerves associated with a carotid body), while containing the ablation within the carotid septum to safely avoid injury to non-target nerves and organs.

In an alternative embodiment, bifurcation coupling may be used to position an ablation element, such as a radiofrequency electrode, in an external carotid artery in contact with a carotid septum and directing ablation energy toward a dispersive electrode placed in an internal carotid artery that is not necessarily in contact with the septum.

In an alternative embodiment, bifurcation coupling may be used to facilitate positioning an ablation element in an external carotid artery and aiming energy delivery at the carotid septum. For example, a first arm comprising an ablation element may be placed in an external carotid artery while a second arm is placed in an internal carotid artery and does not comprise an ablation element but is used to position the ablation element in the external carotid artery at a suitable distance from a carotid bifurcation 139 of FIG. 36B and provide rotational alignment such that energy is delivered in a target range 137 of FIG. 36A. Ablation energy may be for example, monopolar radiofrequency, cryogenic energy, ultrasound, laser, or microwave.

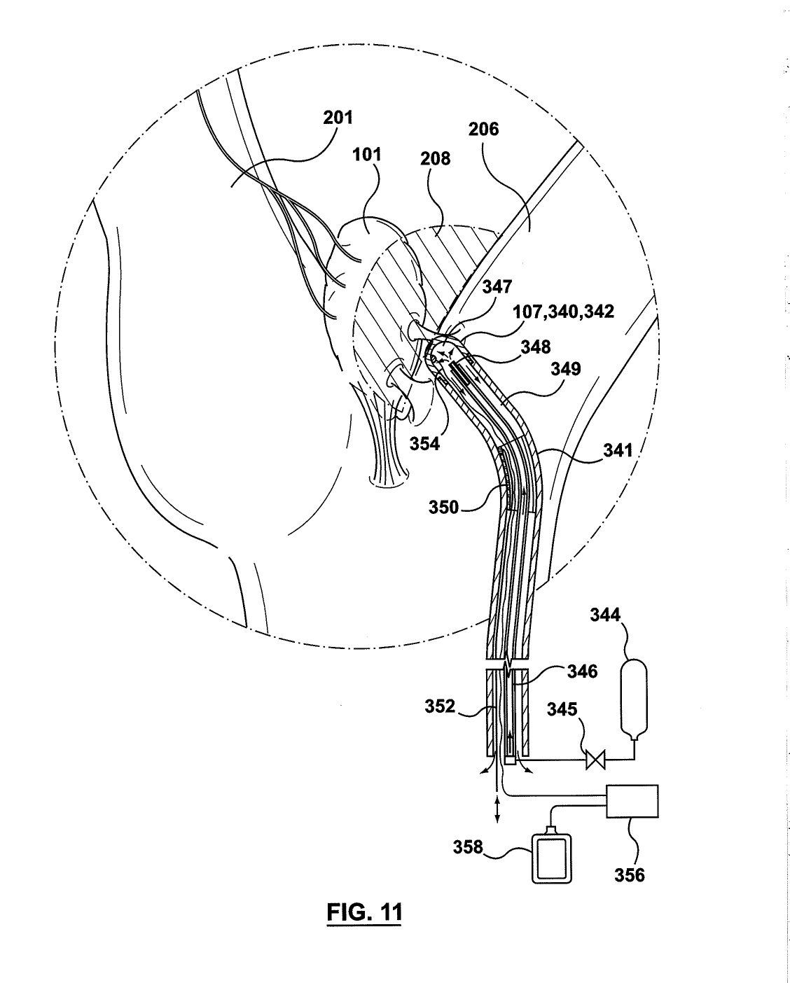

A carotid body ablation catheter may be configured to cryogenically ablate tissue and as such, the energy delivery element 107 may be a cryogenic applicator 340, as shown in FIG. 11. Cryogenic ablation may possess benefits for a carotid body ablation procedure. For example, a non-ablative cold temperature (e.g. −15 to 5° C.) may be induced in a carotid body to temporarily block activity, which may be used to test if a permanent carotid body ablation would have desirable effects such as decreasing chemosensitivity. Other benefits may include less pain or less risk of vessel stenosis compared to high temperature ablation, a deep ablation, and cryo-adhesion, wherein a cryogenic applicator may stick to a vessel wall when cold, which could provide excellent contact stability. Another benefit is that the formation of ice in tissue may be viewed with ultrasound or magnetic resonance imaging to confirm an effective ablation zone and avoid ablation of nearby structures such as the carotid baroreceptors. FIG. 11 is a schematic illustration of a point-ablate cryo-catheter 341 having a cryogenic applicator 340 configured as a metal tip 342. A source of cryogen 344 (e.g. a pressurized canister of nitrous oxide N₂O) is in fluid communication with a supply tube 346 and a valve 345 controls flow. Cryogen in substantially liquid form flows through the supply tube 346 to a distal end of the cryo catheter 341 and through a restriction orifice 348 before exiting into an expansion chamber 347 defined by a cavity in the metal tip 342. The restriction orifice 348 may restrict flow of the cryogen, influencing a pressure differential between the supply tube and the expansion chamber. A large drop in pressure as the cryogen enters the expansion chamber 347 allows the cryogen to change phase from substantially liquid to substantially gas, which is an endothermic reaction absorbing a large amount of heat from surrounding structures. Heat is pulled from tissue through the metal tip 342 and the tissue is cryo-ablated. Control of cryogen flow rate by the valve 345 may control degree of cooling allowing a temporary block and permanent cryo-ablation to be performed with the same device. Gaseous cryogen flows from the expansion chamber 347 through an exhaust lumen 349 and may be exhausted to atmosphere. A point-ablate cryo-catheter 341 may be configured for controlled deflection to allow a physician to deflect or bend a distal region of the cryo-catheter 341 placing the cryo-applicator 342 in contact with a target site. For example, the catheter may comprise a compression-biased structure that bends in a predefined direction upon application of force. For example, a pull wire 352 pulled by an actuator on a handle (not shown) may be connected to a distal end of a laser cut metal tube 350, creating compression of the metal tube causing it to bend in a biased direction. Optionally, the metal tip 342 may be electrically connected to a conductor 354, which may provide electrical communication with an electrical stimulation signal generator 356. A return electrode 358 connected to the electrical stimulation signal generator 356 may be placed on the patient's skin to complete the circuit. This configuration may allow an electrical stimulation signal to be applied to the target site to confirm proximity to a carotid body or safe distance from a baroreceptor (e.g. by producing a physiologic response to the stimulation such as changes in blood pressure, heart rate, ventilation). Other embodiments of cryogenic catheters may be used to ablate a carotid body such as cryo-balloon applicators.

An endovascular catheter may alternatively be designed and used to deliver an ultrasound transducer. Intravascular Ultrasound (IVUS) is a medical imaging technology in which a small ultrasound transducer is mounted to a catheter and delivered through a patient's vasculature to provide an ultrasound image from inside the vessel out through blood and into layers of the vessel wall and a short distance beyond. IVUS was developed initially for intravascular imaging of coronary arteries to investigate vascular structures. An IVUS catheter may be applied both extravascularly or intravascularly to the cervical carotid arteries to obtain ultrasound images of carotid arteries and surrounding tissues. IVUS may be a useful tool to locate and visualize a carotid body so an ablation device can be directed to a target site. IVUS may also be useful in imaging a diseased carotid artery bifurcation in order to image plaque that can suggest best approaches to the ablation procedure.

An IVUS catheter can be for example an intravascular ultrasound imaging device Eagle Eye Gold catheter (0.014 guidewire compatible, 2.9 Fr outer shaft diameter) by Volcano Corp, Rancho Cordova, Calif.

Data obtained from IVUS may be processed by a computer algorithm to produce useful images, for example to assess material properties, distinguish between plaque and vessel tissue, or identify blood flow. For example, IVUS Virtual Histology™ (VH-IVUS) may be used to evaluate the pathological properties of plaque contained within a carotid artery and healthy tissues as well as ablation induced scar tissues. VH-IVUS displays plaque composition within four color categories (e.g. fibrous, fibro-fatty, calcification and necrotic core) being able to offer detailed tissue characterization of soft to hard plaque components.

It is anticipated that improvements can be made to IVUS to make it more usable for carotid artery procedure and CB imaging. A brain embolism protection device may be used in conjunction with IVUS to catch plaque loosened from an artery wall. A catheter-based ultrasound transducer could also be configured to focus ultrasound waves on a target tissue a short depth from a vessel wall to ablate a carotid body.

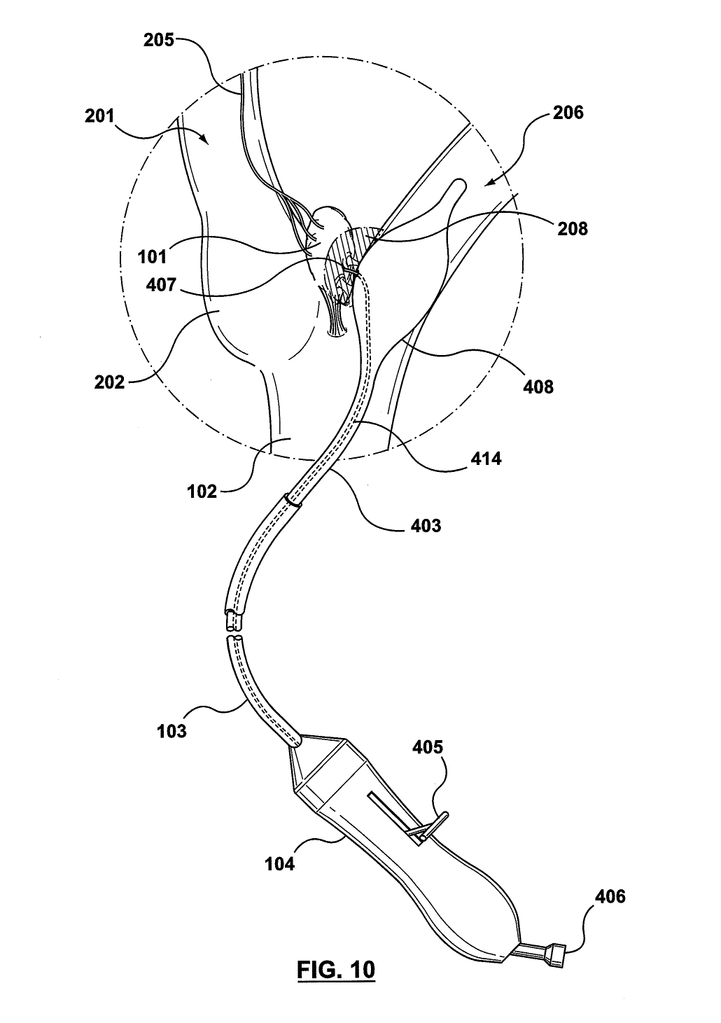

An endovascular catheter may alternatively be designed and used to deliver an agent across a vessel wall to affect target tissue. As shown in FIG. 10, a transvascular injection catheter 403 has a deployable micro-needle 407 having a lumen 414 in fluid communication through the catheter shaft to an injection hub 406. Once the micro-needle is deployed through a vessel wall (e.g. the wall of an external carotid) a contrast solution may be injected to verify position in a target tissue. An ablative agent, sclerosing agent or a neural disruptive agent may be injected into a target tissue. An example of an agent that may be used to disable sympathetic signaling from a carotid body is Guanethidine, which is known to cause sympathectomy, by inhibiting mitochondrial respiration, and induce an immune response. A temporary neural blockade (e.g. Bupivicane) may be injected to test a response to therapy prior to a more permanent ablative or disruptive injection. Following injection, the micro-needle may be retracted and the catheter removed from the patient. Deployment and retraction of the micro-needle may be achieved with an actuator 405 on a catheter handle. For example, an actuator 405 may be connected to a control wire running through a catheter shaft to a deployable structure 408 at the distal end of the catheter. The deployable structure 408 may be, for example, a deployable mesh, cage, basket, or helix that radially expands to secure the distal end of the catheter in the vessel and causes the micro-needle to protrude. The deployable structure may be an inflatable balloon that is deployed by injecting air or liquid (e.g. saline) into a hub in a handle. Alternatively, a micro-needle may be deployed by a separate control or actuator that advances the micro-needle out of the catheter.

Another example of a transvascular approach is to use an endovascular catheter designed with a deployable micro-needle having an energy delivery element that may be deployed through a vessel wall (e.g. a wall of an external carotid artery) and placed in or proximate to target tissue. The energy delivery element may be, for example, a cryo, RF or electroporation electrode.

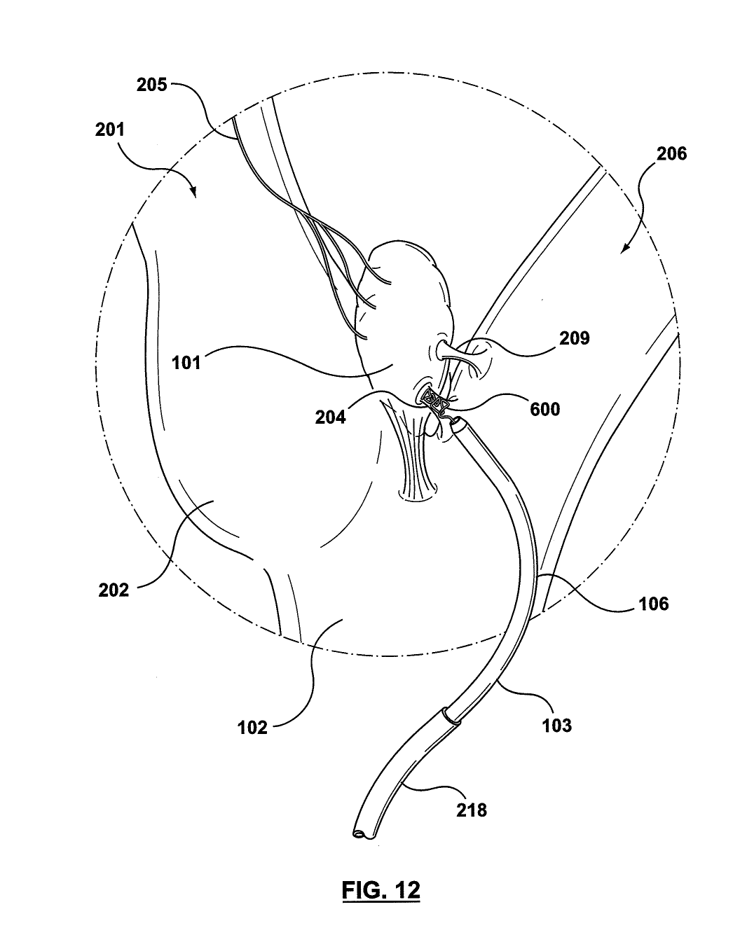

Another embodiment of the present disclosure, as shown in FIG. 12 involves embolizing the carotid body 101 by blocking blood flow to the carotid body. For example, a microcatheter may be inserted into small arteries 209 or 204 that feed a carotid body and an implantable occlusion device 600 can be placed in the artery. The implantable occlusion device 600 may be microspheres, filaments, a wire coil, an injectable foam, cement, or hardening composition. Examples of injectable microspheres include 500 to 700 micron spherical polyvinyl alcohol particles (SPVA) such as Contour SE Microspheres made by Boston Scientific, which are commonly used to infarct fibroids. Alternatively 700 to 900 μg or even 900 to 1200 μg particles may be used.

An alternative method of embolizing the carotid body may be to ablate or cause stenosis of the blood vessels feeding the carotid body, for example using an endovascular ablation catheter such as a RF catheter to apply thermal energy to the blood vessels 209. This may be accomplished by applying ablative energy (e.g. RF) from a vessel surface such as an external carotid artery. Alternatively, a small catheter may be inserted into a vessel that feeds or drains a carotid body and ablative energy may be applied directly to the walls of these vessels to cause them to close.

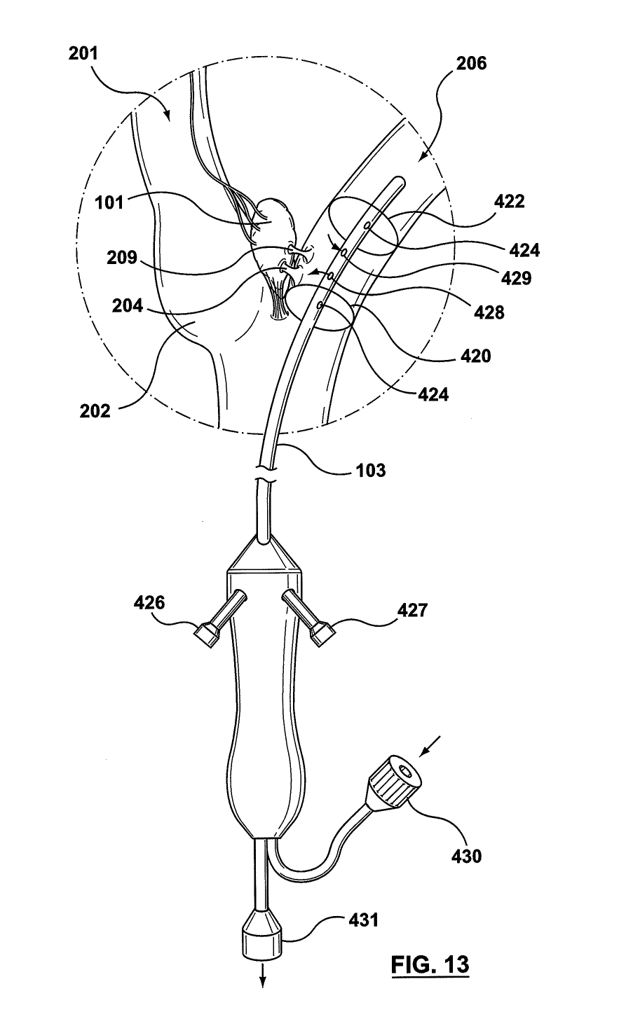

Another alternative embodiment of an embolization device and method, as shown in FIG. 13, involves a catheter 103 having two inflatable occluding balloons, one proximal 420 and one distal 422. Each of the occlusion balloons may be inflated or expanded by injecting gas or fluid through balloon inflation ports 424 that may be in fluid communication via a lumen through the catheter to one or two inflation hubs 426 and 427 on the proximal end of the catheter for control of inflation of each balloon either independently or concurrently. The catheter would be placed such that the proximal and distal occluding balloons 420 and 422 would occlude the EC 206 proximal and distal to small arteries 204 and 209 feeding a carotid body 101. The catheter may further comprise an injection port 428 and an evacuation port 429 both positioned on the catheter shaft between the proximal and distal occlusion balloons. The injection and evacuation ports are in fluid communication via lumens (not shown) in the catheter shaft to injection 430 and evacuation 431 hubs, respectively. The occlusion balloons create an isolated segment of the vessel (e.g. EC) that is in fluid communication with small vessels connected to a carotid body, and also with lumens traveling through the catheter to and from evacuation 431 and injection 430 hubs. To embolize small vessels feeding a carotid body, an embolization agent (such as those previously described) may be injected through the injection hub 430 which will travel through the catheter and exit the injection port into the isolated segment in the EC and into the feeding vessels 204 and 209. To relieve fluid pressure, blood or injected agent may be evacuated from the isolated segment of the vessel through evacuation port 429 and out of the catheter via the evacuation hub 431. Evacuated fluid may be released to atmospheric pressure or may be pulled out with negative pressure (e.g. using a syringe or vacuum). By occluding an isolated segment in the EC around the vessels that feed the carotid body, injected fluid may be controlled and removed so it does not perfuse downstream through the EC or perfuse into the IC.

Prior to embolization, the same device illustrated in FIG. 13 may be used to visualize the carotid body on fluoroscopy and confirm correct placement and occlusion of the isolated segment of the vessel. A contrast solution may be injected through the injection hub 430 which will travel through the catheter and exit the injection port in to the isolated space in the vessel and into the carotid body 101.

Additionally or alternatively, a contrast solution may be injected into the isolated space following the removal of the embolizing agent to confirm that the carotid body has been embolized.

A device similar to the embodiment illustrated in FIG. 13 may be used in an alternative method to ablate a carotid body using chemical ablation. In this method, a chemical agent is delivered to the isolated segment of the vessel such that it is perfused through vessels feeding the carotid body and into the carotid body. The chemical agent may be, for example, an ablative or neural blocking agent. By occluding an isolated segment in the EC around the vessels that feed the carotid body, injected fluid may be controlled and removed so it does not perfuse downstream through the EC. The isolated segment of the vessel may be flushed by injecting a flushing liquid (e.g. saline) into the isolated segment via the injection port 428 and removing the fluid in the isolated segment via the evacuation port 429.

The same device may be used, prior to chemical ablation, to inject a contrast solution that may be viewed using fluoroscopy to ensure the vessel (e.g. EC) is properly occluded and the small vessels 204 and 209 feeding the carotid body 101 are targeted.

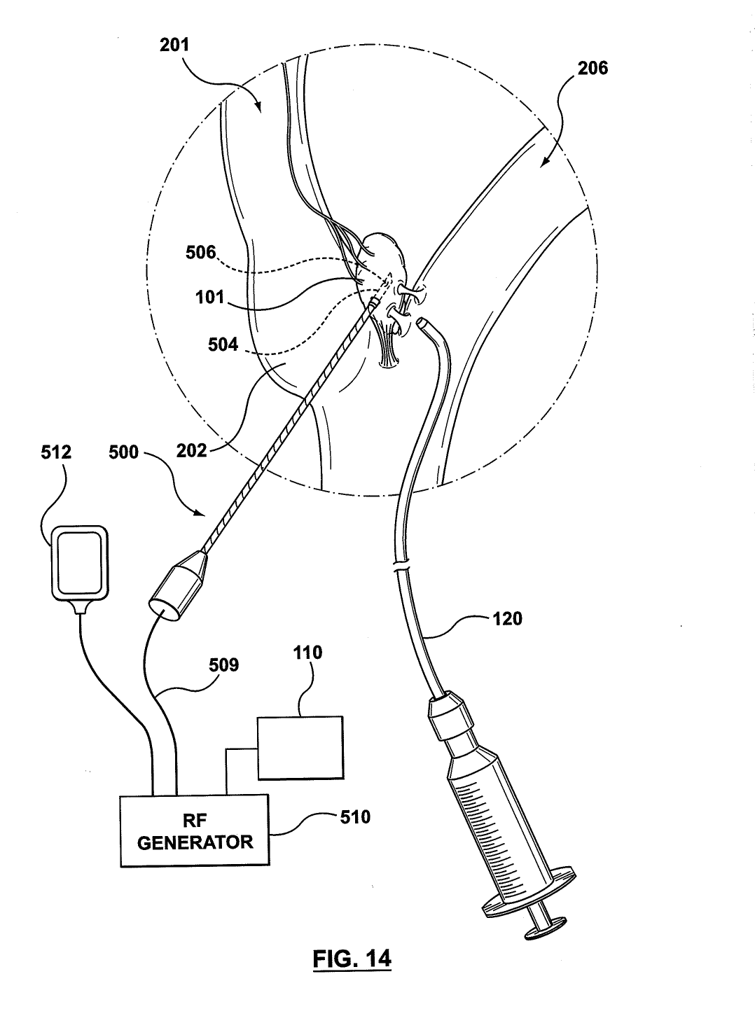

Another embodiment of the present disclosure, as shown in FIG. 14 involves providing visualization (e.g. radiographic visualization, Computer Tomography (CT), MRI, or ultrasound) of a carotid body and insertion of a percutaneous ablation device (e.g. a radiofrequency ablation needle 500, a chemical/drug delivery needle, a cryo-probe, a cryo-needle). Visualization of the carotid body or carotid arteries may facilitate safe and effective insertion of a percutaneous ablation device. Several embodiments of visualization methods and devices as well as percutaneous ablation devices are described hereinafter.

In FIG. 14, the visualization technique shown involves an injection catheter 120 that is used to inject radiographic contrast solution. Contrast may be injected, as shown, in close proximity to arterioles feeding a carotid body so the contrast enters the carotid body allowing it to be seen on a radiograph (e.g. fluoroscope). Alternatively, the contrast may be injected upstream of a carotid body such as in a common carotid artery, to allow visualization of the carotid bifurcation and the internal and external carotid arteries. Even if the carotid body itself is not clearly seen the carotid bifurcation or carotid arteries may be used as landmarks and structures to avoid.

Additionally or optionally, endovascular devices visible with an imaging modality may be placed in the internal jugular vein. It may be desired to avoid damage to the jugular vein while ablating the carotid body and carotid body nerves. Since the jugular vein and common carotid artery are very close it is beneficial to improve visualization, stability and location of both.

Alternatively, a distal region of an endovascular catheter may be placed in a patient's vasculature proximal to a targeted carotid body to provide assistance during a percutaneous carotid body ablation procedure. Such a catheter may assist a percutaneous ablation procedure by providing additional stability, a fiduciary marker, or thermal protection.

Increased stability could be beneficial to hold the vessel(s) in place while skin is punctured and a percutaneous ablation device is advanced to a target. Increased stability may be provided, for example, by a structure that increases stiffness such as an expanded balloon, mesh or cage.

A fiduciary marker may be, for example, a guidewire, a radiopaque mesh or cage or balloon that allows a vessel proximate to a targeted carotid body to be visualized radiographically and used as a landmark while placing the percutaneous ablation device in a location where a carotid body is expected such as at a lateral side of a carotid bifurcation. Visualizing a proximate vessel may also increase safety by helping a physician avoid puncturing the vessel or ablating too close to the vessel wall.

Thermal protection may benefit a percutaneous ablation procedure that thermally ablates a carotid body (e.g. RF, cryo, direct heat) by maintaining a non-ablative temperature in the vessel wall. For instance, if a RF probe is inserted near or in to a carotid body and a thermal lesion is created to ablate the carotid body, the lesion may expand beyond the carotid body and a carotid artery wall that is close to the RF probe may be in danger of being injured, especially if the RF electrode is very close or touching the carotid artery wall. A thermal protection catheter placed within the artery may actively cool the vessel wall and maintain a non-ablative temperature. Additionally, when a thermally protective element is placed in a carotid sinus 202 it can also protect a carotid baroreceptor from injury. It is understood that a similar thermal protection catheter could be inserted in to the internal jugular vein proximate to the carotid artery to avoid unintentional thermal damage intended to ablate a carotid body.

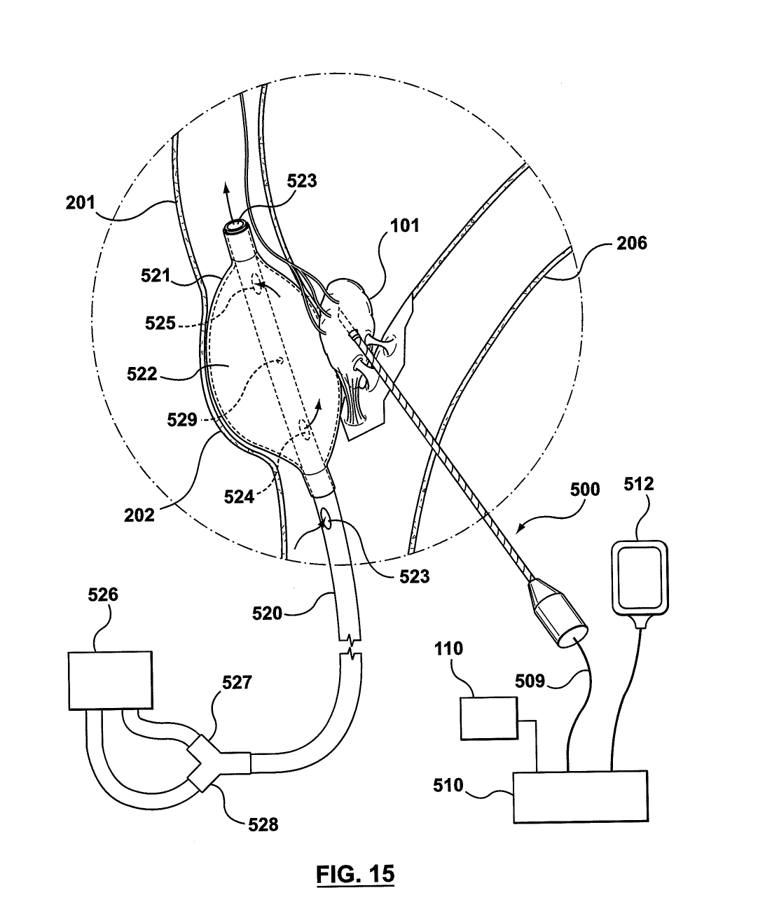

For example, as shown in FIG. 15, a catheter 520 used to assist a percutaneous ablation procedure comprises an inflatable balloon 521 that is positioned with visual guidance (e.g. fluoroscopy) in a vessel proximal to a targeted carotid body 101 such as an internal 201 or external 206 carotid artery or a carotid bifurcation. The balloon 521 may be made from compliant balloon material and inflated by injecting a contrast solution 522 into the balloon through an inflation lumen 524. Once placed, the balloon may safely occlude the vessel for a short time while a percutaneous ablation procedure is performed. Optionally, the catheter 520 may further comprise a lumen 523 in fluid communication with the proximal and distal sides of the balloon 521 to allow blood flow to continue to flow through the vessel. Optionally, the inflation fluid may comprise a contrast solution that is cooled or is replenished to maintain cool temperature. Inflation fluid may be exhausted through an exhaust lumen 525 while cooled inflation fluid is replenished through the inflation lumen to allow the pressure in the balloon 521 to be approximately maintained. Cooled contrast solution may be supplied and removed by a pump system 526 connected to inlet 527 and outlet 528 ports on a proximal region of the catheter 520. An optional sensor 529 (e.g. temperature sensor, pressure sensor) may be positioned inside the balloon 521 and connected to the pump system 526. A signal from the sensor 529 may be used in a feedback control algorithm to control flow of inflation fluid 522 or temperature in the balloon. A cooled balloon may be used to maintain a non-ablative temperature in the vessel wall while a proximal carotid body is being thermally ablated.

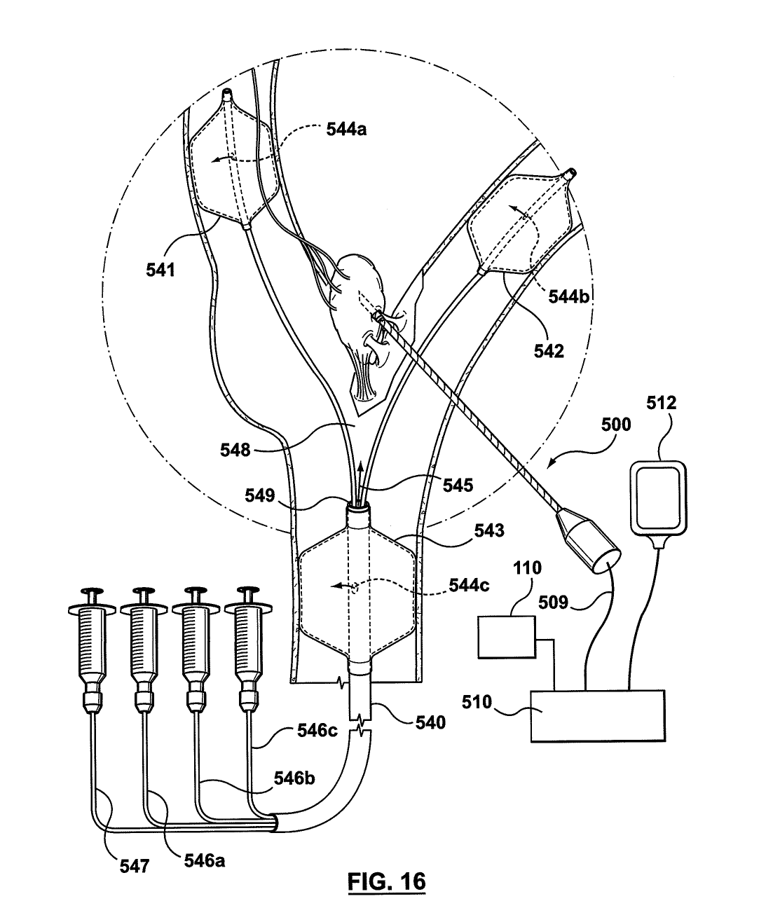

As shown in FIG. 16, an alternative embodiment of a catheter 540 used to assist a percutaneous ablation procedure comprises multiple inflatable balloons 541 and 542 that are placed to occlude an internal carotid artery 201 and an external carotid artery 206 distal to a carotid bifurcation 200 and carotid body 101 to allow contrast solution injected proximal to the occluding balloons to pool in the arteries, which further allows radiographic visualization while performing a percutaneous carotid body ablation procedure. An optional third balloon 543 may be used to occlude the common carotid artery. Each balloon may be inflated by injecting fluid through inflation lumens 544 in communication with injection ports 546 at the proximal region of the catheter(s). Contrast solution 545 may be injected into the occluded vessel space 548 via a lumen 549 in fluid communication with injection port 547. Optionally, the contrast solution 545 injected into the occluded vessel space 548 may be cooled to provide thermal protection as previously described. Once the occluded vessel space 548 is filled with contrast a percutaneous ablation device may be inserted under fluoroscopy or assisted by ultrasound (e.g. IVUS) to the target carotid body, or space relative to the occluded vessel space where a carotid body is expected to be. An electrical stimulation signal may be delivered to confirm proximity to a carotid body or avoidance of a baroreceptor. Ablation energy may then be delivered. Following ablation, the occluding balloons may be deflated by extracting inflation fluid from the balloons through the inflation lumens. The balloon in the external carotid artery may be deflated first followed by the balloon in the common carotid artery, allowing blood to flow through the external carotid artery before deflating the balloon in the internal carotid artery.

Ultrasound imaging (e.g. sonography) may be a useful technology to visualize a carotid body and surrounding structures (e.g. carotid arteries, jugular vein) to assist the insertion of a percutaneous ablation device or other percutaneous device that may be used in a carotid body ablation procedure (e.g. guide needle wire, micro-needle, needle, stimulation electrode probe). Ultrasound imaging subjects patients and physicians to less radiation and allows soft tissue to be visualized than compared to fluoroscopy and thus may be an alternative technique to contrast injection and radiography for localization of a target. As shown in FIG. 17 a transducer/transceiver may be placed on an external surface of a patient's skin and aimed toward the patient's targeted carotid body. Gel may be used to aid transducer contact and improve the ultrasound image. The transducer may be connected to an ultrasound system having a display screen. Blood filled vessels will appear hyperechoic and vessel walls will appear bright. Color flow Doppler may additionally be used to visualize vessels. A technique used to locate a carotid body using an ultrasound transducer may comprise placing a transducer 560 on the side of a patient's neck over the common carotid artery 102 to view the common carotid artery as a large round cross-section, which can be further illuminated with Doppler. The transducer 560 may then be slid in a cranial direction while observing the image of the common carotid artery cross-section. As the transducer approaches the carotid bifurcation 200 the image of the cross-section may appear to widen, form a neck or tapering in the center, then divide into two circular cross-sections, which represent the internal 201 and external 206 carotid arteries. The transducer 560 may have a line of sight indicator 561. As shown in FIG. 17 a percutaneous ablation device 562 (e.g. RF probe) may be inserted along the line of sight indicator 561 to the visualized carotid body, or location where a carotid body is expected to be (e.g. medial side of a saddle of the visualized carotid bifurcation), while safely avoiding the visualized carotid arteries. Optionally the transducer 560 may be placed at additional angles on the patient's neck, for example to obtain a sagittal view of the carotid arteries as shown in FIG. 18, to provide additional information of location of a percutaneous ablation device 562 relative to arteries or a carotid body. Alternative transducers and techniques may be used to place a percutaneous ablation device 562 safely proximate to a carotid body 101. The percutaneous ablation device 562 may optionally comprise an echogenic design. For example, the device may comprise a textured or grooved surface to increase backscatter of ultrasound waves to the transducer/transceiver, which may improve the visibility of the device.

Alternatively, prior to inserting a percutaneous ablation device to a target site a percutaneous device such as a needle, micro-needle, or electrical stimulation electrode may be placed proximate the target using ultrasound visualization.

It is understood that techniques for using ultrasound to assist percutaneous ablation of a carotid body or carotid body nerves while sparing other nerves, baroreceptors and vessels such as a jugular vein, are applicable to previously described endovascular ablation techniques. It is understood that different modalities of imaging may be combined and that endovascular catheter manipulations do not exclude, but potentially can compliment, percutaneous techniques.

Once the target carotid body or an estimated location of a carotid body such as the medial side of the saddle or bifurcation of carotid artery is identified using visualization, or possibly confirmed using electric stimulation, a percutaneous ablation device may be inserted into or placed proximate to the target. For example, as shown in FIGS. 14 to 16 the percutaneous ablation device may be a radiofrequency ablation needle 500 or cannula. RF ablation may be a desirable energy modality for ablating a carotid body or associated nerves because lesion volume may be controlled. Furthermore, a RF lesion formed proximate a carotid body may effectively ablate a carotid body or associated nerves while safely avoiding undesired or excessive damage of larger vessels such as the common, internal and eternal carotid arteries or jugular veins due to the heat sink in the vessel wall created by significant blood flow. A desirable lesion volume (e.g. about 30 to 900 mm³) may be achieved by selecting an RF electrode size (e.g. gauge and length), as well as RF energy delivery parameters (e.g. power, duration, and ramp slope). The RF ablation needle may be, for example equal to or smaller than about a 16 gauge needle (e.g. 20 gauge, 22 gauge) and may be electrically insulated along its shaft 502 with an electrically exposed distal tip 504 that is equal to or less than about 10 mm long (e.g. 2 to 5 mm). The RF ablation needle 500 may include a temperature sensor 506 in the distal tip 504. The RF ablation needle and temperature sensor may be connected to a computerized RF generator 510 via connection wires 509. A reference electrode 512 may be placed on the patient's skin to complete the RF circuit. The computerized RF generator 510 can control the delivery of RF energy to the RF ablation needle 500 using a feedback control algorithm 110 incorporating temperature data from the temperature sensor 506. Optionally, a RF ablation needle may have a blunt or rounded tip to reduce the risk of puncturing a carotid artery or jugular vein. A blunt tip RF ablation needle may be inserted through a small incision made with a scalpel or introducer needle in a patient's skin. Alternatively, a RF probe system may comprise a cannula with a square cut tip and a sharp trocar (e.g. pencil point, or beveled tip) that protrudes from the cannula to puncture through tissues such as the skin or carotid fascia. The sharp trocar may be replaced with a blunt tip trocar, stimulation electrode, or RF electrode when approaching or maneuvering proximate a carotid artery or carotid body (e.g. target site). Yet another alternative to a sharp trocar is to use a RF perforation electrode protruding from the cannula. RF perforation (e.g. perforation through electroporation) may be performed by delivering RF perforation parameters provided by a RF generator. For example, the RF energy may be configured to operate at high impedance (e.g. 2000-600052), low power (e.g. 5-25 W), high voltage (e.g. 150-180V) at short pulses (e.g. 0.25-3 seconds). Such RF parameters delivered through a relatively small electrode may cause tissue cells to rupture allowing the electrode to gently push through tissue. A benefit of passing through tissue such as carotid fascia using RF perforation is that less physical force is required and tenting (a phenomenon of a tough tissue resisting puncture of a sharp object and deforming to the object in a tent shape until enough force is applied to break through the tissue) is greatly reduced. Thus a risk of puncturing through a carotid fascia with a sharp device and inadvertently advancing the sharp device too far or uncontrolled into a carotid artery is greatly reduced. RF perforation energy may be turned off or the RF perforation electrode may be replaced by a blunt tip or sharp tip trocar or electrode in the cannula to pass the device through softer tissue.

Additionally, once the percutaneous ablation device is inserted to a desired location and prior to applying ablative energy, stimulation current can be applied to the carotid body to confirm the location of the ablation device. For example stimulation current can be current known to excite afferent sympathetic nerve fibers (e.g. 20 Hz, 100 μs pulse, 1-10 mA). A sympathetic physiological response (e.g. increased blood pressure, respiration or heart rate) could indicate that the instrument is at the right location and ablation energy may then be applied. Negative responses that will suggest that a different location can be sought will be reduction of HR and BP (caused by baroreflex or vagal nerve stimulation), protrusion of the tongue, or twitch of facial or throat muscles (caused by hypoglossal nerve stimulation), or expected clinical signs of sympathetic activation (caused by sympathetic trunk stimulation).

Local anesthesia may interfere with a study of reflexes (e.g. electrical or chemical stimulation of a chemoreflex) by disabling all nerves in the area of infiltration by local anesthetic. Sedation with IV anesthetic or general anesthesia can be used to manage patient discomfort while permitting physiologic response to chemoreflex stimulation. Alternatively, local anesthesia can be implemented after the reflexes and location of carotid body is confirmed with a very small needle or electrical stimulation electrode (e.g. 20 to 25 gauge), which may be less painful to the patient than a larger percutaneous ablation device. For example, a small electrical stimulation electrode may be configured with a lumen and be inserted to a target site, stimulation may be applied and, upon confirmation of proximity to a carotid body via physiological response monitoring, local anesthetic and optionally contrast solution may be injected through the small electrical stimulation electrode lumen, which may allow a larger percutaneous ablation device to be inserted with minimal pain.

Another embodiment of confirming proximity to a carotid body prior to applying ablative energy makes use of a high concentration of somatostatin receptor sites in carotid bodies. A small amount of radioactive agent with a short half-life such as Indium-111 labeled somatostatin or commercially available Indium In-111 pentetreotide can be injected into a patient's blood stream or directly into a carotid artery. Indium In-111 pentetreotide is currently used for the scintigraphic localization of primary and metastatic neuroendocrine tumors bearing somatostatin receptors. CT scan scintigraphy is expected to allow for a high degree of accuracy in localizing carotid body. This or similar substances can be injected intravenously as commonly used in clinical practice. In addition, previously described techniques for EC occlusion (see FIG. 13) with balloons can assist injection of localization agents such as iodine based radiocontrast, gadolinium or In-111 pentetreotide as well as to reduce the amount of radioactive material required. The occlusion or partial occlusion technique will increase dwell time and penetration of such agents into the carotid body.

Alternatively, a different form of percutaneous ablation device may be used to deliver an ablative energy to ablate targeted tissue. For example, a cryogenic probe, thermal probe, or needle for injection of a chemical or ablating or sclerosing agent may be introduced through the skin of a patient's neck proximate to the carotid body as a percutaneous ablation technique. Alternatively, a cannula (e.g. a hollow needle) may be used to provide percutaneous access through tissue to introduce an ablation probe or catheter with electrodes or a drug infusion catheter.

Another embodiment of a percutaneous approach may involve using magnetic resonance imaging (MRI) to visualize a targeted carotid body and carotid arteries. A MRI antenna may be placed in the adjacent artery or vein or on the external surface of a patient's neck proximate to a patient's carotid body to increase resolution. A percutaneous ablation device made from MRI compatible material such as Nitinol may be used to inject an ablative solution or to apply RF energy. Since RF generators are high impedance circuits they do not interfere with MRI.

Another alternative embodiment may ablate a carotid body using energy applied from outside the patient's body. For example, systems employing extracorporeal High Intensity Focused Ultrasound (HIFU) or stereotactic radiotherapy may be used to focus ablative energy in targeted tissue. The targeted tissue may be identified and tracked using a non-invasive technology such as CT, MRI or sonography. Alternatively, a technique for visualization/identification that is described later may be employed.

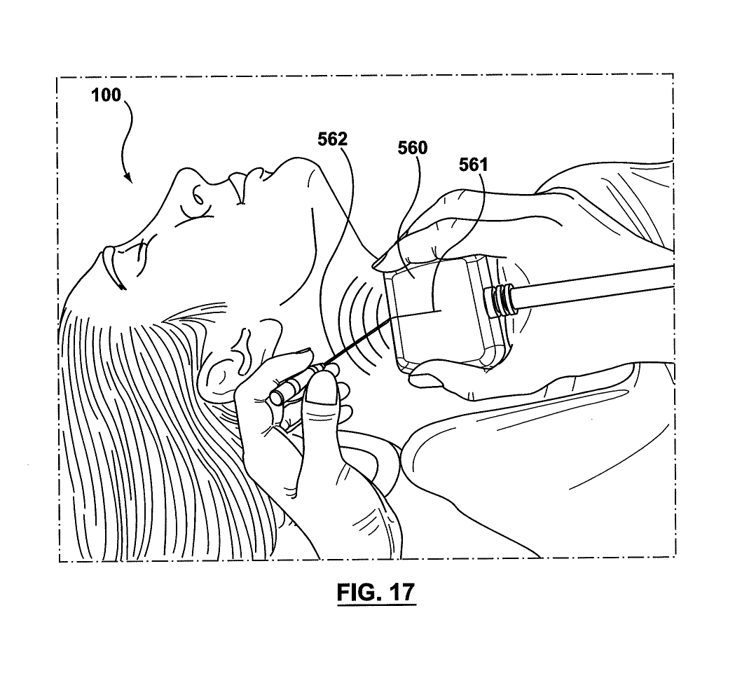

HIFU is a technology involving an ultrasound transducer (or array of transducers) positioned external to the body that focuses ultrasound beams, via a lens, a curved shape of the transducer, or dynamic control of a phased array of transducers), on a small volume of tissue a given depth from the surface. The focused beams heat tissue in the focal zone to an ablation temperature (e.g. 65 to 85° C.). As shown in FIG. 19, a MTV transducer 620 may be placed external a patient 100 and aimed at a carotid body 101. Ultrasound beams travel through a conductive medium 622 such as water and through the patient 100 where they are focused on a target site (e.g. carotid body) while sparing significant undesired damage to other tissues (e.g. jugular vein 108, internal carotid artery 201, external carotid artery 206). Locating the target site may be assisted using imaging technology such as MRI, CT or sonography. Optionally, prior to, or during ablation the transducer 620 may deliver ultrasound waves that do not heat tissue but create agitation. This could be used to stimulate the carotid body and confirm that the transducer is aimed at the correct location through monitoring physiological reactions. Following ablation agitation can confirm that the carotid body is ablated if it is no longer stimulated. Physiological monitors (e.g. heart rate monitor, blood pressure monitor, blood flow monitor, MSNA monitor) may communicate with a computerized HIFU generator to provide feedback information in response to stimulation. If a physiological response correlates to a given stimulation the computerized HIFU generator may provide an indication of a positive confirmation.

A method in accordance with a particular embodiment includes ablating at least one of a patient's carotid bodies based at least in part on identifying the patient as having a sympathetically mediated disease such as cardiac, metabolic, or pulmonary disease such as hypertension (e.g. refractory hypertension), congestive heart failure (CHF), or dyspnea.

A procedure may include diagnosis, selection based on diagnosis, further screening (e.g. baseline assessment of chemosensitivity), treating a patient based at least in part on diagnosis or further screening via a chemoreceptor (e.g. carotid body) ablation procedure such as one of the embodiments disclosed. Additionally, following ablation a method of therapy may involve conducting a post-ablation assessment to compare with the baseline assessment and making decisions based on the assessment (e.g. adjustment of drug therapy, re-treat in new position or with different parameters, or ablate a second chemoreceptor if only one was previously ablated).

A chemoreceptor ablation procedure may comprise the following steps or a combination thereof: patient sedation, locating a target peripheral chemoreceptor, visualizing a target peripheral chemoreceptor, confirming a target ablation site is or is proximate a peripheral chemoreceptor, confirming a target ablation site is safely distant from a baroreceptor or its nerves, providing stimulation (e.g. electrical, mechanical, chemical) to a target site or target peripheral chemoreceptor prior to, during or following an ablation step, monitoring physiological responses to said stimulation, anesthetizing a target site, protecting the brain from potential embolism, thermally protecting a proximate baroreceptor, ablating a target site or peripheral chemoreceptor, monitoring ablation parameters (e.g. temperature, impedance, blood flow in a carotid artery), confirming a reduction of chemoreceptor activity (e.g. chemosensitivity, HR, blood pressure, ventilation, sympathetic nerve activity) during or following an ablation step, removing an ablation device, conducting a post-ablation assessment, repeating any steps of the chemoreceptor ablation procedure on another peripheral chemoreceptor in the patient. Patient screening, as well as post-ablation assessment may include physiological tests or gathering of information, for example, chemoreflex sensitivity, central sympathetic nerve activity, heart rate, heart rate variability, blood pressure, ventilation, production of hormones, peripheral vascular resistance, blood pH, blood PCO2, degree of hyperventilation, peak VO2, VE/VCO2 slope. Directly measured maximum oxygen uptake (more correctly pVO2 in heart failure patients) and index of respiratory efficiency VE/VCO2 slope has been shown to be a reproducible marker of exercise tolerance in heart failure and provide objective and additional information regarding a patient's clinical status and prognosis.

A method of therapy may include electrical stimulation of a target region, using a stimulation electrode, to confirm proximity to a carotid body. For example, a stimulation signal having a 1-10 milliamps (mA) pulse train at about 20 to 40 Hz with a pulse duration of 50 to 500 microseconds (μs) that produces a positive carotid body stimulation effect may indicate that the stimulation electrode is within sufficient proximity to the carotid body or nerves of the carotid body to effectively ablate it. A positive carotid body stimulation effect could be increased blood pressure, heart rate, or ventilation concomitant with application of the stimulation. These variables could be monitored, recorded, or displayed to help assess confirmation of proximity to a carotid body. A catheter-based technique, for example, may have a stimulation electrode proximal to the energy delivery element used for ablation. Alternatively, the energy delivery element itself may also be used as a stimulation electrode. FIG. 7 can illustrate an embodiment of this concept where a radiofrequency electrode 207 may also be used to deliver a stimulation signal. In this case, the RF generator 210 or a separate stimulation generator may provide the stimulation signal to the electrode 207. Alternatively, an energy delivery element that delivers a form of ablative energy that is not electrical, such as a cryogenic ablation applicator, may be configured to also deliver an electrical stimulation signal as described earlier. Yet another alternative comprises a stimulation electrode that is distinct from an ablation element. For example, during a surgical procedure a stimulation probe can be touched to a suspected carotid body that is surgically exposed. A positive carotid body stimulation effect could confirm that the suspected structure is a carotid body and ablation can commence. Physiological monitors (e.g. heart rate monitor, blood pressure monitor, blood flow monitor, MSNA monitor) may communicate with a computerized stimulation generator, which may also be an ablation generator, to provide feedback information in response to stimulation. If a physiological response correlates to a given stimulation the computerized generator may provide an indication of a positive confirmation.

Alternatively or in addition a drug known to excite the chemo sensitive cells of the carotid body can be injected directly into the carotid artery or given systemically into patients vein or artery in order to elicit hemodynamic or respiratory response. Examples of drugs that may excite a chemoreceptor include nicotine, atropine, Doxapram, Almitrine, hyperkalemia, Theophylline, adenosine, sulfides, Lobeline, Acetylcholine, ammonium chloride, methylamine, potassium chloride, anabasine, coniine, cytosine, acetaldehyde, acetyl ester and the ethyl ether of i-methylcholine, Succinylcholine, Piperidine, monophenol ester of homo-iso-muscarine and acetylsalicylamides, alkaloids of veratrum, sodium citrate, adenosinetriphosphate, dinitrophenol, caffeine, theobromine, ethyl alcohol, ether, chloroform, phenyldiguanide, sparteine, coramine (nikethamide), metrazol (pentylenetetrazol), iodomethylate of dimethylaminomethylenedioxypropane, ethyltrimethylammoniumpropane, trimethylammonium, hydroxytryptamine, papaverine, neostigmine, acidity.

A method of therapy may further comprise applying electrical or chemical stimulation to the target area or systemically following ablation to confirm a successful ablation. Heart rate, blood pressure or ventilation may be monitored for change or compared to the reaction to stimulation prior to ablation to assess if the targeted carotid body was ablated. Post-ablation stimulation may be done with the same apparatus used to conduct the pre-ablation stimulation. Physiological monitors (e.g. heart rate monitor, blood pressure monitor, blood flow monitor, MSNA monitor) may communicate with a computerized stimulation generator, which may also be an ablation generator, to provide feedback information in response to stimulation. If a physiological response correlated to a given stimulation is reduced following an ablation compared to a physiological response prior to the ablation, the computerized generator may provide an indication ablation efficacy or possible procedural suggestions such as repeating an ablation, adjusting ablation parameters, changing position, ablating another carotid body or chemosensor, or concluding the procedure.

An optional step of visualizing internal structures (e.g. carotid body or surrounding structures) may be accomplished using one or more non-invasive imaging modalities (e.g. fluoroscopy, radiography, arteriography, CT, MRI, sonography) or minimally invasive techniques (e.g. IVUS, endoscopy, optical coherence tomography). A visualization step may be performed as part of a patient assessment, prior to an ablation procedure to assess risks and location of anatomical structures, during an ablation procedure to help guide an ablation device, or following an ablation procedure to assess outcome (e.g. efficacy of the ablation).

FIG. 13 shows a schematic view of blood vessels, such as small arteries 204 and 209 perfusing the carotid body, and the carotid sinus nerve 205. The carotid sinus nerve 205 carries signals from chemoreceptors in the carotid body 101 and baroreceptors in the carotid sinus 202.

Due to proximity to the wall of the CC and consistent (expected in at least 80% of the cases) position of the carotid body in humans the procedure may be guided visually using fluoroscopy assisted by radiographic contrast injections into the CC, IC, or EC.

Endovascular (for example transfemoral) arteriography of the common carotid and then selective arteriography of the internal and external carotids may be used to determine a position of a catheter tip at a CC bifurcation. Additionally, ostia of glomic arteries (these arteries may be up to 4 mm long and arise directly from the main parent artery) can be identified by dragging the dye injection catheter and releasing small amounts (“puffs”) of dye. If a glomic artery is identified it can be cannulated by a guide wire and possibly further cannulated by small caliber catheter. Direct injection of dye into glomic arteries can further assist the interventionalist in the ablation procedure. It is appreciated that the feeding glomic arteries are small and microcatheters may be needed to cannulate them.

Alternatively, ultrasound visualization may allow a physician to see the carotid arteries and even the carotid body. Another method for visualization may consist of inserting a small needle (e.g. 22 Gauge) with computer tomography (CT) guidance into or toward the carotid body. A wire or needle can be left in place as a fiducial guide, or contrast can be injected into the carotid body. Runoff of contrast to the jugular vein may confirm that the target is achieved.

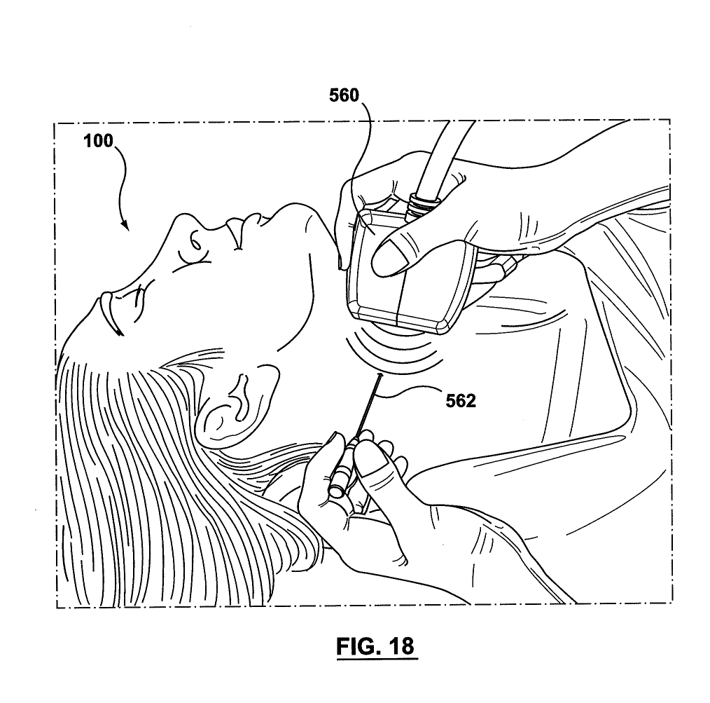

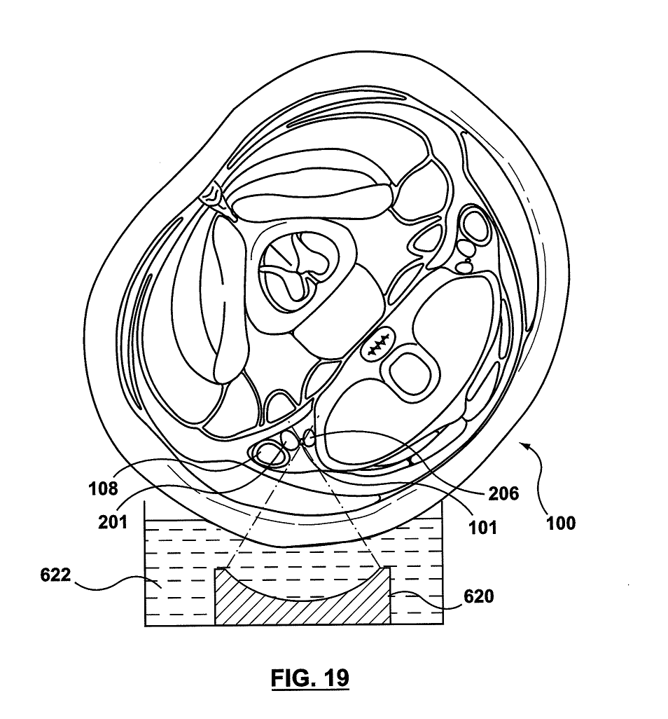

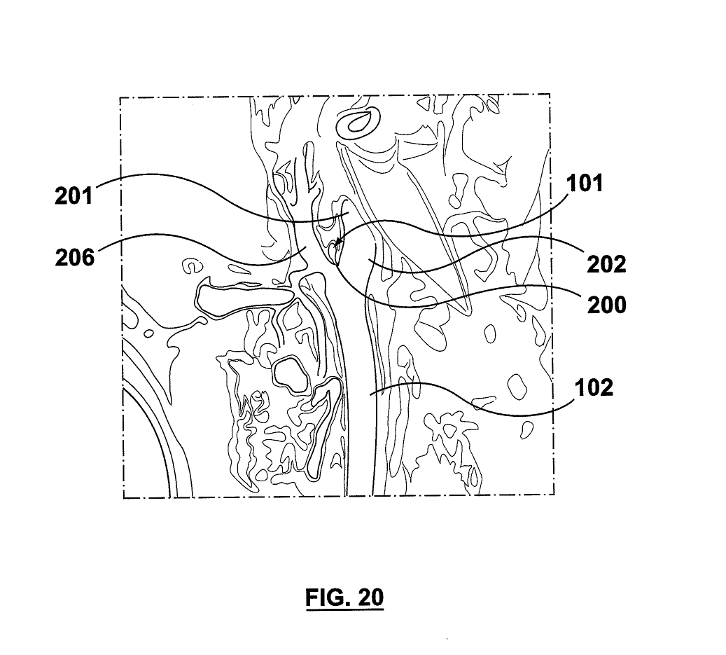

Computer Tomography (CT) may also be used to aid in identifying a carotid body. FIG. 20 (Nguyen R. P. Carotid Body Detection on CT Angiography, AM J Neuroradiology 32:1096-99, June-July 2011) is a CT image, performed with a B20 kernel at a 2 mm thickness at 2 mm increments, of an oblique sagittal view of subject's neck showing a carotid body at a carotid bifurcation. Such imaging could be used to help guide an ablation device to a carotid body or provide targeting for an extracorporeal ablation procedure (e.g. HIFU, stereotactic radiotherapy).

Ultrasound visualization (e.g. sonography) is an ultrasound-based imaging technique used for visualizing subcutaneous body structures including blood vessels and surrounding tissues. Doppler ultrasound uses reflected ultrasound waves to identify and display blood flow through a vessel. Operators typically use a hand-held transducer/transceiver placed directly on a patient's skin and aimed inward directing ultrasound waves through the patient's tissue. Ultrasound may be used to visualize a patient's carotid body to help guide an ablation device.

Visualization and navigation steps may comprise multiple imaging modalities (e.g. CT, fluoroscopy, ultrasound) superimposed digitally to use as a map for instrument positioning. Superimposing borders of great vessels such as carotid arteries can be done to combine images.

Responses to stimulation at different coordinate points can be stored digitally as a 3-dimensional or 2-dimensional orthogonal plane map. Such an electric map of the carotid bifurcation showing points, or point coordinates that are electrically excitable such as baroreceptors, baroreceptor nerves, chemoreceptors and chemoreceptor nerves can be superimposed with an image (e.g. CT, fluoroscopy, ultrasound) of vessels. This can be used to guide the procedure, and identify target areas and areas to avoid.

In addition, as noted above, it should be understood that a device providing therapy can also be used to locate a carotid body as well as to provide various stimuli (electrical, chemical, other) to test a baseline response of the CBC/CSB and measure changes in these responses after therapy or a need for additional therapy to achieve the desired physiological and clinical effects.

Chemoreflex or afferent nerve activity of carotid body nerves cannot be directly measured in a practical way, thus indexes of chemoreflex such as chemosensitivity can sometimes be used instead. Chemoreflex reduction is generally indicated by a reduction of an increase of ventilation and ventilation effort per unit of blood gas concentration, saturation or partial pressure change or by a reduction of CSNA that can be measured indirectly. Sympathetic nerve activity can be assessed by measuring activity of peripheral nerves leading to muscles (MSNA), heart rate (HR), heart rate variability (HRV), production of hormones such as renin, epinephrine and angiotensin, and peripheral vascular resistance. All these parameters are measurable and can lead directly to the health improvements. In the case of CHF patients' blood pH, blood PCO₂, degree of hyperventilation and metabolic exercise test parameters such as peak VO₂, and VE/VCO₂ slope are also important. It is believed that patients with heightened chemoreflex have low VO₂ and high VE/VCO₂ slope (index of respiratory efficiency) as a result of, for example, tachypnea and low blood CO₂. These parameters are also related to exercise limitations that further speed up patient's status deterioration towards morbidity and death. It is understood that all these indexes are indirect and imperfect and intended to direct therapy to patients that are most likely to benefit or to acquire an indication of technical success of ablation rather than to prove an exact measurement of effect or guarantee a success. It has been observed that some tachyarrhythmias in cardiac patients are sympathetically mediated. Thus carotid body ablation may be instrumental in treating reversible atrial fibrillation and ventricular tachycardia.

In an embodiment, a procedure may comprise assessing a patient to be a plausible candidate for carotid body ablation, additional details of which are described above. Such assessment may involve diagnosing a patient with a sympathetically mediated disease (e.g. MSNA microneurography, measure of cataclomines in blood or urine, heart rate, or low/high frequency analysis of heart rate variability may be used to assess sympathetic tone). Patient assessment may further comprise other patient selection criteria, for example indices of high carotid body activity (i.e. carotid body hypersensitivity or hyperactivity) such as a combination of hyperventilation and hypocarbia at rest, high carotid body nerve activity (e.g. measured directly), incidence of periodic breathing, dyspnea, central sleep apnea elevated brain natriuretic peptide, low exercise capacity, having cardiac resynchronization therapy, atrial fibrillation, ejection fraction of the left ventricle, using beta blockers or ACE inhibitors. Patient assessment may further involve selecting patients with high peripheral chemosensitivity (e.g. a respiratory response to hypoxia normalized to the desaturation of oxygen greater than or equal to about 0.7 l/min/min SpO₂), which may involve characterizing a patient's chemoreceptor sensitivity, reaction to temporarily blocking carotid body chemoreflex, or a combination thereof.

Although there are many ways to measure chemosensitivity they can be divided into (a) active provoked response and (b) passive monitoring. Active tests can be done by inducing intermittent hypoxia (such as by taking breaths of nitrogen or CO₂ or combination of gases) or by rebreathing air into and from a 4 to 10 liter bag. For example: a hypersensitive response to a short period of hypoxia measured by increase of respiration or heart rate may provide an indication for therapy. Ablation or significant reduction of such response could be indicative of a successful procedure. Also, electrical stimulation, drugs and chemicals (e.g. dopamine, lidocane) exist that can block or excite a carotid body when applied.

The location and baseline function of the desired area of therapy (including the carotid and aortic chemoreceptors and baroreceptors and corresponding nerves) may be determined prior to therapy by application of stimuli to the carotid body or other organs that would result in an expected change in a physiological or clinical event such as an increase or decrease in SNS activity, heart rate or blood pressure. These stimuli may also be applied after the therapy to determine the effect of the therapy or to indicate the need for repeated application of therapy to achieve the desired physiological or clinical effect(s). The stimuli can be either electrical or chemical in nature and can be delivered via the same or another catheter or can be delivered separately (such as injection of a substance through a peripheral IV to affect the CBC that would be expected to cause a predicted physiological or clinical effect).

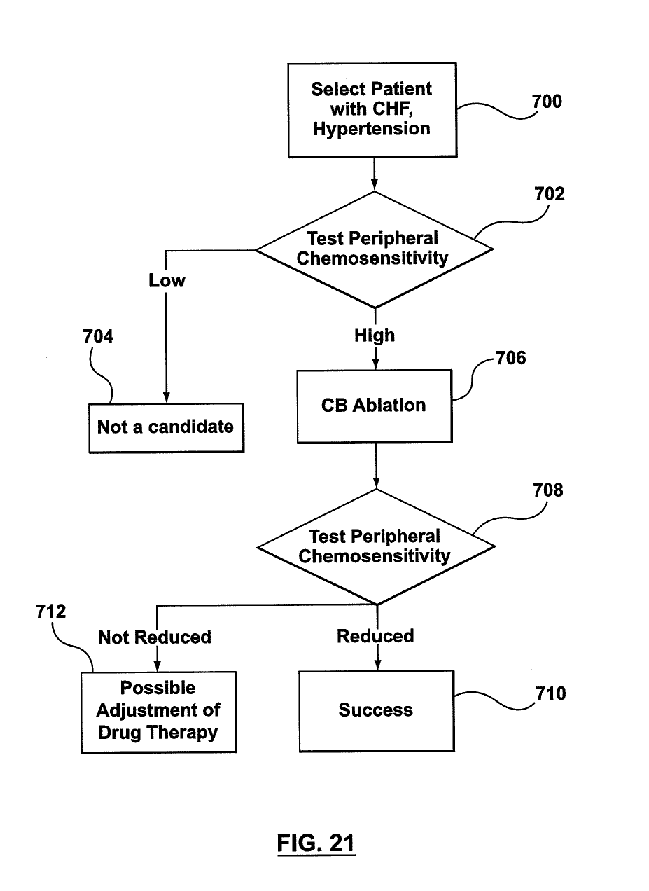

As shown in FIG. 21, a baseline stimulation test may be performed to select patients that may benefit from a carotid body ablation procedure. For example, patients with a high peripheral chemosensitivity gain (e.g. greater than or equal to about two standard deviations above an age matched general population chemosensitivity, or alternatively above a threshold peripheral chemosensitivity to hypoxia of 0.5 or 0.7 ml/min % O2) may be selected for a carotid body ablation procedure. A prospective patient suffering from a cardiac, metabolic, or pulmonary disease (e.g. hypertension, CHF, diabetes) may be selected 700. The patient may then be tested to assess a baseline peripheral chemoreceptor sensitivity (e.g. minute ventilation, tidal volume, ventilator rate, heart rate, or other response to hypoxic or hypercapnic stimulus) 702. Baseline peripheral chemosensitivity may be assessed using tests known in the art which involve inhalation of a gas mixture having reduced O₂ content (e.g. pure nitrogen, CO₂, helium, or breathable gas mixture with reduced amounts of O₂ and increased amounts of CO₂) or rebreathing of gas into a bag. Concurrently, the patient's minute ventilation or initial sympathetically mediated physiologic parameter such as minute ventilation or HR may be measured and compared to the O₂ level in the gas mixture. Tests like this may elucidate indices called chemoreceptor set-point and gain. These indices are indicative of chemoreceptor sensitivity. If the patient's chemosensitivity is not assessed to be high (e.g. less than about two standard deviations of an age matched general population chemosensitivity, or other relevant numeric threshold) then the patient may not be a suitable candidate for a carotid body ablation procedure 704. Conversely, a patient with chemoreceptor hypersensitivity (e.g. greater than or equal to about two standard deviations above normal) may proceed to have a carotid body ablation procedure 706. Following a carotid body ablation procedure the patient's chemosensitivity may optionally be tested again 708 and compared to the results of the baseline test 702. The second test 708 or the comparison of the second test to the baseline test may provide an indication of treatment success 710 or suggest further intervention 712 such as possible adjustment of drug therapy, repeating the carotid body ablation procedure with adjusted parameters or location, or performing another carotid body ablation procedure on a second carotid body if the first procedure only targeted one carotid body. It may be expected that a patient having chemoreceptor hypersensitivity or hyperactivity may return to about a normal sensitivity or activity following a successful carotid body ablation procedure.

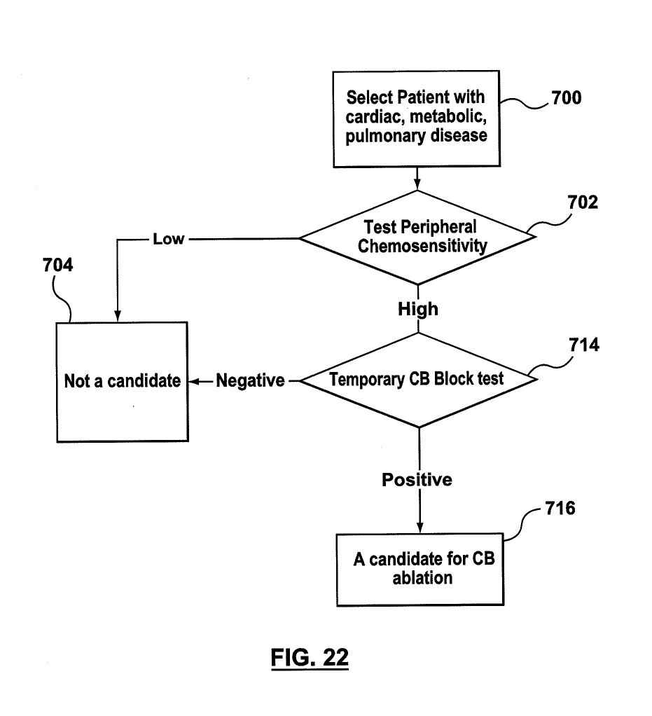

An alternative protocol for selecting a patient for a carotid body ablation procedure is shown in FIG. 22. Patients with high peripheral chemosensitivity or carotid body activity (e.g. ≧about 2 standard deviations above normal) alone or in combination with other clinical and physiologic parameters may be particularly good candidates for carotid body ablation therapy if they further respond positively to temporary blocking of carotid body activity. As shown in FIG. 22, a prospective patient suffering from a cardiac, metabolic, or pulmonary disease may be selected 700 to be tested to assess the baseline peripheral chemoreceptor sensitivity 702. A patient without high chemosensitivity may not be a plausible candidate 704 for a carotid body ablation procedure. A patient with a high chemosensitivity may be given a further assessment that temporarily blocks a carotid body chemoreflex 714. For example a temporary block may be done chemically, for example using a chemical such as intravascular dopamine or dopamine-like substances, intravascular alpha-2 adrenergic agonists, oxygen, in general alkalinity, or local or topical application of atropine externally to the carotid body. A patient having a negative response to the temporary carotid body block test (e.g. sympathetic activity index such as respiration, HR, heart rate variability, MSNA, vasculature resistance, etc. is not significantly altered) may be a less plausible candidate 704 for a carotid body ablation procedure. Conversely, a patient with a positive response to the temporary carotid body block test (e.g. respiration or index of sympathetic activity is altered significantly) may be a more plausible candidate for a carotid body ablation procedure 716.

There are a number of potential ways to conduct a temporary carotid body block test 714. Hyperoxia (e.g. higher than normal levels of PO₂) for example, is known to partially block (about a 50%) or reduce afferent sympathetic response of the carotid body. Thus, if a patient's sympathetic activity indexes (e.g. respiration, HR, HRV, MSNA) are reduced by hyperoxia (e.g. inhalation of higher than normal levels of O₂) for 3-5 minutes, the patient may be a particularly plausible candidate for carotid body ablation therapy. A sympathetic response to hyperoxia may be achieved by monitoring minute ventilation (e.g. reduction of more than 20-30% may indicate that a patient has carotid body hyperactivity). To evoke a carotid body response, or compare it to carotid body response in normoxic conditions, CO₂ above 3-4% may be mixed into the gas inspired by the patient (nitrogen content will be reduced) or another pharmacological agent can be used to invoke a carotid body response to a change of CO₂, pH or glucose concentration. Alternatively, “withdrawal of hypoxic drive” to rest state respiration in response to breathing a high concentration O₂ gas mix may be used for a simpler test.

An alternative temporary carotid body block test involves administering a sub-anesthetic amount of anesthetic gas halothane, which is known to temporarily suppress carotid body activity. Furthermore, there are injectable substances such as dopamine that are known to reversibly inhibit the carotid body. However, any substance, whether inhaled, injected or delivered by another manner to the carotid body that affects carotid body function in the desired fashion may be used.

Another alternative temporary carotid body block test involves application of cryogenic energy to a carotid body (i.e. removal of heat). For example, a carotid body or its nerves may be cooled to a temperature range between about −15° C. to 0° C. to temporarily reduce nerve activity or blood flow to and from a carotid body thus reducing or inhibiting carotid body activity.

An alternative method of assessing a temporary carotid body block test may involve measuring pulse pressure. Noninvasive pulse pressure devices such as Nexfin (made by BMEYE, based in Amsterdam, The Netherlands) can be used to track beat-to-beat changes in peripheral vascular resistance. Patients with hypertension or CHF may be sensitive to temporary carotid body blocking with oxygen or injection of a blocking drug. The peripheral vascular resistance of such patients may be expected to reduce substantially in response to carotid body blocking. Such patients may be good candidates for carotid body ablation therapy.

Yet another index that may be used to assess if a patient may be a good candidate for carotid body ablation therapy is increase of baroreflex, or baroreceptor sensitivity, in response to carotid body blocking. It is known that hyperactive chemosensitivity suppresses baroreflex. If carotid body activity is temporarily reduced the carotid sinus baroreflex (baroreflex sensitivity (BRS) or baroreflex gain) may be expected to increase. Baroreflex contributes a beneficial parasympathetic component to autonomic drive. Depressed BRS is often associated with an increased incidence of death and malignant ventricular arrhythmias. Baroreflex is measurable using standard non-invasive methods. One example is spectral analysis of RR interval of ECG and systolic blood pressure variability in both the high- and low-frequency bands. An increase of baroreflex gain in response to temporary blockade of carotid body can be a good indication for permanent therapy. Baroreflex sensitivity can also be measured by heart rate response to a transient rise in blood pressure induced by injection of phenylephrine.

An alternative method involves using an index of glucose tolerance to select patients and determine the results of carotid body blocking or removal in diabetic patients. There is evidence that carotid body hyperactivity contributes to progression and severity of metabolic disease.

In general, a beneficial response can be seen as an increase of parasympathetic or decrease of sympathetic tone in the overall autonomic balance. For example, Power Spectral Density (PSD) curves of respiration or HR can be calculated using nonparametric Fast Fourier Transform algorithm (FFT). FFT parameters can be set to 256-64 k buffer size, Hamming window, 50% overlap, 0 to 0.5 or 0.1 to 1.0 Hz range. HR and respiratory signals can be analyzed for the same periods of time corresponding to (1) normal unblocked carotid body breathing and (2) breathing with blocked carotid body.

Power can be calculated for three bands: the very low frequency (VLF) between 0 and 0.04 Hz, the low frequency band (LF) between 0.04-0.15 Hz and the high frequency band (HI) between 0.15-0.4 Hz. Cumulative spectral power in LF and HF bands may also be calculated; normalized to total power between 0.04 and 0.4 Hz (TF=HF+LF) and expressed as % of total. Natural breathing rate of CHF patient, for example, can be rather high, in the 0.3-0.4 Hz range.

The VLF band may be assumed to reflect periodic breathing frequency (typically 0.016 Hz) that can be present in CHF patients. It can be excluded from the HF/LF power ratio calculations.

The powers of the LF and HF oscillations characterizing heart rate variability (HRV) appear to reflect, in their reciprocal relationship, changes in the state of the sympathovagal (sympathetic to parasympathetic) balance occurring during numerous physiological and pathophysiological conditions. Thus, increase of HF contribution in particular can be considered a positive response to carotid body blocking.

Another alternative method of assessing carotid body activity comprises nuclear medicine scanning, for example with ocretide, somatostatin analogues, or other substances produced or bound by the carotid body.

Furthermore, artificially increasing blood flow may reduce carotid body activation. Conversely artificially reducing blood flow may stimulate carotid body activation. This may be achieved with drugs known in the art to alter blood flow.

There is a considerable amount of scientific evidence to demonstrate that hypertrophy of a carotid body often accompanies disease. A hypertrophied (i.e. enlarged) carotid body may further contribute to the disease. Thus identification of patients with enlarged carotid bodies may be instrumental in determining candidates for therapy. Imaging of a carotid body may be accomplished by angiography performed with radiographic, computer tomography, or magnetic resonance imaging.

It should be understood that the available measurements are not limited to those described above. It may be possible to use any single or a combination of measurements that reflect any clinical or physiological parameter effected or changed by either increases or decreases in carotid body function to evaluate the baseline state, or change in state, of a patient's chemosensitivity.

There is a considerable amount of scientific evidence to demonstrate that hypertrophy of a carotid body often accompanies disease. A hypertrophied or enlarged carotid body may further contribute to the disease. Thus identification of patients with enlarged carotid bodies may be instrumental in determining candidates for therapy.

Further, it is possible that although patients do not meet a preselected clinical or physiological definition of high peripheral chemosensitivity (e.g. greater than or equal to about two standard deviations above normal), administration of a substance that suppresses peripheral chemosensitivity may be an alternative method of identifying a patient who is a candidate for the proposed therapy. These patients may have a different physiology or co-morbid disease state that, in concert with a higher than normal peripheral chemosensitivity (e.g. greater than or equal to normal and less than or equal to about 2 standard deviations above normal), may still allow the patient to benefit from carotid body ablation. The proposed therapy may be at least in part based on an objective that carotid body ablation will result in a clinically significant or clinically beneficial change in the patient's physiological or clinical course. It is reasonable to believe that if the desired clinical or physiological changes occur even in the absence of meeting the predefined screening criteria, then therapy could be performed.

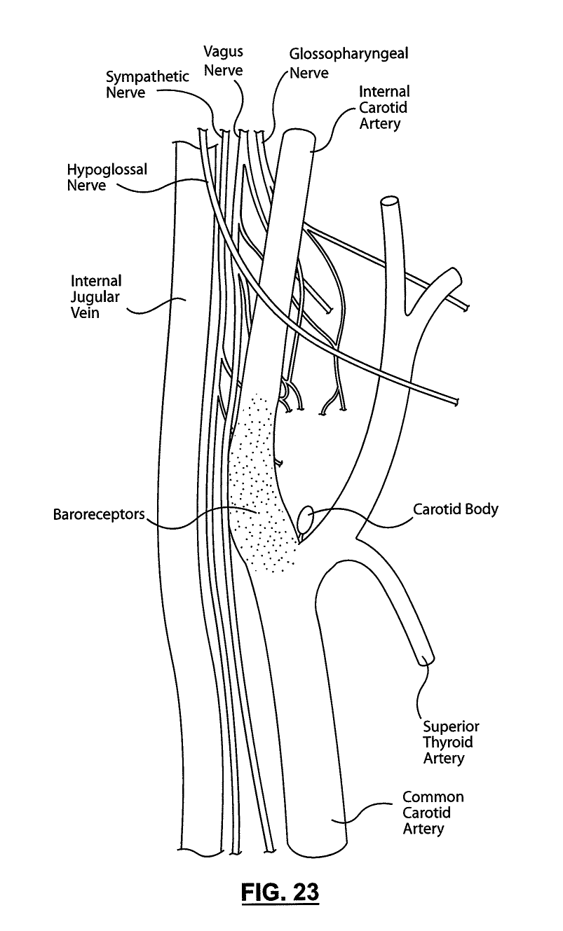

The carotid body (CB) (see FIG. 23), the dominant peripheral chemoreceptor in humans, responds primarily to acute hypoxemia, increases of arterial carbon dioxide tension (pCO₂), acidotic pH and hypoperfusion. The carotid bodies are 2.5-7 mm ovoid bilateral organs located at the bifurcation of each common carotid and are innervated by the nerve fibers from the glossopharyngeal, vagal and the sympathetic nerve of the nearby superior cervical ganglion. The CB is the most perfused organ per gram weight in the body (2000 ml/min/100 mg tissue) and receives blood via an arterial branch arising from internal or external carotid artery. Proposed underlying mechanisms for hyperactivity of the chemoreceptors are local alterations in perfusion, inflammation and changes in ASIC/TASC channels. The CB modulates systemic sympathetic tone through direct signaling to the part of the brain called medulla oblongata; resulting in an increase in blood pressure, and minute ventilation. Separately, the carotid baroreflex originates primarily from the carotid sinus, an outpouching of the internal carotid artery, and houses mechanoreceptors, which buffer acute changes in blood pressure through modulation of both parasympathetic and sympathetic nervous systems. Additional baroreflex input to the brain comes from numerous mechanoreceptors including those found in walls of the internal, external and common carotid arteries, aorta, and kidney. Chemoreflex and baroreflex are linked in control of sympathetic tone and autonomic balance; chemoreflex mediates sympathoactivation and inhibition of the baroreflex.

Chemosensitivity can be clinically assessed by measuring the ventilatory response, changes of muscle sympathetic nerve activity, or physiologic changes in heart rate or blood pressure in response to either inhibition or stimulation of the peripheral chemoreceptor by manipulating inhaled gas mixtures. Transient or progressive hypoxia stimulate, whereas brief hyperoxia or low dose dopamine inhibits the peripheral chemoreflex. Carotid body hyperactivity can occur in the absence of increased chemosensitivity; however, increased chemosensitivity may be associated with carotid body hyperactivity.

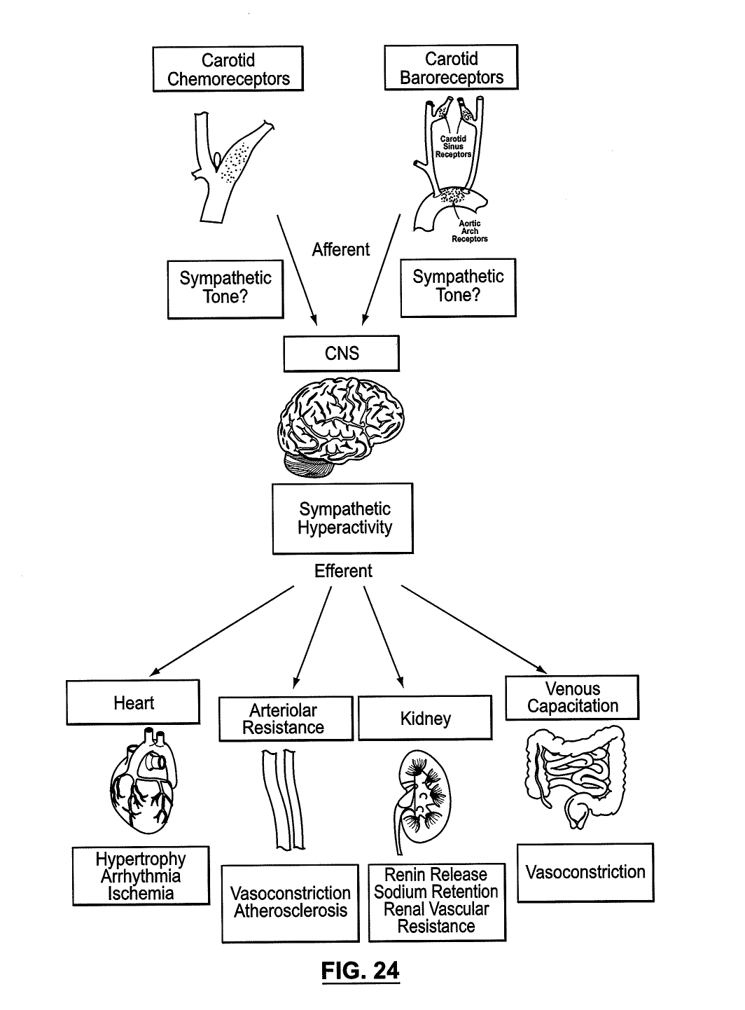

Many of the beneficial compensatory mechanisms activated in acute stress become maladaptive in chronic disease. We propose that chronic hyperactivity of the chemoreflex is maladaptive and leads to the development and progression of diseases impacted through chronic over stimulation of the sympathetic nervous system and inhibition of the protective baroreflex. As shown in FIG. 24 afferent nerve pathways from the carotid chemoreceptors communicate chronic hyperactivity of carotid body and peripheral chemoreflex to the central nervous system stimulating sympathetic hyperactivity, which is transmitted via efferent nerve pathways to organs, which may contribute to disease states.

Therapeutic reduction of hyperactive chemoreflex activity to reduce systemic sympathetic hyperactivity could favorably impact the morbidity and mortality in diseases noted for autonomic imbalance, including: heart failure with reduced ejection fraction (HFREF), and heart failure with preserved ejection fraction (HFREF), chronic and end stage renal disease, insulin resistance, Type II diabetes, Obesity, Central Sleep Apnea, sleep disorders, congestion and essential hypertension. Efferent Central Sympathetic Nerve Activity (CSNA) activity has been shown to predict outcome in these diseases.

A method has been conceived for therapeutic reduction of hyperactive chemoreflex and carotid body activity, herein referred to as Carotid Body Ablation (CBA). To be effective, carotid body ablation must result in the reduction of afferent nerve activity of a carotid body. Afferent nerves refer to the nerves connecting Carotid Body to the Central Nervous System (CNS). The reduction of afferent nerve activity of a carotid body may reduce CSNA. The reduction of CSNA may result in the reduction of efferent sympathetic nerve activity to important organs such as: kidneys, blood vessels and the heart. FIG. 24 shows components of the physiologic system that illustrates sympathetically mediated disease. Efferent sympathetic nerve activity refers to the sympathetic excitation signals conducted from CNS to the peripheral nervous system and organs of the body.

The reduction of efferent sympathetic nerve activity to important organs may lead to improvements of their function such as: reduction of cardiac arrhythmias, reduction of heart rate, vasodilation of blood vessels and reduction of blood pressure or loading of the heart muscle, remodeling of heart muscle, reduction of sodium retention by kidneys, reduction of deterioration of diseased kidneys, redistribution of blood flow and reduction of insulin resistance among others. These improvements may result in reduction of symptoms and reduced severity or even reversal of sympathetically mediated diseases, including hypertension, heart failure and diabetes.

Reduction of afferent nerve activity of carotid body and efferent sympathetic nerve activity to important organs may also result in a multitude of improvements in symptoms and the status of patients: reduction of insulin resistance, reduction of blood pressure, reduction of central sleep apnea, reduction of breathing rate and hyperventilation, reduction of heart rate, improved baroreflex, reduction of dyspnea, increase of exercise tolerance, reduction of sodium and fluid retention, reduction of hypertrophy of heart muscle, reduction of renal hyperfiltration and proteinuria, reduction of heart arrhythmias and many others.

These improvements of organ and whole body function may reverse or slow down the progression of sympathetically mediated diseases, improve quality of life of patients, reduce their pain and suffering, and extend life.

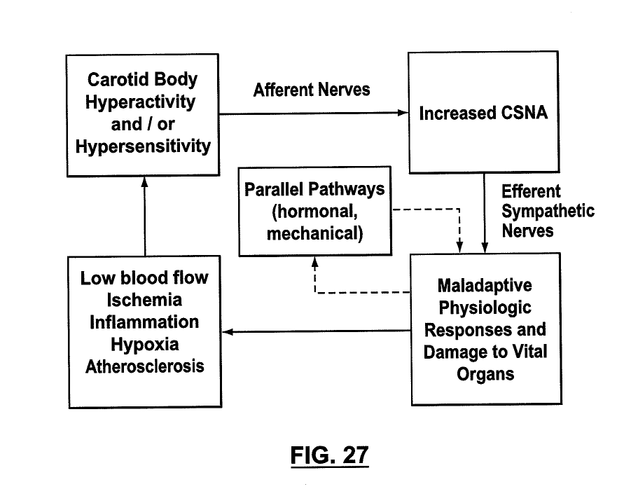

As shown in FIG. 27 carotid body hyperactivity or hypersensitivity is part of a cycle of disease progression. It is likely that the progression of disease worsens the hyperactivity of the carotid body via mechanisms such as ischemia, arthrosclerosis, hypoperfusion and inflammation. The increased carotid body hyperactivity worsens the disease and so on. Ablation of carotid body or interrupting the afferent neural pathway from the CB to the central nervous system may be a therapeutic treatment that interrupts this cycle.

The proposed therapy, broadly defined as carotid body ablation, is expected to result in a reduction of central sympathetic nerve activity by reducing afferent nerve activity of carotid body cells (e.g. glomus cells, chemosensory cells, afferent nerves) via ablation that causes sustained damage to these cells, afferent nerves from the cells, or blood vessels supplying blood to these nerves or cells. A method for carotid body ablation may comprise various approaches including surgical, keyhole surgical, endovascular, percutaneous, or extracorporeal. Ablation may be achieved via surgical resection, thermal, thermal cryogenic, chemical or mechanical tissue destruction. In all cases the afferent nerve signaling to the CNS and brain is substantially reduced. The proposed therapy may have several applications such as treatment to reduce symptoms or reverse or stop the progression of disease states described herein.

Carotid body hyperactivity increases central sympathetic nerve activity (CSNA) and thus contributes to hypertension (HTN) through direct increases in renal neurogenic sodium avidity and increases in renal renin-angiotensin-aldosterone system (RAAS) system activation as well as direct neurogenically mediated increases in vascular resistance. The physiologic significance of this has been explored in both preclinical and human trials.

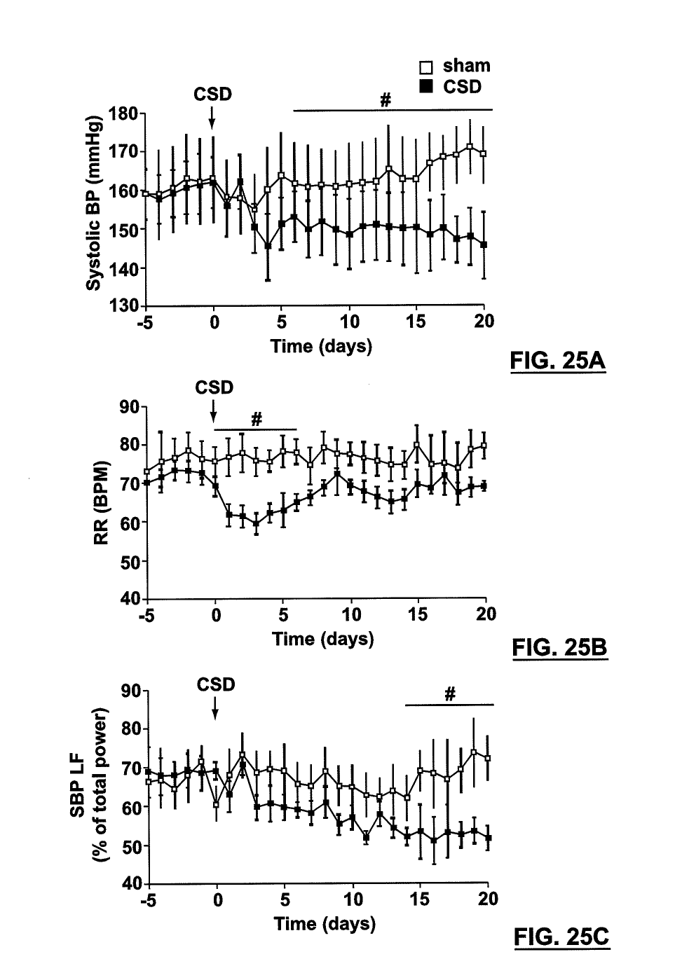

Under request of the inventors based on concepts disclosed herein, Abdala et al. have performed experiments and reported that interference with bilateral CB signaling, by interrupting its afferent nerves, results in significant blood pressure reduction in spontaneously hypertensive rats (SHR) and causing no changes of pressure in normotensive animals. Interestingly CB denervation caused also an improvement in baroreceptor function, potentially caused by a re-setting of the central baroreceptor control or by the removal of direct neural suppression of baroreflex from carotid body (FIGS. 25A, 25B and 25C). Similar experiments when CB nerves were interrupted (ablated) unilaterally did not result in BP reduction.

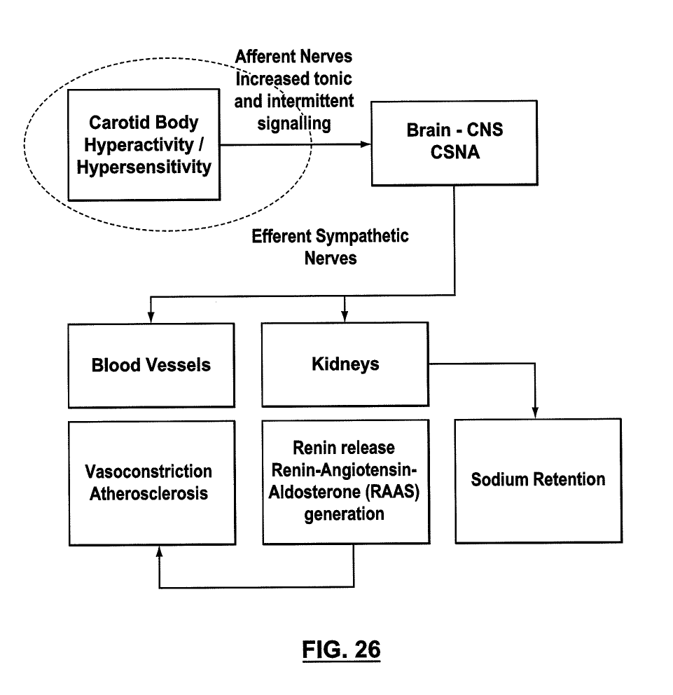

As shown in FIG. 26 hyperactive carotid body and heightened chemosensitivity can result in increased central sympathetic drive originating from the neurons in the brain medulla and thus contributes to hypertension through direct increases in neurogenically mediated renal sodium avidity and increases in renal Renin-Angiotensin-Aldosterone System (RAAS) activation as well as directly neurogenically mediated increases in peripheral vascular resistance.

Although a physiological connection between carotid body hyperactivity and hypertension has been observed in experimental rodents and removal of the carotid body to treat asthma has been seen to occasionally reduce blood pressure, no one has considered removal of the carotid body as part of a method for treating patients having hypertension.

At the request of Inventors clinical research investigators in Gdansk, Poland surgically removed one carotid body (on the right side) in a small group of human subjects with confirmed, long standing severe hypertension resistant to all tried drug therapies. After one month follow-up inventors were surprised to see dramatic and unprecedented reduction of blood pressure in the subgroup of patients where surgical removal of right side carotid body was confirmed by pathology. It was previously known to investigators that unilateral removal of carotid body in hypertensive rats does not reduce blood pressure.

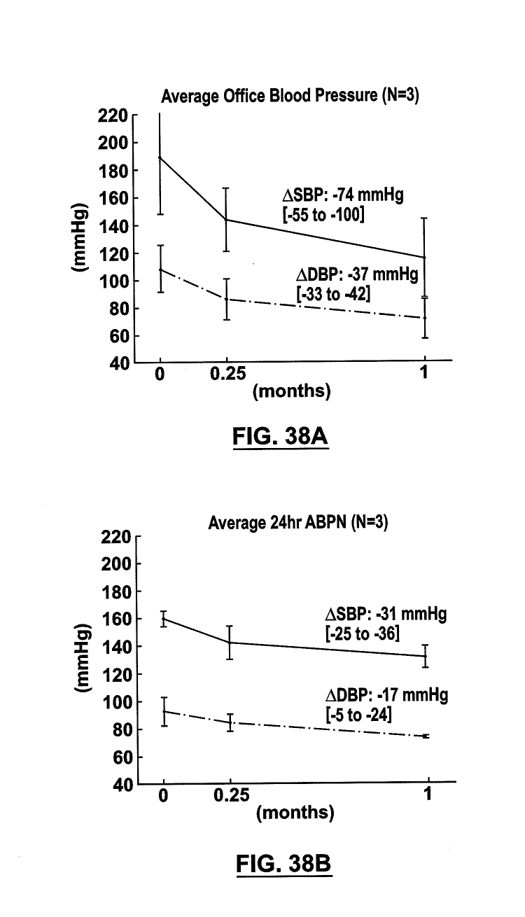

FIGS. 38A and 38B show the BP change (average and standard deviation, systolic and diastolic) results of unilateral surgical ablation of carotid body in three hypertensive patients in Gdansk. In all patients only the right side CB was removed. After one week and one month the blood pressure measured with a blood pressure cuff during doctor's office visit and with an ambulatory 24 hour wearable BP monitor (graph represents 24 hour average) is significantly reduced.

In chronic Congestive Heart Failure (CHF), the sympathetic nervous system activation that is directed to attenuate systemic hypoperfusion at the initial phases of CHF may ultimately exacerbate the progression of cardiac dysfunction that subsequently increases the extra-cardiac abnormalities, a positive feedback cycle of progressive deterioration, a vicious cycle with ominous consequences. It was thought that much of the increase in the sympathetic nerve activity (SNA) in CHF was based on an increase of sympathetic flow at a level of the CNS and on the depression of arterial baroreflex function. In the past several years, it has been demonstrated that an increase in the activity and sensitivity of peripheral chemoreceptors (heightened chemoreflex function) also plays an important role in the enhanced SNA that occurs in CHF.

Chronic elevation in sympathetic nerve activity (SNA) is associated with the development and progression of certain types of hypertension and contributes to the progression of congestive heart failure (CHF). It is also known that sympathetic excitatory cardiac, somatic, and central/peripheral chemoreceptor reflexes are abnormally enhanced in CHF and hypertension (Ponikowski, 2011 and Giannoni, 2008 and 2009).

As often happens in chronic disease states, chemoreflexes that are dedicated under normal conditions to maintaining homeostasis and correcting hypoxia contribute to increase the sympathetic tone in patients with CHF, even under normoxic conditions. The understanding of how abnormally enhanced sensitivity of the peripheral chemosensors, particularly the carotid body, contributes to the tonic elevation in SNA in patients with CHF has come from several studies in animals. According to one theory, the local angiotensin receptor system plays a fundamental role in the enhanced carotid body chemoreceptor sensitivity in CHF. In addition, evidence in both CHF patients and animal models of CHF has clearly established that the carotid body chemoreflex is often hypersensitive in CHF patients and contributes to the tonic elevation in sympathetic function. This derangement derives from altered function at the level of both the afferent and central pathways of the reflex arc. The mechanisms responsible for elevated afferent activity from the carotid body in CHF are not yet fully understood.

Regardless of the exact mechanism behind the carotid body hypersensitivity, the chronic sympathetic activation driven from the carotid body and other autonomic pathways leads to further deterioration of cardiac function in a positive feedback cycle. As CHF ensues, the increasing severity of cardiac dysfunction leads to progressive escalation of these alterations in carotid body chemoreflex function to further elevate sympathetic activity and cardiac deterioration. The trigger or causative factors that occur in the development of CHF that sets this cascade of events in motion and the time course over which they occur remain obscure. Ultimately, however, causative factors are tied to the cardiac pump failure and reduced cardiac output. According to one theory, within the carotid body, a progressive and chronic reduction in blood flow may be the key to initiating the maladaptive changes that occur in carotid body chemoreflex function in CHF.

There is sufficient evidence that there is increased peripheral and central chemoreflex sensitivity in many patients with heart failure, which is likely to be correlated with the severity of the disease. There is also some evidence that the central chemoreflex is modulated by the peripheral chemoreflex. According to current theories, the carotid body is the predominant contributor to the peripheral chemoreflex in humans; the aortic body having a minor contribution.

Although the mechanisms responsible for altered central chemoreflex sensitivity remain obscure, the enhanced peripheral chemoreflex sensitivity can be linked to a depression of nitric oxide production in the carotid body affecting afferent sensitivity, and an elevation of central angiotensin II affecting central integration of chemoreceptor input. The enhanced chemoreflex may be responsible, in part, for the enhanced ventilatory response to exercise, dyspnea, Cheyne-Stokes breathing, and sympathetic activation observed in chronic heart failure patients. The enhanced chemoreflex may be also responsible for hyperventilation and tachypnea (e.g. fast breathing) at rest and exercise, periodic breathing during exercise, rest and sleep, hypocapnia, vasoconstriction, reduced peripheral organ perfusion and hypertension.

Sympathetic activation is a seminal feature of chronic heart failure, underlying both initiation and progression of the syndrome. These elevations of sympathetic tone are linked to impairment of inhibitory baroreflex control of cardiovascular function. Increased excitatory input from peripheral chemoreceptors can contribute to sympathetic hyperactivity and cardiac baroreceptor dysfunction and poor outcome. Increases of peripheral chemoreflex sensitivity (chemosensitivity) directly decreases baroreceptor function in Congestive Heart Failure (CHF) patients, contributing to sympathetic overactivity.

At the request of the inventors based on concepts disclosed herein, experiments were performed and preclinical data has been gathered that links chemoreflex sensitivity and carotid body hyperactivity to CHF pathology. Chemoreflex sensitivity is enhanced in rabbits with rapid pacing-induced heart failure (HF). Nerve activity of CB chemoreceptors and renal sympathetic nerve activity (RSNA), both at rest and in response to hypoxia, is enhanced in a pacing model of heart failure in rabbits. In this model, hyperoxic inhibition of the chemoreflex reduces resting RSNA, documenting that the chemoreceptor hyperactivity underlies the systemic and renal specific sympathetic hyperactivity. This increased RSNA initiates the triad of renin release, sodium retention and reduced renal blood flow, all three documented components of the cardiorenal syndrome. Furthermore, CB denervation in this well accepted animal heart failure model results in attenuation of both resting RSNA and plasma norepinephrine levels. These animal data demonstrate that CB chemoreflex function is hyperactive in HF, and that excessive CB activity is sufficient to cause increases of systemic and renal specific sympathetic signaling; CB removal attenuates both systemic and RSNA.

### Dyspnea

Shortness of breath, or dyspnea, is a feeling of difficult or labored breathing that is out of proportion to the patient's level of physical activity. It is a symptom of a variety of different diseases or disorders and may be either acute or chronic. Dyspnea is the most common complaint of patients with cardiopulmonary diseases.

Dyspnea is believed to result from complex interactions between neural signaling, the mechanics of breathing, and the related response of the central nervous system. A specific area has been identified in the mid-brain that may influence the perception of breathing difficulties.

The experience of dyspnea depends on its severity and underlying causes. The feeling itself results from a combination of impulses relayed to the brain from nerve endings in the lungs, rib cage, chest muscles, or diaphragm, combined with the perception and interpretation of the sensation by the patient. In some cases, the patient's sensation of breathlessness is intensified by anxiety about its cause. Patients describe dyspnea variously as unpleasant shortness of breath, a feeling of increased effort or tiredness in moving the chest muscles, a panicky feeling of being smothered, or a sense of tightness or cramping in the chest wall.

The four generally accepted categories of dyspnea are based on its causes: cardiac, pulmonary, mixed cardiac or pulmonary, and non-cardiac or non-pulmonary. The most common heart and lung diseases that produce dyspnea are asthma, pneumonia, COPD, and myocardial ischemia or heart attack (myocardial infarction). Foreign body inhalation, toxic damage to the airway, pulmonary embolism, congestive heart failure (CHF), anxiety with hyperventilation (panic disorder), anemia, and physical deconditioning because of sedentary lifestyle or obesity can produce dyspnea. In most cases, dyspnea occurs with exacerbation of the underlying disease. Dyspnea also can result from weakness or injury to the chest wall or chest muscles, decreased lung elasticity, obstruction of the airway, increased oxygen demand, or poor pumping action of the heart that results in increased pressure and fluid in the lungs, such as in CHF.

Acute dyspnea with sudden onset is a frequent cause of emergency room visits. Most cases of acute dyspnea involve pulmonary (lung and breathing) disorders, cardiovascular disease, or chest trauma. Sudden onset of dyspnea (acute dyspnea) is most typically associated with narrowing of the airways or airflow obstruction (bronchospasm), blockage of one of the arteries of the lung (pulmonary embolism), acute heart failure or myocardial infarction, pneumonia, or panic disorder.

Chronic dyspnea is different. Long-standing dyspnea (chronic dyspnea) is most often a manifestation of chronic or progressive diseases of the lung or heart, such as COPD, which includes chronic bronchitis and emphysema. The treatment of chronic dyspnea depends on the underlying disorder. Asthma can often be managed with a combination of medications to reduce airway spasms and removal of allergens from the patient's environment. COPD requires medication, lifestyle changes, and long-term physical rehabilitation. Anxiety disorders are usually treated with a combination of medication and psychotherapy.

Although the exact mechanism of dyspnea in different disease states is debated, there is no doubt that the CBC plays some role in most manifestations of this symptom. Dyspnea seems to occur most commonly when afferent input from peripheral receptors is enhanced or when cortical perception of respiratory work is excessive.

Ablation of a peripheral chemoreceptor (e.g. carotid body) in patients having sympathetically mediated disease and high peripheral chemosensitivity has been conceived to reduce peripheral chemosensitivity and consequentially reduce afferent signaling from peripheral chemoreceptors to the central nervous system. The expected reduction of chemoreflex sensitivity to hypoxia and other stimuli such as blood flow, blood CO₂, glucose concentration or blood pH can directly reduce afferent signals from chemoreceptors and produce at least one beneficial effect such as the reduction of central sympathetic activation, reduction of the sensation of breathlessness (dyspnea), vasodilation, increase of exercise capacity, reduction of blood pressure, reduction of sodium and water retention, reduction of insulin resistance, reduction of hyperventilation, reduction of tachypnea, reduction of hypocapnia, or improve symptoms of a sympathetically mediated disease and may ultimately slow down the disease progression and extend life. It is understood that a sympathetically mediated disease that may be treated with carotid body ablation may comprise elevated sympathetic tone, an elevated sympathetic/parasympathetic activity ratio, autonomic imbalance primarily attributable to central sympathetic tone being abnormally high, or heightened sympathetic tone at least partially attributable to afferent excitation traceable to hypersensitivity or hyperactivity of a peripheral chemoreceptor (e.g. carotid body). In some important clinical cases where baseline hypocapnea is present, reduction of hyperventilation may be expected. It is understood that hyperventilation in the context of this disclosure means respiration in excess of metabolic needs on the individual that generally leads to slight but significant hypocapnea (blood CO₂ partial pressure below normal of approximately 40 mmHg, for example in the range of 33 to 38 mmHg).

Patients having CHF or hypertension concurrent with heightened peripheral chemoreflex sensitivity often react as if their system was hypercapnic even if it is not. The reaction is to hyperventilate, a maladaptive attempt to rid the system of CO₂, thus overcompensating and creating a hypocapnic and alkalotic system. Some researchers attribute this hypersensitivity/hyperactivity of the carotid body to the direct effect of catecholamines, hormones circulating in excessive quantities in the blood stream of CHF patients. The present disclosure may be particularly useful to treat such patients who are hypocapnic and possibly alkalotic resulting from high tonic output from carotid bodies. Such patients are particularly predisposed to periodic breathing and central apnea hypopnea type events that cause arousal, disrupt sleep, cause intermittent hypoxia and are by themselves detrimental and difficult to treat.

It is appreciated that periodic breathing of Cheyne Stokes pattern occurs in patients during sleep, exercise and even at rest as a combination of central hypersensitivity to CO₂, peripheral chemosensitivity to O₂ and CO₂ and prolonged circulatory delay. All these parameters are often present in CHF patients that are at high risk of death. Thus, patients with hypocapnea, CHF, high chemosensitivity and prolonged circulatory delay, and specifically ones that exhibit periodic breathing at rest or during exercise or induced by hypoxia are likely beneficiaries of the proposed therapy.

Some researchers believe that hyperventilation during sleep initiates and sustains periodic breathing and central sleep apnea. Inventors propose that removal of stimulus to hyperventilate that comes from carotid bodies may therefore stabilize breathing.

Hyperventilation is defined as breathing in excess of a person's metabolic need at a given time and level of activity. Hyperventilation is more specifically defined as minute ventilation in excess of that needed to remove CO₂ from blood in order to maintain blood CO₂ in the normal range (e.g. around 40 mmHg partial pressure). For example, patients with arterial blood PCO₂ in the range of 32-37 mmHg can be considered hypocapnic and in hyperventilation.

For the purpose of this disclosure hyperventilation is equivalent to abnormally low levels of carbon dioxide in the blood (e.g. hypocapnia, hypocapnea, or hypocarbia) caused by overbreathing. Hyperventilation is the opposite of hypoventilation (e.g. underventilation) that often occurs in patients with lung disease and results in high levels of carbon dioxide in the blood (e.g. hypercapnia or hypercarbia).

A low partial pressure of carbon dioxide in the blood causes alkalosis, because CO2 is acidic in solution and reduced CO2 makes blood pH more basic, leading to lowered plasma calcium ions and nerve and muscle excitability. This condition is undesirable in cardiac patients since it can increase probability of cardiac arrhythmias.

Alkalemia may be defined as abnormal alkalinity, or increased pH of the blood. Respiratory alkalosis is a state due to excess loss of carbon dioxide from the body, usually as a result of hyperventilation. Compensated alkalosis is a form in which compensatory mechanisms have returned the pH toward normal. For example, compensation can be achieved by increased excretion of bicarbonate by the kidneys.

Compensated alkalosis at rest can become uncompensated during exercise or as a result of other changes of metabolic balance. Thus the invented method is applicable to treatment of both uncompensated and compensated respiratory alkalosis.

Tachypnea means rapid breathing. For the purpose of this disclosure a breathing rate of about 6 to 16 breaths per minute at rest is considered normal but there is a known benefit to lower rate of breathing in cardiac patients. Reduction of tachypnea resulting from the removal of afferent nerve input to the respiratory center in the brain from chemoreceptors in carotid bodies can be expected to reduce respiratory dead space, increase breathing efficiency, and increase parasympathetic tone.

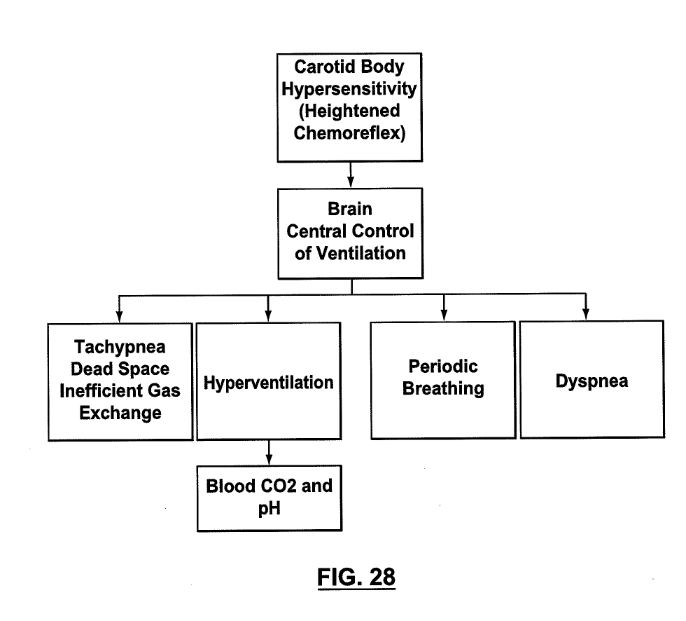

Carotid body hyperactivity or hypersensitivity may be involved in a disease progression responsible for debilitating symptoms independently or in addition of its ability to heighten central sympathetic tone. For example, the carotid body may exert direct influence on ventilation through its primary function as blood gas concentration sensing organ. If a carotid body becomes hypersensitive in the process of disease progression it may become responsible for abnormal physiologic conditions such as: unnecessarily rapid breathing (tachypnea), periodic breathing, hyperventilation, apnea, excessive blood alkalinity, limited ability to exercise and sensation of dyspnea. Carotid body hyperactivity or hypersensitivity can result in rapid breathing at rest, during sleep and in response to exercise that, among other effects, can result in increased dead space, ventilation-perfusion mismatch and inefficient utilization of lungs for gas exchange. A physiological connection of heightened chemoreflex with ventilatory effects is shown in FIG. 28. In historic surgeries described as glomectomy CB removal was demonstrated to reduce dyspnea (sensation of breathlessness) in patients with pulmonary disease (i.e. asthma and COPD). In patients with heart failure dyspnea often limits their ability to exercise. CB ablation/excision may result in reduction of the perception of dyspnea, and improvement in exercise tolerance. One possible mechanism is through neurogenically altering the contribution of the carotid body to establishing the set point and gain of the central chemoreceptor located in the brain, which may be shifted due to the increased CSNA. Thus reduction of afferent signals from carotid body may result in the reduction of sympathetic drive that alter the perception of dyspnea in heart failure that is somewhat analogous to the mechanism of panic disorder. In addition carotid body ablation may result in the reduced tachypnea and hyperventilatory response to exercise in heart failure patients that will result in more efficient ventilation and better perfusion-ventilation match in the lung. All these factors should contribute to the ability of heart failure patients to exercise longer without sensation of breathlessness. Benefits of exercise in heart failure are well known and even modest improvement in exercise duration can extend life of these patients.

Inventors observed increased ability to exercise (measured during metabolic stress test and six minute walk test in patients with CHF that underwent both unilateral and bilateral surgical ablation of carotid body. In general these patients exercised longer one month after surgery than they did before. The surgeries and exercise testing was performed by investigators in Wroclaw, Poland at the request of Inventors.

Normally, food is absorbed into the bloodstream in the form of sugars, such as glucose, and other basic substances. An increase in glucose in the bloodstream signals the pancreas to increase the secretion of hormone insulin. Insulin attaches to cells, removing sugar from the bloodstream so that it can be used for energy.

In insulin resistance, the body's cells have a diminished ability to respond to the action of the insulin. To compensate for the insulin resistance, the pancreas secretes more insulin. People with this syndrome have insulin resistance and high levels of insulin in the blood as a marker of the disease rather than a cause. Over time people with insulin resistance can develop diabetes as the high insulin levels can no longer compensate for elevated levels of glucose. In general so called metabolic syndrome or Insulin Resistance (IR) syndrome is a name for a group of risk factors that occur together and increase the risk for coronary artery disease, stroke, and type II diabetes. Metabolic syndrome is becoming more and more common in the United States. Researchers are not sure whether the syndrome is due to one single cause, but all of the risks for the syndrome are related to obesity.

In other words insulin resistance (IR) is a physiological condition where the natural hormone insulin becomes less effective at lowering blood sugars. The resulting increase in blood glucose may raise blood glucose levels outside the normal range and cause adverse health effects, depending on dietary conditions. Certain cell types such as fat and muscle cells require insulin to absorb glucose. When these cells fail to respond adequately to circulating insulin, blood glucose levels rise. The liver helps regulate glucose levels by reducing its secretion of glucose in the presence of insulin. This normal reduction in the liver's glucose production may not occur in people with insulin resistance. Insulin resistance in muscle and fat cells reduces glucose uptake (and also local storage of glucose as glycogen and triglycerides, respectively), whereas insulin resistance in liver cells results in reduced glycogen synthesis and storage and a failure to suppress glucose production and release into the blood.

We suggest that elevated CSNA contributes to insulin resistance in two ways: 1) increases of sympathetic activity redistributes blood flow from insulin sensitive tissue, such as peripheral muscle, to insulin insensitive tissue, such as visceral organs, resulting in an increase in resistance to the action of insulin. This action will result in development of abnormalities in glucose deposition, particularly after meals; and 2) increases of sympathetic activity cause increases in hepatogenic glucose production, which raises glucose, often associated with increases in fasting glucose levels. These two actions of pathologically increased sympathetic nerve activity result in increases of glucose levels after eating a meal and increases in fasting glucose, with neurogenically mediated insulin resistance and consequent elevations of insulin levels.

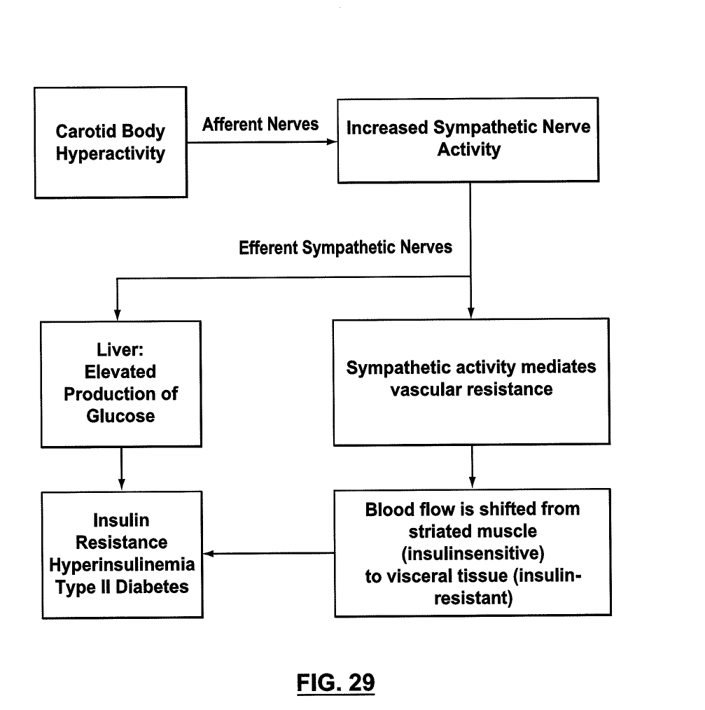

We propose that chronic increases in carotid body activity (tonic) or increases in chemosensitivity might increase central sympathetic tone causing an increase of gluconeogenesis (formation of glucose from glycogen stores in the liver) or an increase in resistance to the action of insulin as illustrated in FIG. 29. Sympathetic hyperactivation causes a change in vascular resistance and induces insulin resistance. Insulin resistance contributes to the “metabolic syndrome,” which is associated with increased cardiovascular morbidity and mortality. It is known that inhibition of the Central Sympathetic Nerve Activity (for example with sympatholytic drugs) reduces insulin resistance and improves glucose metabolism.

What is uniquely proposed is that the carotid body, either through its tonic contribution to CSNA, or intermittent contribution to sympathetic drive via chemo hypersensitivity, can underlie the sympathetic activation that causes insulin resistance, elevated insulin levels, and the risk for developing type II diabetes mellitus (T2DM). This constellation of findings, metabolic syndrome, elevated insulin and elevated glucose levels in the presence of excess sympathetic drive leads to the novel therapy.

We suggest that reduction of CSNA following carotid body ablation will simultaneously reduce gluconeogenesis and insulin resistance. Specifically we expect CBA to result in redistribution of blood from splanchnic circulation to skeletal muscle where glucose can be efficiently metabolized.

Essential hypertension and insulin resistance may be different phenotypical manifestations of excess sympathetic drive. Reduction of sympathetic drive may, therefore, reduce essential hypertension and insulin resistance in parallel. These two conditions are frequently encountered in the same patient. Carotid body ablation could reduce insulin resistance and therefore reduce insulin levels. Carotid body ablation could also reduce glucose levels, and facilitate glucose control in established diabetics.

Carotid body ablation may reduce the incidence of type II diabetes in patients with excessive sympathetic drive. Reduction of the incidence of diabetes will be seen in sympathetically mediated diseases such as hypertension, heart failure, chronic renal disease, polycystic ovary syndrome as well as other diseases common in the elevation of sympathetic drive. Carotid body ablation may prevent development of diabetes or reverse the condition of type II diabetes in select newly diagnosed diabetics.

FIG. 40 illustrates reduction of Homeostatic Measurement Assessment-Insulin Resistance (HOMA-IR), commonly used for detection of Glucose Intolerance, in one patient that had unilateral carotid body removal. Six months after surgery patient's insulin resistance e is significantly reduced. Patient was studies upon the Inventors request as part of the CHF study in Wroclaw, Poland.

Sodium retention (sodium avidity) by the kidney is not unique to heart failure but is particularly important in the context of heart failure progression. Heart failure patients often develop resistance to diuretic medication, retain sodium and fluid, even when optimally treated. They are frequently admitted to emergency rooms and hospitals as a result of pulmonary edema and fluid overload.

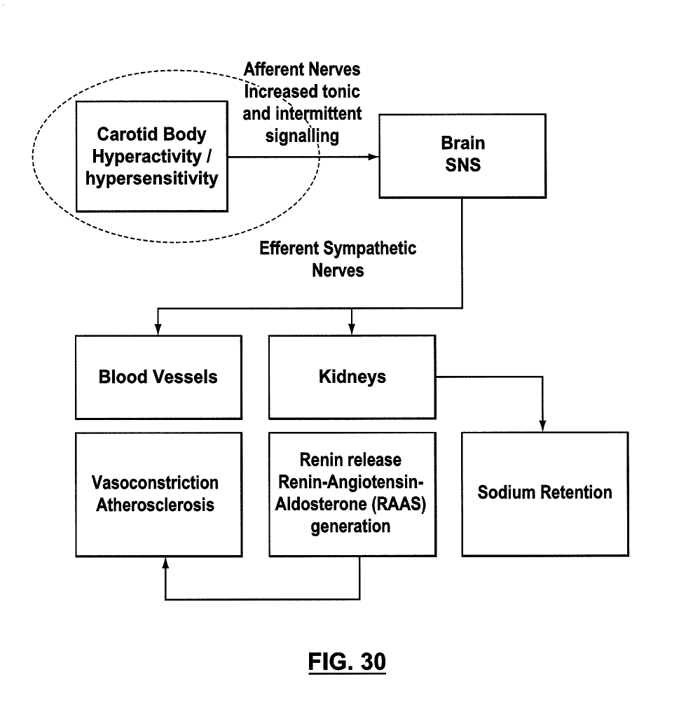

It is known that efferent sympathetic nerves play an important role in the mechanism of sodium retention by kidneys. In patients with heart failure renal sympathetic nerve activity (RSNA) is greatly increased. Interruption of renal nerves and reduction of RSNA has proven benefit. RSNA is mediated by CSNA that is elevated in heart failure likely more than in other sympathetically mediated disease. Carotid body hyperactivity and hypersensitivity results in the increased efferent CSNA. A connection between carotid body hyperactivity and sodium retention through the CNS is shown in FIG. 30. Carotid body ablation has been conceived as a treatment for sodium retention with the intention of reducing afferent neural signaling from the carotid body to the CNS resulting in a reduction of CSNA followed by a reduction of efferent sympathetic nerve signals to the kidney, reduction of RSNA (efferent and afferent) and a resulting reduction of sodium retention and reduction of secretions of renal hormones (renin). Reduction of renin secretion has additional benefit of the deactivation of the renin-angiotensin-aldosterone system (RAAS) is a hormone system that regulates blood pressure and water (fluid) balance.

Efferent sympathetic nerves to the kidneys terminate in the blood vessels, the juxtaglomerular apparatus, and the renal tubules. Increase in RSNA causes increased renin release, increased sodium reabsorption (retention), and a reduction of renal blood flow.

These components of the neural regulation of renal function are stimulated considerably in disease states characterized by heightened sympathetic tone and clearly contribute to the elevations of blood pressure and abnormal sodium retention. The reduction of renal blood flow and glomerular filtration rate as a result of renal sympathetic efferent stimulation is likely a major contributor to the loss of renal function in cardiorenal syndrome, a severe complication of chronic heart failure.

The negative predictive value of renal sympathetic activation with all-cause mortality and risk of heart transplantation in patients with congestive heart failure, independent of overall sympathetic activity, glomerular filtration rate, and left ventricular ejection fraction underscores importance of reduction of RSNA in the treatment of heart failure. These findings clearly suggest that treatment regimens that further reduce RSNA have the potential to improve survival in patients with heart failure.

Under request of the inventors based on concepts disclosed herein, experiments in rabbits with induced heart failure and in rats with hypertension convincingly demonstrated that carotid body ablation may be an effective treatment for reducing RSNA.

Both Chronic Renal Disease (CRD) and End Stage Renal Disease (ESRD) are characterized by heightened sympathetic nervous activation. In patients with chronic kidney disease, progression of renal failure can be delayed by the centrally acting sympatholytic agent moxonidine. Moxonidine has also been demonstrated to reduce microalbuminuria in normotensive patients with type I diabetes mellitus, in the absence of any significant blood pressure changes. In patients with ESRD, plasma levels of norepinephrine above the median have been demonstrated to be predictive for both all-cause death and death from cardiovascular disease. Renal sympathetic efferent signals also contribute to renal inflammation. Reduction of renal sympathetic efferent signaling is expected to reduce renal inflammation, and favorably impact specific renal disorders such as glomerulronephritis, post ischemic renal inflammation and diabetic renal disorders. Carotid body ablation may be instrumental in treatment of CRD through the mechanism of reduction of CSNA and RSNA.

A method has been conceived for reducing renal sympathetic efferent signaling via carotid body ablation to protect against advancing CRD. There is a growing literature suggesting that renal sympathetic denervation (RDN) may prove protective against advancing renal disease of several etiologies. Carotid body ablation may be renal protective via the indirect reduction of renal sympathetic efferent nerve traffic. Differently from direct renal nerve ablation (various technologies exist for RDN), carotid body ablation reduces renal signaling consequent to the selective removal of excessive tonic or chemosensitive signals emanating from the carotid body, while preserving the kidney's ability to respond to efferent signals derivative from other sympathetic sources.

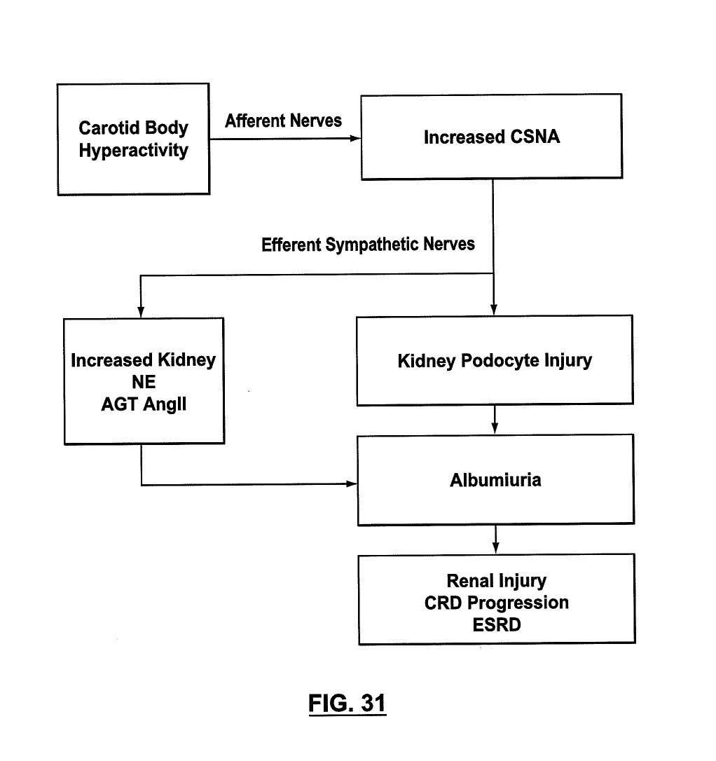

A connection between carotid body hyperactivity and renal injury, CRD progression, and ESRD is illustrated in FIG. 31. Afferent signals from the carotid body lead to increased CSNA that adversely affects the progression of renal disease. This counterintuitive logic suggests that in conditions where excessive CSNA and RSNA underlie advancing renal injury, ablation of carotid body, known for control of respiration, may be protective of kidneys. Reno-protective effect of carotid body ablation is expected through a reduction of CSNA and RSNA and a reduction of the activity of the RAAS, reduction of renal norepinephrine (NE), angiotensin (AGT) and angiotensin II (AngII).

CRD of many etiologies and particularly diabetic nephropathy in humans is characterized by increased urinary albumin excretion (albuminuria), which often progresses to proteinuria, one of the most important prognostic risk factors for kidney disease progression. Glomerular visceral epithelial cells, or podocytes, play a critical role in maintaining the structure and function of the glomerular filtration barrier. Injury and reduction of density of podocytes may be one mechanism through which increased RSNA contributes to the progression of CRD.

Acute decompensated heart failure (ADHF) is a worsening of the symptoms, typically shortness of breath (dyspnea), edema and fatigue, in a patient with existing heart failure. It is a frequent and serious complication that requires hospitalization and can be life threatening. In ADHF back up of blood in vessels and the lungs causes buildup of fluid (congestion) in the tissues.

Chronic stable heart failure may easily decompensate. This most commonly results from pneumonia, myocardial infarction, arrhythmias, uncontrolled hypertension, or a patient's failure to maintain a fluid restriction, diet or medication. Other well recognized precipitating factors include anemia and hyperthyroidism which place additional strain on the heart muscle. Excessive fluid or salt intake, and medication that cause fluid retention may also precipitate decompensation.

It has recently been recognized that the majority of patients with heart failure experience acute decompensations (congestion) without increases of total body salt and water (weight gain). Further, increases in intravascular pressure, occur in the absence of weight changes, implying that volume shifts not volume gains are the underlying feature. Human mesenteric vessels are the reservoirs of volume, and are extensively sympathetically innervated.

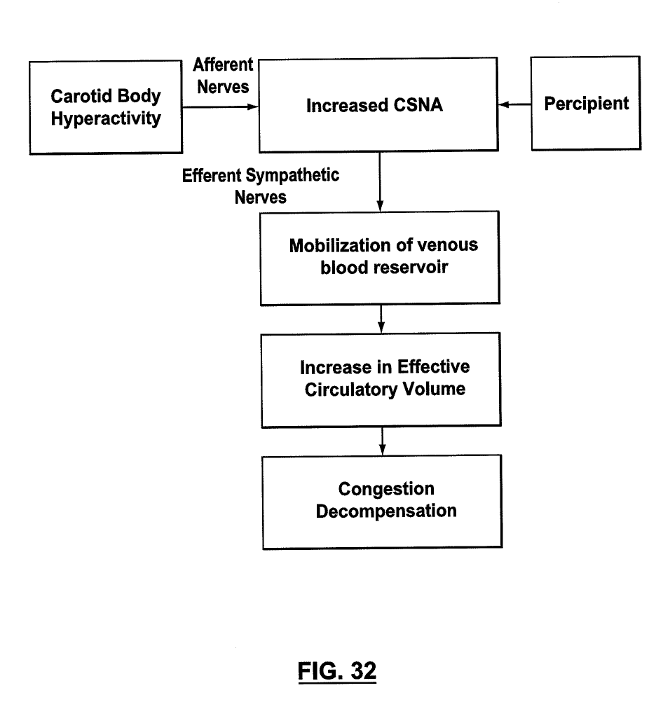

FIG. 32 illustrates a connection between carotid body hyperactivity and decompensation (congestion) via afferent signals increasing CSNA. Therefore, a method of preventing or relieving sympathetically mediated acute decompensation in patients with congestive heart failure via carotid body ablation has been conceived. Carotid body ablation may reduce the sympathetic signals that cause congestion to occur due to changes in regional capacitance.

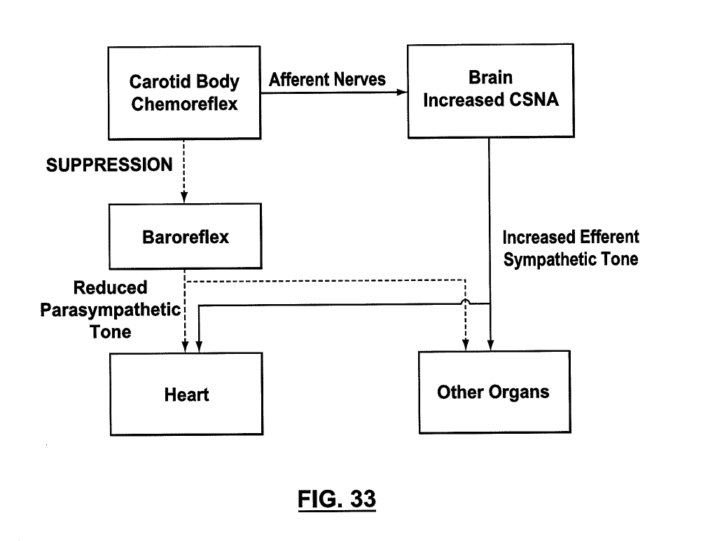

The importance of parasympathetic tone in cardiovascular disease is well established. Particularly in hypertension and heart failure there is increasing evidence of frequently suppressed baroreflex. Increased baroreflex and vagal (parasympathetic) tone have proven benefit in those conditions as demonstrated by electro stimulation of vagus and baroreceptors. The carotid body contributes directly to autonomic imbalance by increasing central sympathetic tone and indirectly via reducing the baroreflex and baroreflex sensitivity. Thus, in conditions characterized by sympathetic nervous system hyperactivity and high chemosensitivity, carotid body ablation will reduce efferent systemic and renal sympathetic nervous activation and may indirectly increase baroreflex and vagal tone as illustrated in FIG. 33.

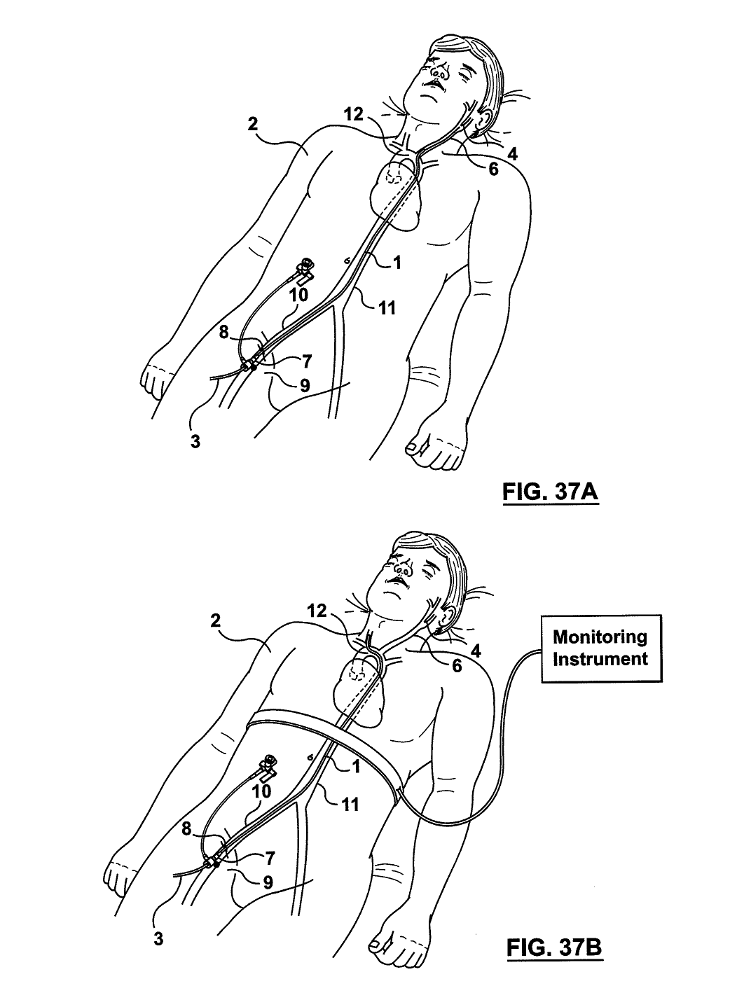

FIGS. 37A and 37B illustrate individual diagnostic testing of chemosensitivity by injection of substance into left and right common carotid arteries.

FIG. 37A is a schematic view showing endovascular access with a catheter to a left common carotid artery of a patient lying in supine position. The catheter may be a diagnostic endovascular catheter inserted through an introducer sheath in a femoral artery at the groin area of the patient. The catheter may be advanced into the left common carotid artery over a wire. The same sheath, wire and rout may be used to insert an ablation catheter and advance it to the carotid bifurcation to ablate the carotid body chemoreceptors. The catheter may be positioned just at or just past the ostium of the left common carotid. It is understood that there are variations in human anatomy and this figure is a schematic. Importantly, if a substance is injected from the distal tip of the catheter in to a left common carotid artery it will selectively affect the left carotid body on the first pass and only reach right carotid body after completing full cycle of circulation and highly diluted. Thus the immediate (starting within seconds and ending in few minutes) respiratory and hemodynamic response to the injection can be selectively contributed to the left CB.

FIG. 37A is a schematic view showing the same endovascular access with a catheter repositioned from the left common carotid to the right common carotid artery of a patient. Injection of a substance that stimulates a carotid body and peripheral chemoreflex may be now made selectively to stimulate the right carotid body.

The patient is shown wearing a respiratory belt suitable to measure breath to breath respiration. Other physiologic sensors such as air flow meters, ECG or pulse monitors can be added to the patient monitoring device. A patient monitoring device may be equipped with software and a user interface in order to inform the operator of the magnitude of the patient's response to the injection of the substance that stimulates peripheral chemoreflex (e.g. adenosine).

A substance chosen as an example of an injectable drug that can be used for selective stimulation of carotid bodies is adenosine. It is known that adenosine causes increased carotid body activity and augmented ventilation. It impacts primarily the carotid bodies and is not expected to cross the blood-brain barrier. Its half-life in circulation is only on the order of 10 seconds and delayed affects are not expected. Thus physiologic effects of adenosine injection into a carotid artery can be expected to be short, immediate and directly attributable to one of two carotid bodies, depending on the site of injection. In addition, if adenosine or similar substance is injected into arterial circulation elsewhere (such as into the femoral sheath or aorta) the effect can be assumed to be a sum of effects on both carotid bodies.

Adenosine injection directly into arterial circulation is currently used in Fractional Flow Reserve (FFR) measurement in cardiac catheterization laboratories. It is understood that there are other chemical substances that can be used to stimulate carotid bodies and peripheral chemoreflex.

Potential use of the described method is in giving physicians instant feedback regarding success or failure of a carotid body modulation procedure during the procedure and to help select a more active carotid body for a unilateral procedure.

As discussed previously, adenosine activates carotid bodies. An intra-carotid injection of adenosine would increase respiration rate, minute ventilation and decrease end-tidal CO₂. These parameters may be measured with relatively simple instruments known in the field of cardiology and pulmonology.

An increase in any of these parameters after application of ablation energy compared to their baseline values could imply unsuccessful carotid body modulation. Another round of energy application may be necessitated. It is expected that successful ablation of a carotid body will result in substantial reduction (e.g. >50% and possibly >90%) of respiratory response to adenosine compared to baseline before ablation.

On the other hand, a lack of reduction in response could imply unsuccessful carotid body modulation, wherein a physician may decide to repeat a procedure and catheter energy delivery elements can be repositioned or energy delivered (power or time) can be increased as a result of this information. In addition or alternatively, a different energy delivery modality can be tried such as pulsed RF. If single-sided ablation is ultimately not successful an operator may reposition the ablation catheter and attempt therapy on the other side instead.

It may be possible to improve or further augment the response to adenosine by making the patient mildly hypoxic before injection by use of a rebreathing mask.

Injection of low dose adenosine into a carotid artery proximal of a carotid bifurcation to activate a carotid body is proposed as a peri-procedural measure of technical success. A bolus of adenosine may result in acute increase ventilation, HR, BP SVR, and sympathetic nervous system changes within 10-30 seconds. The response may be dose dependent and some reasonable dose range study will be required to find an optimal dose. The bolus must be administered close to the CB to work (at a common carotid ostium or at a carotid bifurcation). Both an internal lumen in the energy delivery catheter or the sheath may be used for injections. Alternatively, a separate infusion catheter may be used. An adenosine test can be performed before and after a carotid body modulation procedure to confirm ablation of a carotid body and reduction of chemoreflex. If a carotid body modulation procedure is technically unsuccessful a physician may choose, or an algorithm may suggest, to re-ablate at the same location, move location, reposition energy delivery elements, for example improve apposition of electrodes, or change an energy setting (e.g. apply more energy, apply energy over longer time or at different delivery algorithm such as pulsed RF). For example, in the case of bipolar RF ablation of a carotid septum, power setting of a RF generator may be increased from 6 Watt to 8 Watt or 10 Watt and time of energy delivery can be increased from 30 sec to 45 sec or 60 sec. CB removal or modulation (removing CB afferent input) results in the elimination of the immediate ventilatory response to adenosine injection. There may be also some heart rate (RR) or blood pressure (BP) changes due to carotid body activation and SNS activation from adenosine injection as the injection and observation window are very short such as 5, 10 seconds and no longer than 60 seconds.

What is claimed is:

1. A method of planning for a procedure that reduces afferent nerve activity of a carotid body, comprising
providing at least one image of a subject's carotid vasculature region;
determining a characteristic of at least one of a vagus nerve, hypoglossal nerve, and sympathetic nerve; and
making a determination about the afferent nerve activity reducing procedure based on the characteristic of at least one of the vagus nerve, hypoglossal nerve, and sympathetic nerve.

2. The method of claim 1 wherein determining a characteristic of at least one of a vagus nerve, hypoglossal nerve, and sympathetic nerve comprises determining a position of a carotid body relative to at least one of a vagus nerve, hypoglossal nerve, and sympathetic nerve, and making a determination about the afferent nerve activity reducing procedure comprises making a determination about the afferent nerve activity reducing procedure based on the position of the carotid body relative to at least one of the vagus nerve, hypoglossal nerve, and sympathetic nerve.

3. The method of claim 1 wherein determining a characteristic of at least one of a vagus nerve, hypoglossal nerve, and sympathetic nerve comprises delivering a stimulator in the area of the carotid vasculature and monitoring for a response indicative of stimulation of at least one of the vagus nerve, hypoglossal nerve, and sympathetic nerve.

4. The method of claim 3 wherein delivering a stimulator comprises delivering at least one of thermal and electrical energy in the area of the carotid vasculature.

5. The method of claim 3 wherein delivering a stimulator comprises delivering a chemical agent in the area of the carotid vasculature.

6. The method of claim 3 wherein monitoring for a response indicative of stimulation of at least one of the vagus nerve, hypoglossal nerve, and sympathetic nerve comprises monitoring for a motor response.

7. The method of claim 3 wherein monitoring for a response indicative of stimulation of at least one of the vagus nerve, hypoglossal nerve, and sympathetic nerve comprising monitoring for at least one of a cardiovascular response and a respiratory response.

8. A method of planning for a procedure that reduces afferent nerve activity of a carotid body, comprising
providing at least one image of a subject's carotid vasculature;
determining if an atherosclerosis is present in the carotid vasculature; and
making a determination about the afferent nerve activity reducing procedure based on the presence or absence of an atherosclerosis in the at least one image.

9. The method of claim 8 wherein the investigating step investigates an image that shows a common carotid artery, and the determining step determines if an atherosclerosis is present in the common carotid artery.

10. The method of claim 8 wherein the investigating step investigates an image that shows at least a portion of an aortic arch, and the determining step determines if an atherosclerosis is present in the aortic arch.

11. The method of claim 8 wherein the investigating step investigates an image that shows an external carotid artery, and the determining step determines if an atherosclerosis is present in the external carotid artery.

12. The method of claim 8 wherein making a determination about the afferent nerve activity reducing procedure comprises making a determination about whether or not to perform the procedure.

13. The method of claim 8 wherein making a determination about the afferent nerve activity reducing procedure comprises making a determination about a vascular approach to a treatment site for the procedure.

14. A method of planning for a procedure that reduces afferent nerve activity of a carotid body, comprising
providing at least one image of a subject's carotid vasculature region;
assessing a distance from a carotid body to at least one anatomical structure using the at least one image; and
making a determination about the afferent nerve activity reducing procedure based on the assessed distance from the carotid body to the at least one anatomical structure.

15. The method of claim 14 wherein assessing a distance from a carotid body to at least one anatomical structure using the at least one image comprises assessing a distance from the carotid body to an internal carotid artery.

16. The method of claim 14 wherein assessing a distance from a carotid body to at least one anatomical structure using the at least one image comprises assessing a distance from the carotid body to an external carotid artery.

17. The method of claim 14 wherein assessing a distance from a carotid body to at least one anatomical structure using the at least one image comprises assessing a distance from the carotid body to a carotid artery bifurcation.

18. The method of claim 14 wherein making a determination about the afferent nerve activity reducing procedure based on the assessed distance from the carotid body to the at least one anatomical structure comprises deciding where to position a treatment device within the vasculature to perform the procedure.

19. The method of claim 14 wherein the assessing step is performed automatically by an algorithm.

20. The method of claim 14 wherein the assessing step is performed manually.

21. A method of planning for a procedure that reduces afferent nerve activity of a carotid body, comprising
determining that an ablation procedure on one of a right carotid body and a left carotid body will better treat a disease associated with heightened carotid body activation than an ablation procedure on the other of the right carotid body and a left carotid body; and
performing the procedure on the right or the left carotid body based on the determining step.

22. The method of claim 21 further comprising assessing whether a disease associated with heightened carotid body activation has been satisfactorily treated.

23. The method of claim 22 further comprising performing the procedure on the other of the left and right carotid body if it is determined that the disease has not been satisfactorily treated.

24. The method of claim 21 wherein determining that an ablation procedure on one of a right carotid body and a left carotid body will better treat a disease associated with heightened carotid body activation comprises testing chemosensitivity of at least one of a left carotid body and a right carotid body.

25. The method of claim 21 wherein determining that an ablation procedure on one of a right carotid body and a left carotid body will better treat a disease associated with heightened carotid body activation comprises assessing the size of the right carotid body.

26. The method of claim 21 wherein determining that an ablation procedure on one of a right carotid body and a left carotid body will better treat a disease associated with heightened carotid body activation comprises selectively stimulating at least one of the left and right carotid bodies, and measuring a response to the selective stimulation of the at least one left and right carotid bodies.

27. The method of claim 26 wherein selectively stimulating comprises exposing the left or right carotid body to a stimulant.

28. A method of planning for a procedure that reduces afferent nerve activity of a carotid body, comprising
providing at least one image of a subject's carotid vasculature region;
determining if a carotid body is located at least partially within a carotid septum using the at least one image; and
making a determination about the afferent nerve activity reducing procedure based on the determination if the carotid body is located at least partially within the carotid septum.

29. The method of claim 28 wherein making a determination about the afferent nerve activity reducing procedure comprises making a determination about whether to perform the procedure or not based on whether the carotid body is located at least partially within a carotid septum or not.

30. The method of claim 28 wherein the making a determination step comprises making a determination not to perform the procedure if the carotid body is not substantially located within the carotid septum.

31. The method of claim 28 wherein making a determination about the afferent nerve activity reducing procedure comprises making a determination to at least partially ablate the carotid septum.

32. A method of planning for a procedure that reduces afferent nerve activity of a carotid body, comprising
providing at least one image of a subject's carotid vasculature; and
making a determination about an aspect of energy delivery for the afferent nerve activity reducing procedure based on the image of the subject's carotid vasculature.

33. The method of claim 32 making a determination about an aspect of energy delivery comprises selecting one of a plurality of different energy modalities for the procedure based on the at least one image.

34. The method of claim 32 wherein making a determination comprises selecting RF energy as the energy modality for the procedure.

35. The method of claim 32 making a determination about an aspect of energy delivery comprises selecting one of a plurality of different energy parameters for the procedure based on the at least one image.

36. The method of claim 35 wherein making a determination about an aspect of energy delivery comprises selecting a power at which energy is delivered for the procedure.

37. The method of claim 35 wherein making a determination about an aspect of energy delivery comprises selecting a duration during which energy is delivered for the procedure.

38. A method of planning for a procedure that reduces afferent nerve activity of a carotid body, comprising
providing at least one image of a subject's carotid vasculature; and
making a determination about a vascular approach for a treatment device for the afferent nerve activity reducing procedure based on the at least one image of the subject's carotid vasculature.

39. The method of claim 38 wherein making a determination about a vascular approach comprises determining an access point for a treatment device for the afferent nerve activity reducing procedure.

40. The method of claim 38 wherein making a determination about a vascular approach comprises determining a navigation route to a treatment site for the treatment device.

41. The method of claim 38 wherein making a determination about a vascular approach for a treatment device for the afferent nerve activity reducing procedure based on the image of the subject's carotid vasculature comprises recognizing the presence or absence of an atherosclerosis in the subject's vasculature.

42. The method of claim 38 wherein making a determination about a vascular approach for a treatment device comprises selecting one or a plurality of treatment devices based on the at least one image of the subject's carotid vasculature.

43. The method of claim 38 wherein making a determination about a vascular approach for a treatment device comprises determining whether or not to access an external carotid artery.

44. The method of claim 38 wherein making a determination about a vascular approach for a treatment device is performed by an algorithm.

45. The method of claim 38 wherein making a determination about a vascular approach for a treatment device comprises manually making a determination about a vascular approach for a treatment device.

46. A method of performing a procedure on a subject that reduces afferent nerve activity of a carotid body, comprising
providing an image of the subject's carotid vasculature;
measuring a distance of about 15 mm from a carotid artery bifurcation along a lumen of external carotid artery to estimate a first position range;
positioning an energy delivery device in an external carotid artery in the first range; and
activating the energy delivery device to ablate tissue within the carotid septum.

47. The method of claim 46 wherein activating the energy delivery device to ablate tissue within the carotid septum comprises ablating at least a part of a carotid body.
